SEQUENCE LISTING (324 sequences)

SEQ ID NO: 1 - DNA, 20 bp, Homo sapiens
  source 1..20: mol_type = genomic DNA
cgggctggcaaccgattccg

SEQ ID NO: 2 - DNA, 20 bp, Homo sapiens
  source 1..20: mol_type = genomic DNA
ccgattccggggtcccccaa

SEQ ID NO: 3 - DNA, 20 bp, Homo sapiens
  source 1..20: mol_type = genomic DNA
cggaatcccgaataaccaaa

SEQ ID NO: 4 - DNA, 20 bp, Homo sapiens
  source 1..20: mol_type = genomic DNA
cccggaatcggttgccagcc

SEQ ID NO: 5 - DNA, 20 bp, Homo sapiens
  source 1..20: mol_type = genomic DNA
gttattcgggattccgtgtc

SEQ ID NO: 6 - DNA, 20 bp, Homo sapiens
  source 1..20: mol_type = genomic DNA
gacacggaatcccgaataac

SEQ ID NO: 7 - DNA, 20 bp, Homo sapiens
  source 1..20: mol_type = genomic DNA
ccagcgcctgtaagatgctc

SEQ ID NO: 8 - DNA, 20 bp, Homo sapiens
  source 1..20: mol_type = genomic DNA
ccagagcatcttacaggcgc

SEQ ID NO: 9 - DNA, 20 bp, Homo sapiens
  source 1..20: mol_type = genomic DNA
ccttacctggatggtgttat

SEQ ID NO: 10 - DNA, 20 bp, Homo sapiens
  source 1..20: mol_type = genomic DNA
ccaataacaccatccaggta

SEQ ID NO: 11 - DNA, 20 bp, Homo sapiens
  source 1..20: mol_type = genomic DNA
cttggccaataacaccatcc

SEQ ID NO: 12 - DNA, 20 bp, Homo sapiens
  source 1..20: mol_type = genomic DNA
cagagactacaagccaggct

SEQ ID NO: 13 - DNA, 20 bp, Homo sapiens
  source 1..20: mol_type = genomic DNA
ctgggcagagactacaagcc

SEQ ID NO: 14 - DNA, 20 bp, Homo sapiens
  source 1..20: mol_type = genomic DNA
tacacgtgccagtgcctgcc

SEQ ID NO: 15 - DNA, 20 bp, Homo sapiens
  source 1..20: mol_type = genomic DNA
tgtaccgtcacaagtctcca

SEQ ID NO: 16 - DNA, 20 bp, Homo sapiens
  source 1..20: mol_type = genomic DNA
taccgtcacaagtctccatg

SEQ ID NO: 17 - DNA, 20 bp, Homo sapiens
  source 1..20: mol_type = genomic DNA
cgcgtctttctcgtctttct

SEQ ID NO: 18 - DNA, 20 bp, Homo sapiens
  source 1..20: mol_type = genomic DNA
cggcagctcagctggaacca

SEQ ID NO: 19 - DNA, 81 bp, Homo sapiens
  source 1..81: mol_type = genomic DNA
gacgctttcccgattcttcgacaaagtccaggacctgggcagagactacaagccaggcttggccaataacaccatccaggt

SEQ ID NO: 20 - DNA, 81 bp, Homo sapiens
  source 1..81: mol_type = genomic DNA
acctggatggtgttattggccaagcctggcttgtagtctctgcccaggtcctggactttgtcgaagaatcgggaaagcgtc

SEQ ID NO: 21 - protein, 26 aa, Homo sapiens
TLSRFFDKVQDLGRDYKPGLANNTIQ

SEQ ID NO: 22 - DNA, 250 bp, Homo sapiens
  source 1..250: mol_type = genomic DNA
cttcatgcgggccctctggcccatggctcactaggtctgtgtccacatccctccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacaggtaggtccctgggtct

SEQ ID NO: 23 - DNA, 250 bp, Homo sapiens
  source 1..250: mol_type = genomic DNA
agacccagggacctacctgtgcctgcaggataactgaagttcaacagcccattggaaaggttcttgctcaggcctctgaggacatcctctaggccatccagcaggtgactggccacacagtgctgctgtaagcggggcagggtctccaggtccccaggggcctccagcagctcatccagcgcctgtaagatgctctggagggatgtggacacagacctagtgagccatgggccagagggcccgcatgaag

SEQ ID NO: 24 - protein, 59 aa, Homo sapiens
SILQALDELLEAPGDLETLPRLQQHCVASHLLDGLEDVLRGLSKNLSNGLLNFSYPAGT

SEQ ID NO: 25 - DNA, 79 bp, synthetic construct
  source 1..79: mol_type = other DNA
gacgctttcccgattcttcgacaaagtccaggacctgggcagagactacaagccaggcttggccaataactccaggtaa

SEQ ID NO: 26 - DNA, 79 bp, synthetic construct
  source 1..79: mol_type = other DNA
ttacctggagttattggccaagcctggcttgtagtctctgcccaggtcctggactttgtcgaagaatcgggaaagcgtc

SEQ ID NO: 27 - protein, 25 aa, synthetic construct
TLSRFFDKVQDLGRDYKPGLANNSR

SEQ ID NO: 28 - DNA, 250 bp, synthetic construct
  source 1..250: mol_type = other DNA
atgcgggccctctggcccatggctcactaggtctgtgtccacatccctccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacaggtaggtccctgggtctgccc

SEQ ID NO: 29 - DNA, 250 bp, synthetic construct
  source 1..250: mol_type = other DNA
gggcagacccagggacctacctgtgcctgcaggataactgaagttcaacagcccattggaaaggttcttgctcaggcctctgaggacatcctctaggccatccagcaggtgactggccacacagtgctgctgtaagcggggcagggtctccaggtccccaggggcctccagcagctcatccagcgcctgtaagatgctctggagggatgtggacacagacctagtgagccatgggccagagggcccgcat

SEQ ID NO: 30 - DNA, 20 bp, Homo sapiens
  source 1..20: mol_type = genomic DNA
cggaatcggttgccagcccg

SEQ ID NO: 31 - DNA, 20 bp, Homo sapiens
  source 1..20: mol_type = genomic DNA
ttgggggaccccggaatcgg

SEQ ID NO: 32 - DNA, 20 bp, Homo sapiens
  source 1..20: mol_type = genomic DNA
tttggttattcgggattccg

SEQ ID NO: 33 - DNA, 20 bp, Homo sapiens
  source 1..20: mol_type = genomic DNA
ggctggcaaccgattccggg

SEQ ID NO: 34 - protein, 56 aa, synthetic construct
ASYRRWMSCWRPLGTWRPCPAYSSTVWPVTCWMARMSSEAARTFPMGCTSVILQAQ

SEQ ID NO: 35 - DNA, 80 bp, Homo sapiens
  source 1..80: mol_type = genomic DNA
gacgctttcccgattcttcgacaaagtccaggacctgggcagagactacaagccaggcttggccaataacaccatccagg

SEQ ID NO: 36 - DNA, 20 bp, Homo sapiens
  source 1..20: mol_type = genomic DNA
gagcatcttacaggcgctgg

SEQ ID NO: 37 - DNA, 20 bp, Homo sapiens
  source 1..20: mol_type = genomic DNA
gcgcctgtaagatgctctgg

SEQ ID NO: 38 - DNA, 20 bp, Homo sapiens
  source 1..20: mol_type = genomic DNA
ataacaccatccaggtaagg

SEQ ID NO: 39 - DNA, 20 bp, Homo sapiens
  source 1..20: mol_type = genomic DNA
tacctggatggtgttattgg

SEQ ID NO: 40 - DNA, 20 bp, Homo sapiens
  source 1..20: mol_type = genomic DNA
ggatggtgttattggccaag

SEQ ID NO: 41 - DNA, 20 bp, Homo sapiens
  source 1..20: mol_type = genomic DNA
agcctggcttgtagtctctg

SEQ ID NO: 42 - DNA, 20 bp, Homo sapiens
  source 1..20: mol_type = genomic DNA
ggcttgtagtctctgcccag

SEQ ID NO: 43 - DNA, 20 bp, Homo sapiens
  source 1..20: mol_type = genomic DNA
ggcaggcactggcacgtgta

SEQ ID NO: 44 - DNA, 20 bp, Homo sapiens
  source 1..20: mol_type = genomic DNA
tggagacttgtgacggtaca

SEQ ID NO: 45 - DNA, 20 bp, Homo sapiens
  source 1..20: mol_type = genomic DNA
catggagacttgtgacggta

SEQ ID NO: 46 - DNA, 20 bp, Homo sapiens
  source 1..20: mol_type = genomic DNA
agaaagacgagaaagacgcg

SEQ ID NO: 47 - DNA, 20 bp, Homo sapiens
  source 1..20: mol_type = genomic DNA
tggttccagctgagctgccg

SEQ ID NO: 48 - DNA, 80 bp, Homo sapiens
  source 1..80: mol_type = genomic DNA
cctggatggtgttattggccaagcctggcttgtagtctctgcccaggtcctggactttgtcgaagaatcgggaaagcgtc

SEQ ID NO: 49 - DNA, 20 bp, Homo sapiens
  source 1..20: mol_type = genomic DNA
acaggcgctggatgagctgc

SEQ ID NO: 50 - DNA, 249 bp, Homo sapiens
  source 1..249: mol_type = genomic DNA
cttcatgcgggccctctggcccatggctcactaggtctgtgtccacatccctccagagcatcttacaggcgctggatgagctgctggaggcccctggggacctggagaccctgccccgcttacagcagcactgtgtggccagtcacctgctggatggcctagaggatgtcctcagagcctgagcaagaacctttccaatgggctgttgaacttcagttatcctgcaggcacaggtaggtccctgggtct

SEQ ID NO: 51 - DNA, 249 bp, Homo sapiens
  source 1..249: mol_type = genomic DNA
agacccagggacctacctgtgcctgcaggataactgaagttcaacagcccattggaaaggttcttgctcaggctctgaggacatcctctaggccatccagcaggtgactggccacacagtgctgctgtaagcggggcagggtctccaggtccccaggggcctccagcagctcatccagcgcctgtaagatgctctggagggatgtggacacagacctagtgagccatgggccagagggcccgcatgaag

SEQ ID NO: 52 - DNA, 20 bp, Homo sapiens
  source 1..20: mol_type = genomic DNA
gcagctcatccagcgcctgt

SEQ ID NO: 53 - DNA, 81 bp, synthetic construct
  source 1..81: mol_type = other DNA
gacgctttcccgattcttcgacaaagtccaggacctgggcagagactacaagccaggcttggccaataacaccaatccagg

SEQ ID NO: 54 - DNA, 81 bp, synthetic construct
  source 1..81: mol_type = other DNA
cctggattggtgttattggccaagcctggcttgtagtctctgcccaggtcctggactttgtcgaagaatcgggaaagcgtc

SEQ ID NO: 55 - protein, 26 aa, synthetic construct
TLSRFFDKVQDLGRDYKPGLANNTNP

SEQ ID NO: 56 - DNA, 250 bp, synthetic construct
  source 1..250: mol_type = other DNA
tcttcatgcgggccctctggcccatggctcactaggtctgtgtccacatccctccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacaggtaggtccctgggtc

SEQ ID NO: 57 - DNA, 250 bp, synthetic construct
  source 1..250: mol_type = other DNA
gacccagggacctacctgtgcctgcaggataactgaagttcaacagcccattggaaaggttcttgctcaggcctctgaggacatcctctaggccatccagcaggtgactggccacacagtgctgctgtaagcggggcagggtctccaggtccccaggggcctccagcagctcatccagcgcctgtaagatgctctggagggatgtggacacagacctagtgagccatgggccagagggcccgcatgaaga

SEQ ID NO: 58 - protein, 59 aa, synthetic construct
EHLTGAGAAGGPWGPGDPAPLTAALCGQSPAGWPRGCPQRPEQEPFQWAVELQLSCRHR

SEQ ID NO: 59 - RNA, 20 nt, synthetic construct
  source 1..20: mol_type = other RNA
cgggctggcaaccgattccg

SEQ ID NO: 60 - RNA, 20 nt, synthetic construct
  source 1..20: mol_type = other RNA
ccgattccggggtcccccaa

SEQ ID NO: 61 - RNA, 20 nt, synthetic construct
  source 1..20: mol_type = other RNA
cggaatcccgaataaccaaa

SEQ ID NO: 62 - RNA, 20 nt, synthetic construct
  source 1..20: mol_type = other RNA
cccggaatcggttgccagcc

SEQ ID NO: 63 - RNA, 20 nt, synthetic construct
  source 1..20: mol_type = other RNA
gttattcgggattccgtgtc

SEQ ID NO: 64 - RNA, 20 nt, synthetic construct
  source 1..20: mol_type = other RNA
gatgcagctcgactggaatc

SEQ ID NO: 65 - RNA, 20 nt, synthetic construct
  source 1..20: mol_type = other RNA
ccagcgcctgtaagatgctc

SEQ ID NO: 66 - RNA, 20 nt, synthetic construct
  source 1..20: mol_type = other RNA
ccagagcatcttacaggcgc

SEQ ID NO: 67 - RNA, 20 nt, synthetic construct
  source 1..20: mol_type = other RNA
ccttacctggatggtgttat

SEQ ID NO: 68 - RNA, 20 nt, synthetic construct
  source 1..20: mol_type = other RNA
ccaataacaccatccaggta

SEQ ID NO: 69 - RNA, 20 nt, synthetic construct
  source 1..20: mol_type = other RNA
cttggccaataacaccatcc

SEQ ID NO: 70 - RNA, 20 nt, synthetic construct
  source 1..20: mol_type = other RNA
cagagactacaagccaggct

SEQ ID NO: 71 - RNA, 20 nt, synthetic construct
  source 1..20: mol_type = other RNA
ctgggcagagactacaagcc

SEQ ID NO: 72 - RNA, 20 nt, synthetic construct
  source 1..20: mol_type = other RNA
tacacgtgccagtgcctgcc

SEQ ID NO: 73 - RNA, 20 nt, synthetic construct
  source 1..20: mol_type = other RNA
tgtaccgtcacaagtctcca

SEQ ID NO: 74 - RNA, 20 nt, synthetic construct
  source 1..20: mol_type = other RNA
taccgtcacaagtctccatg

SEQ ID NO: 75 - RNA, 20 nt, synthetic construct
  source 1..20: mol_type = other RNA
cgcgtctttctcgtctttct

SEQ ID NO: 76 - RNA, 20 nt, synthetic construct
  source 1..20: mol_type = other RNA
cggcagctcagctggaacca

SEQ ID NO: 77 - DNA, 20 bp, Homo sapiens
  source 1..20: mol_type = genomic DNA
ctatcagtgccgctgccgcc

SEQ ID NO: 78 - DNA, 20 bp, Homo sapiens
  source 1..20: mol_type = genomic DNA
tatcagtgccgctgccgccc

SEQ ID NO: 79 - DNA, 20 bp, Homo sapiens
  source 1..20: mol_type = genomic DNA
gttgccagcccgggcggcag

SEQ ID NO: 80 - DNA, 20 bp, Homo sapiens
  source 1..20: mol_type = genomic DNA
gcttccagcctgggcggcag

SEQ ID NO: 81 - DNA, 20 bp, Homo sapiens
  source 1..20: mol_type = genomic DNA
ccgcttacagcagcactgtg

SEQ ID NO: 82 - DNA, 20 bp, Homo sapiens
  source 1..20: mol_type = genomic DNA
gatgcagctcgactggaatc

SEQ ID NO: 83 - DNA, 20 bp, Homo sapiens
  source 1..20: mol_type = genomic DNA
tggaatcaggcacagaaatc

SEQ ID NO: 84 - DNA, 20 bp, Homo sapiens
  source 1..20: mol_type = genomic DNA
aggtgcagaagcaagtagac

SEQ ID NO: 85 - DNA, 20 bp, Homo sapiens
  source 1..20: mol_type = genomic DNA
gcacagaaatctggtgaccc

SEQ ID NO: 86 - DNA, 20 bp, Homo sapiens
  source 1..20: mol_type = genomic DNA
gacacaccagggcttgctgc

SEQ ID NO: 87 - DNA, 20 bp, Homo sapiens
  source 1..20: mol_type = genomic DNA
caccagggcttgctgcagga

SEQ ID NO: 88 - DNA, 20 bp, Homo sapiens
  source 1..20: mol_type = genomic DNA
gatgtgcaggtgagaccctt

SEQ ID NO: 89 - DNA, 20 bp, Homo sapiens
  source 1..20: mol_type = genomic DNA
aattgatcaaaccggacaca

SEQ ID NO: 90 - DNA, 20 bp, Homo sapiens
  source 1..20: mol_type = genomic DNA
gcatccaggtcaaggtggcc

SEQ ID NO: 91 - DNA, 20 bp, Homo sapiens
  source 1..20: mol_type = genomic DNA
cacatctgaggacaggaaga

SEQ ID NO: 92 - DNA, 20 bp, Homo sapiens
  source 1..20: mol_type = genomic DNA
acatctgcaagaggaaggag

SEQ ID NO: 93 - DNA, 20 bp, Homo sapiens
  source 1..20: mol_type = genomic DNA
acatctgaggacaggaagaa

SEQ ID NO: 94 - DNA, 20 bp, Homo sapiens
  source 1..20: mol_type = genomic DNA
tgtctggaacacaacaggac

SEQ ID NO: 95 - DNA, 20 bp, Homo sapiens
  source 1..20: mol_type = genomic DNA
catctgaggacaggaagaag

SEQ ID NO: 96 - DNA, 20 bp, Homo sapiens
  source 1..20: mol_type = genomic DNA
atctgaggacaggaagaagg

SEQ ID NO: 97 - DNA, 20 bp, Homo sapiens
  source 1..20: mol_type = genomic DNA
gggcctgcagggcgaggcca

SEQ ID NO: 98 - DNA, 20 bp, Homo sapiens
  source 1..20: mol_type = genomic DNA
taagatgctctggagggatg

SEQ ID NO: 99 - DNA, 20 bp, Homo sapiens
  source 1..20: mol_type = genomic DNA
tgactgcaggaacagaacac

SEQ ID NO: 100 - DNA, 20 bp, Homo sapiens
  source 1..20: mol_type = genomic DNA
acatctgaggacaaagccag

SEQ ID NO: 101 - DNA, 20 bp, Homo sapiens
  source 1..20: mol_type = genomic DNA
ggcggcagcggcactgatag

SEQ ID NO: 102 - DNA, 20 bp, Homo sapiens
  source 1..20: mol_type = genomic DNA
gggcggcagcggcactgata

SEQ ID NO: 103 - DNA, 20 bp, Homo sapiens
  source 1..20: mol_type = genomic DNA
ctgccgcccgggctggcaac

SEQ ID NO: 104 - DNA, 20 bp, Homo sapiens
  source 1..20: mol_type = genomic DNA
ctgccgcccaggctggaagc

SEQ ID NO: 105 - DNA, 20 bp, Homo sapiens
  source 1..20: mol_type = genomic DNA
cacagtgctgctgtaagcgg

SEQ ID NO: 106 - DNA, 20 bp, Homo sapiens
  source 1..20: mol_type = genomic DNA
gattccagtcgagctgcatc

SEQ ID NO: 107 - DNA, 20 bp, Homo sapiens
  source 1..20: mol_type = genomic DNA
gatttctgtgcctgattcca

SEQ ID NO: 108 - DNA, 20 bp, Homo sapiens
  source 1..20: mol_type = genomic DNA
gtctacttgcttctgcacct

SEQ ID NO: 109 - DNA, 20 bp, Homo sapiens
  source 1..20: mol_type = genomic DNA
gggtcaccagatttctgtgc

SEQ ID NO: 110 - DNA, 20 bp, Homo sapiens
  source 1..20: mol_type = genomic DNA
gcagcaagccctggtgtgtc

SEQ ID NO: 111 - DNA, 20 bp, Homo sapiens
  source 1..20: mol_type = genomic DNA
tcctgcagcaagccctggtg

SEQ ID NO: 112 - DNA, 20 bp, Homo sapiens
  source 1..20: mol_type = genomic DNA
aagggtctcacctgcacatc

SEQ ID NO: 113 - DNA, 20 bp, Homo sapiens
  source 1..20: mol_type = genomic DNA
tgtgtccggtttgatcaatt

SEQ ID NO: 114 - DNA, 20 bp, Homo sapiens
  source 1..20: mol_type = genomic DNA
ggccaccttgacctggatgc

SEQ ID NO: 115 - DNA, 20 bp, Homo sapiens
  source 1..20: mol_type = genomic DNA
tcttcctgtcctcagatgtg

SEQ ID NO: 116 - DNA, 20 bp, Homo sapiens
  source 1..20: mol_type = genomic DNA
ctccttcctcttgcagatgt

SEQ ID NO: 117 - DNA, 20 bp, Homo sapiens
  source 1..20: mol_type = genomic DNA
ttcttcctgtcctcagatgt

SEQ ID NO: 118 - DNA, 20 bp, Homo sapiens
  source 1..20: mol_type = genomic DNA
gtcctgttgtgttccagaca

SEQ ID NO: 119 - DNA, 20 bp, Homo sapiens
  source 1..20: mol_type = genomic DNA
cttcttcctgtcctcagatg

SEQ ID NO: 120 - DNA, 20 bp, Homo sapiens
  source 1..20: mol_type = genomic DNA
ccttcttcctgtcctcagat

SEQ ID NO: 121 - DNA, 20 bp, Homo sapiens
  source 1..20: mol_type = genomic DNA
tggcctcgccctgcaggccc

SEQ ID NO: 122 - DNA, 20 bp, Homo sapiens
  source 1..20: mol_type = genomic DNA
catccctccagagcatctta

SEQ ID NO: 123 - DNA, 20 bp, Homo sapiens
  source 1..20: mol_type = genomic DNA
gtgttctgttcctgcagtca

SEQ ID NO: 124 - DNA, 20 bp, Homo sapiens
  source 1..20: mol_type = genomic DNA
ctggctttgtcctcagatgt

SEQ ID NO: 125 - RNA, 20 nt, synthetic construct
  source 1..20: mol_type = other RNA
ctatcagtgccgctgccgcc

SEQ ID NO: 126 - RNA, 20 nt, synthetic construct
  source 1..20: mol_type = other RNA
tatcagtgccgctgccgccc

SEQ ID NO: 127 - RNA, 20 nt, synthetic construct
  source 1..20: mol_type = other RNA
gttgccagcccgggcggcag

SEQ ID NO: 128 - RNA, 20 nt, synthetic construct
  source 1..20: mol_type = other RNA
gcttccagcctgggcggcag

SEQ ID NO: 129 - RNA, 20 nt, synthetic construct
  source 1..20: mol_type = other RNA
ccgcttacagcagcactgtg

SEQ ID NO: 130 - DNA, 20 bp, Homo sapiens
  source 1..20: mol_type = genomic DNA
agcactcacacggtgggaga

SEQ ID NO: 131 - RNA, 20 nt, synthetic construct
  source 1..20: mol_type = other RNA
tggaatcaggcacagaaatc

SEQ ID NO: 132 - RNA, 20 nt, synthetic construct
  source 1..20: mol_type = other RNA
aggtgcagaagcaagtagac

SEQ ID NO: 133 - RNA, 20 nt, synthetic construct
  source 1..20: mol_type = other RNA
gcacagaaatctggtgaccc

SEQ ID NO: 134 - RNA, 20 nt, synthetic construct
  source 1..20: mol_type = other RNA
gacacaccagggcttgctgc

SEQ ID NO: 135 - RNA, 20 nt, synthetic construct
  source 1..20: mol_type = other RNA
caccagggcttgctgcagga

SEQ ID NO: 136 - RNA, 20 nt, synthetic construct
  source 1..20: mol_type = other RNA
gatgtgcaggtgagaccctt

SEQ ID NO: 137 - RNA, 20 nt, synthetic construct
  source 1..20: mol_type = other RNA
aattgatcaaaccggacaca

SEQ ID NO: 138 - RNA, 20 nt, synthetic construct
  source 1..20: mol_type = other RNA
gcatccaggtcaaggtggcc

SEQ ID NO: 139 - RNA, 20 nt, synthetic construct
  source 1..20: mol_type = other RNA
cacatctgaggacaggaaga

SEQ ID NO: 140 - RNA, 20 nt, synthetic construct
  source 1..20: mol_type = other RNA
acatctgcaagaggaaggag

SEQ ID NO: 141 - RNA, 20 nt, synthetic construct
  source 1..20: mol_type = other RNA
acatctgaggacaggaagaa

SEQ ID NO: 142 - RNA, 20 nt, synthetic construct
  source 1..20: mol_type = other RNA
tgtctggaacacaacaggac

SEQ ID NO: 143 - RNA, 20 nt, synthetic construct
  source 1..20: mol_type = other RNA
catctgaggacaggaagaag

SEQ ID NO: 144 - RNA, 20 nt, synthetic construct
  source 1..20: mol_type = other RNA
atctgaggacaggaagaagg

SEQ ID NO: 145 - RNA, 20 nt, synthetic construct
  source 1..20: mol_type = other RNA
gggcctgcagggcgaggcca

SEQ ID NO: 146 - RNA, 20 nt, synthetic construct
  source 1..20: mol_type = other RNA
taagatgctctggagggatg

SEQ ID NO: 147 - RNA, 20 nt, synthetic construct
  source 1..20: mol_type = other RNA
tgactgcaggaacagaacac

SEQ ID NO: 148 - RNA, 20 nt, synthetic construct
  source 1..20: mol_type = other RNA
acatctgaggacaaagccag

SEQ ID NO: 149 - DNA, 20 bp, Homo sapiens
  source 1..20: mol_type = genomic DNA
cctctacctgtgcagagctt

SEQ ID NO: 150 - DNA, 20 bp, Homo sapiens
  source 1..20: mol_type = genomic DNA
ctctacctgtgcagagcttc

SEQ ID NO: 151 - DNA, 20 bp, Homo sapiens
  source 1..20: mol_type = genomic DNA
tctcccaccgtgtgagtgct

SEQ ID NO: 152 - DNA, 20 bp, Homo sapiens
  source 1..20: mol_type = genomic DNA
agactcacccctggagtcct

SEQ ID NO: 153 - DNA, 20 bp, Homo sapiens
  source 1..20: mol_type = genomic DNA
atcttaccttgtgttccgga

SEQ ID NO: 154 - DNA, 20 bp, Homo sapiens
  source 1..20: mol_type = genomic DNA
ctcacctggctgtggactcc

SEQ ID NO: 155 - DNA, 20 bp, Homo sapiens
  source 1..20: mol_type = genomic DNA
aagctctgcacaggtagagg

SEQ ID NO: 156 - DNA, 20 bp, Homo sapiens
  source 1..20: mol_type = genomic DNA
gaagctctgcacaggtagag

SEQ ID NO: 157 - DNA, 20 bp, Homo sapiens
  source 1..20: mol_type = genomic DNA
tccggaacacaaggtaagat

SEQ ID NO: 158 - DNA, 20 bp, Homo sapiens
  source 1..20: mol_type = genomic DNA
aggactccaggggtgagtct

SEQ ID NO: 159 - DNA, 20 bp, Homo sapiens
  source 1..20: mol_type = genomic DNA
gtggtacctgctggctgagg

SEQ ID NO: 160 - DNA, 20 bp, Homo sapiens
  source 1..20: mol_type = genomic DNA
ggagtccacagccaggtgag

SEQ ID NO: 161 - RNA, 20 nt, synthetic construct
  source 1..20: mol_type = other RNA
cctctacctgtgcagagctt

SEQ ID NO: 162 - RNA, 20 nt, synthetic construct
  source 1..20: mol_type = other RNA
ctctacctgtgcagagcttc

SEQ ID NO: 163 - DNA, 20 bp, Homo sapiens
  source 1..20: mol_type = genomic DNA
cctcagccagcaggtaccac

SEQ ID NO: 164 - RNA, 20 nt, synthetic construct
  source 1..20: mol_type = other RNA
agactcacccctggagtcct

SEQ ID NO: 165 - RNA, 20 nt, synthetic construct
  source 1..20: mol_type = other RNA
acaggcgctggatgagctgc

SEQ ID NO: 166 - RNA, 20 nt, synthetic construct
  source 1..20: mol_type = other RNA
ctcacctggctgtggactcc

SEQ ID NO: 167 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 168 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 169 - protein, 24 aa, synthetic construct
IYIWAPLAGTCGVLLLSLVITLYC

SEQ ID NO: 170 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 171 - protein, 41 aa, synthetic construct
KRGRKKLLYIFKQPFMRVQTTQEEDGCSCRFPEEEEGGCEL

SEQ ID NO: 172 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 173 - protein, 1830 aa, synthetic construct
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLPKKKRKV

SEQ ID NO: 174 - DNA, 5493 bp, synthetic construct
  source 1..5493: mol_type = other DNA
atgacctctgagaagggccctagcacaggcgaccccaccctgcggcggagaatcgagagctgggagttcgacgtgttctacgaccctagagaactgagaaaggaaacctgcctgctgtacgagatcaagtggggcatgagcagaaagatctggcggagctctggcaagaacaccaccaaccacgtggaagtgaatttcatcaagaagttcaccagcgagagaaggttccacagcagcatcagctgcagcatcacctggttcctgagctggtccccttgctgggaatgcagccaggccatcagagagttcctgagccaacaccccggagtgacactggtgatctacgtggccagactgttctggcacatggaccagagaaacagacagggcctgagagatctggtcaacagcggcgtgactatccagatcatgcgggccagcgagtactaccactgttggcggaacttcgtgaactacccccccggcgatgaggcccactggcctcagtaccctcctctgtggatgatgctgtacgccctggaactgcactgcatcatcctgtctctgcctccatgtctgaagatctctagaagatggcagaaccacctggccttcttcagactgcacctgcagaattgccactaccagaccatccccccccacatcctgctggctacaggcctgatccacccttctgtgacctggagacttaagagcggaggatctagcggcggctctagcggatctgagacacctggcacaagcgagtctgccacacctgagagtagcggcggatcttctggtggctctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggtggaagcggaggatctggcggcagcaccaatctgagcgacatcatcgagaaagagacaggcaagcagctggtcatccaagagtccatcctgatgctgcctgaagaggtggaagaagtgatcggcaacaagcccgagtccgacatcctggtgcacaccgcctacgatgagagcaccgacgagaacgtgatgctgctgacctctgacgcccctgagtacaagccttgggctctcgtgatccaggacagcaacggcgagaacaagatcaagatgctgagcggcggctctggtggctctggcggatctacaaacctgtccgatattattgagaaagaaaccgggaaacagctcgtgattcaagagtctattctcatgctcccggaagaagtcgaggaagtcattggaaacaagcctgagagcgatattctggtccatacagcctacgacgagtctaccgatgagaatgtcatgctcctcaccagcgacgctcccgagtataagccatgggcacttgtcattcaggactccaatggggaaaacaaaatcaaaatgctcccaaagaaaaaacgcaaggtgtag

SEQ ID NO: 175 - protein, 1588 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 176 - DNA, 4767 bp, synthetic construct
  source 1..4767: mol_type = other DNA
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtctag

SEQ ID NO: 177 - DNA, 20 bp, Homo sapiens
  source 1..20: mol_type = genomic DNA
tgtagtctctgcccaggtcc

SEQ ID NO: 178 - DNA, 20 bp, Homo sapiens
  source 1..20: mol_type = genomic DNA
ggacctgggcagagactaca

SEQ ID NO: 179 - RNA, 20 nt, synthetic construct
  source 1..20: mol_type = other RNA
tgtagtctctgcccaggtcc

SEQ ID NO: 180 - RNA, 20 nt, synthetic construct
  source 1..20: mol_type = other RNA
gcgcctgtaagatgctctgg

SEQ ID NO: 181 - DNA, 20 bp, Homo sapiens
  source 1..20: mol_type = genomic DNA
ggcgctggatgagctgctgg

SEQ ID NO: 182 - DNA, 20 bp, Homo sapiens
  source 1..20: mol_type = genomic DNA
ccagcagctcatccagcgcc

SEQ ID NO: 183 - RNA, 20 nt, synthetic construct
  source 1..20: mol_type = other RNA
ggcgctggatgagctgctgg

SEQ ID NO: 184 - DNA, 20 bp, Homo sapiens
  source 1..20: mol_type = genomic DNA
cgcctgtaagatgctctgga

SEQ ID NO: 185 - DNA, 20 bp, Homo sapiens
  source 1..20: mol_type = genomic DNA
tccagagcatcttacaggcg

SEQ ID NO: 186 - RNA, 20 nt, synthetic construct
  source 1..20: mol_type = other RNA
cgcctgtaagatgctctgga

SEQ ID NO: 187 - DNA, 20 bp, Homo sapiens
  source 1..20: mol_type = genomic DNA
aacaccatccaggtaaggac

SEQ ID NO: 188 - DNA, 20 bp, Homo sapiens
  source 1..20: mol_type = genomic DNA
gtccttacctggatggtgtt

SEQ ID NO: 189 - RNA, 20 nt, synthetic construct
  source 1..20: mol_type = other RNA
aacaccatccaggtaaggac

SEQ ID NO: 190 - DNA, 20 bp, Homo sapiens
  source 1..20: mol_type = genomic DNA
gagactacaagccaggcttg

SEQ ID NO: 191 - DNA, 20 bp, Homo sapiens
  source 1..20: mol_type = genomic DNA
caagcctggcttgtagtctc

SEQ ID NO: 192 - RNA, 20 nt, synthetic construct
  source 1..20: mol_type = other RNA
gagactacaagccaggcttg

SEQ ID NO: 193 - DNA, 20 bp, Homo sapiens
  source 1..20: mol_type = genomic DNA
caggcttggccaataacacc

SEQ ID NO: 194 - DNA, 20 bp, Homo sapiens
  source 1..20: mol_type = genomic DNA
ggtgttattggccaagcctg

SEQ ID NO: 195 - RNA, 20 nt, synthetic construct
  source 1..20: mol_type = other RNA
caggcttggccaataacacc

SEQ ID NO: 196 - RNA, 20 nt, synthetic construct
  source 1..20: mol_type = other RNA
tccagagcatcttacaggcg

SEQ ID NO: 197 - DNA, 20 bp, Homo sapiens
  source 1..20: mol_type = genomic DNA
ccaggtaaggacaggaccca

SEQ ID NO: 198 - DNA, 20 bp, Homo sapiens
  source 1..20: mol_type = genomic DNA
actgtgtggccagtcacctg

SEQ ID NO: 199 - DNA, 20 bp, Homo sapiens
  source 1..20: mol_type = genomic DNA
tcttacaggcgctggatgag

SEQ ID NO: 200 - DNA, 20 bp, Homo sapiens
  source 1..20: mol_type = genomic DNA
ctcatccagcgcctgtaaga

SEQ ID NO: 201 - RNA, 20 nt, synthetic construct
  source 1..20: mol_type = other RNA
tcttacaggcgctggatgag

SEQ ID NO: 202 - DNA, 20 bp, Homo sapiens
  source 1..20: mol_type = genomic DNA
agctgctggaggcccctggg

SEQ ID NO: 203 - DNA, 20 bp, Homo sapiens
  source 1..20: mol_type = genomic DNA
cccaggggcctccagcagct

SEQ ID NO: 204 - RNA, 20 nt, synthetic construct
  source 1..20: mol_type = other RNA
agctgctggaggcccctggg

SEQ ID NO: 205 - DNA, 20 bp, Homo sapiens
  source 1..20: mol_type = genomic DNA
gggacctggagaccctgccc

SEQ ID NO: 206 - DNA, 20 bp, Homo sapiens
  source 1..20: mol_type = genomic DNA
gggcagggtctccaggtccc

SEQ ID NO: 207 - RNA, 20 nt, synthetic construct
  source 1..20: mol_type = other RNA
gggacctggagaccctgccc

SEQ ID NO: 208 - DNA, 20 bp, Homo sapiens
  source 1..20: mol_type = genomic DNA
tggagaccctgccccgctta

SEQ ID NO: 209 - DNA, 20 bp, Homo sapiens
  source 1..20: mol_type = genomic DNA
taagcggggcagggtctcca

SEQ ID NO: 210 - RNA, 20 nt, synthetic construct
  source 1..20: mol_type = other RNA
tggagaccctgccccgctta

SEQ ID NO: 211 - DNA, 20 bp, Homo sapiens
  source 1..20: mol_type = genomic DNA
ggcttggccaataacaccat

SEQ ID NO: 212 - DNA, 20 bp, Homo sapiens
  source 1..20: mol_type = genomic DNA
atggtgttattggccaagcc

SEQ ID NO: 213 - RNA, 20 nt, synthetic construct
  source 1..20: mol_type = other RNA
ggcttggccaataacaccat

SEQ ID NO: 214 - DNA, 20 bp, Homo sapiens
  source 1..20: mol_type = genomic DNA
tgagctgctggaggcccctg

SEQ ID NO: 215 - DNA, 20 bp, Homo sapiens
  source 1..20: mol_type = genomic DNA
caggggcctccagcagctca

SEQ ID NO: 216 - RNA, 20 nt, synthetic construct
  source 1..20: mol_type = other RNA
tgagctgctggaggcccctg

SEQ ID NO: 217 - DNA, 20 bp, Homo sapiens
  source 1..20: mol_type = genomic DNA
ctgtgtggccagtcacctgc

SEQ ID NO: 218 - DNA, 20 bp, Homo sapiens
  source 1..20: mol_type = genomic DNA
gcaggtgactggccacacag

SEQ ID NO: 219 - RNA, 20 nt, synthetic construct
  source 1..20: mol_type = other RNA
ctgtgtggccagtcacctgc

SEQ ID NO: 220 - DNA, 20 bp, Homo sapiens
  source 1..20: mol_type = genomic DNA
agtcacctgctggatggcct

SEQ ID NO: 221 - DNA, 20 bp, Homo sapiens
  source 1..20: mol_type = genomic DNA
aggccatccagcaggtgact

SEQ ID NO: 222 - RNA, 20 nt, synthetic construct
  source 1..20: mol_type = other RNA
agtcacctgctggatggcct

SEQ ID NO: 223 - DNA, 20 bp, Homo sapiens
  source 1..20: mol_type = genomic DNA
agaggatgtcctcagaggcc

SEQ ID NO: 224 - DNA, 20 bp, Homo sapiens
  source 1..20: mol_type = genomic DNA
ggcctctgaggacatcctct

SEQ ID NO: 225 - RNA, 20 nt, synthetic construct
  source 1..20: mol_type = other RNA
agaggatgtcctcagaggcc

SEQ ID NO: 226 - DNA, 20 bp, Homo sapiens
  source 1..20: mol_type = genomic DNA
caataacaccatccaggtaa

SEQ ID NO: 227 - DNA, 20 bp, Homo sapiens
  source 1..20: mol_type = genomic DNA
ttacctggatggtgttattg

SEQ ID NO: 228 - RNA, 20 nt, synthetic construct
  source 1..20: mol_type = other RNA
caataacaccatccaggtaa

SEQ ID NO: 229 - DNA, 20 bp, Homo sapiens
  source 1..20: mol_type = genomic DNA
cagagcatcttacaggcgct

SEQ ID NO: 230 - DNA, 20 bp, Homo sapiens
  source 1..20: mol_type = genomic DNA
agcgcctgtaagatgctctg

SEQ ID NO: 231 - RNA, 20 nt, synthetic construct
  source 1..20: mol_type = other RNA
cagagcatcttacaggcgct

SEQ ID NO: 232 - DNA, 20 bp, Homo sapiens
  source 1..20: mol_type = genomic DNA
caggcgctggatgagctgct

SEQ ID NO: 233 - DNA, 20 bp, Homo sapiens
  source 1..20: mol_type = genomic DNA
agcagctcatccagcgcctg

SEQ ID NO: 234 - RNA, 20 nt, synthetic construct
  source 1..20: mol_type = other RNA
caggcgctggatgagctgct

SEQ ID NO: 235 - DNA, 20 bp, Homo sapiens
  source 1..20: mol_type = genomic DNA
tgtgtggccagtcacctgct

SEQ ID NO: 236 - DNA, 20 bp, Homo sapiens
  source 1..20: mol_type = genomic DNA
agcaggtgactggccacaca

SEQ ID NO: 237 - RNA, 20 nt, synthetic construct
  source 1..20: mol_type = other RNA
tgtgtggccagtcacctgct

SEQ ID NO: 238 - DNA, 20 bp, Homo sapiens
  source 1..20: mol_type = genomic DNA
agcatcttacaggcgctgga

SEQ ID NO: 239 - DNA, 20 bp, Homo sapiens
  source 1..20: mol_type = genomic DNA
tccagcgcctgtaagatgct

SEQ ID NO: 240 - RNA, 20 nt, synthetic construct
  source 1..20: mol_type = other RNA
agcatcttacaggcgctgga

SEQ ID NO: 241 - DNA, 20 bp, Homo sapiens
  source 1..20: mol_type = genomic DNA
ggcagggtctccaggtcccc

SEQ ID NO: 242 - DNA, 20 bp, Homo sapiens
  source 1..20: mol_type = genomic DNA
ggggacctggagaccctgcc

SEQ ID NO: 243 - RNA, 20 nt, synthetic construct
  source 1..20: mol_type = other RNA
ggcagggtctccaggtcccc

SEQ ID NO: 244 - DNA, 20 bp, Homo sapiens
  source 1..20: mol_type = genomic DNA
gcagggtctccaggtcccca

SEQ ID NO: 245 - DNA, 20 bp, Homo sapiens
  source 1..20: mol_type = genomic DNA
tggggacctggagaccctgc

SEQ ID NO: 246 - RNA, 20 nt, synthetic construct
  source 1..20: mol_type = other RNA
gcagggtctccaggtcccca

SEQ ID NO: 247 - DNA, 20 bp, Homo sapiens
  source 1..20: mol_type = genomic DNA
gctggaggcccctggggacc

SEQ ID NO: 248 - DNA, 20 bp, Homo sapiens
  source 1..20: mol_type = genomic DNA
ggtccccaggggcctccagc

SEQ ID NO: 249 - RNA, 20 nt, synthetic construct
  source 1..20: mol_type = other RNA
gctggaggcccctggggacc

SEQ ID NO: 250 - DNA, 20 bp, Homo sapiens
  source 1..20: mol_type = genomic DNA
atgagctgctggaggcccct

SEQ ID NO: 251 - DNA, 20 bp, Homo sapiens
  source 1..20: mol_type = genomic DNA
aggggcctccagcagctcat

SEQ ID NO: 252 - RNA, 20 nt, synthetic construct
  source 1..20: mol_type = other RNA
atgagctgctggaggcccct

SEQ ID NO: 253 - DNA, 20 bp, Homo sapiens
  source 1..20: mol_type = genomic DNA
gatgagctgctggaggcccc

SEQ ID NO: 254 - DNA, 20 bp, Homo sapiens
  source 1..20: mol_type = genomic DNA
ggggcctccagcagctcatc

SEQ ID NO: 255 - RNA, 20 nt, synthetic construct
  source 1..20: mol_type = other RNA
gatgagctgctggaggcccc

SEQ ID NO: 256 - RNA, 20 nt, synthetic construct
  source 1..20: mol_type = other RNA
atcttaccttgtgttccgga

SEQ ID NO: 257 - RNA, 20 nt, synthetic construct
  source 1..20: mol_type = other RNA
gtggtacctgctggctgagg

SEQ ID NO: 258 - RNA, 20 nt, synthetic construct
  source 1..20: mol_type = other RNA
tccaggtaaggacaggaccc

SEQ ID NO: 259 - RNA, 20 nt, synthetic construct
  source 1..20: mol_type = other RNA
agcactcacacggtgggaga

SEQ ID NO: 260 - DNA, 20 bp, Homo sapiens
  source 1..20: mol_type = genomic DNA
ctctctggattttgaaaaac

SEQ ID NO: 261 - DNA, 20 bp, Homo sapiens
  source 1..20: mol_type = genomic DNA
tgggtcctgtccttacctgg

SEQ ID NO: 262 - DNA, 20 bp, Homo sapiens
  source 1..20: mol_type = genomic DNA
gggtcctgtccttacctgga

SEQ ID NO: 263 - DNA, 20 bp, Homo sapiens
  source 1..20: mol_type = genomic DNA
tccaggtaaggacaggaccc

SEQ ID NO: 264 - RNA, 20 nt, synthetic construct
  source 1..20: mol_type = other RNA
ccaggtaaggacaggaccca

SEQ ID NO: 265 - DNA, 20 bp, Homo sapiens
  source 1..20: mol_type = genomic DNA
cccactacgatgtgcaggtg

SEQ ID NO: 266 - DNA, 20 bp, Homo sapiens
  source 1..20: mol_type = genomic DNA
acaagccaggcttggccaat

SEQ ID NO: 267 - DNA, 20 bp, Homo sapiens
  source 1..20: mol_type = genomic DNA
attggccaagcctggcttgt

SEQ ID NO: 268 - RNA, 20 nt, synthetic construct
  source 1..20: mol_type = other RNA
acaagccaggcttggccaat

SEQ ID NO: 269 - DNA, 20 bp, Homo sapiens
  source 1..20: mol_type = genomic DNA
agccaggcttggccaataac

SEQ ID NO: 270 - DNA, 20 bp, Homo sapiens
  source 1..20: mol_type = genomic DNA
gttattggccaagcctggct

SEQ ID NO: 271 - RNA, 20 nt, synthetic construct
  source 1..20: mol_type = other RNA
agccaggcttggccaataac

SEQ ID NO: 272 - DNA, 20 bp, Homo sapiens
  source 1..20: mol_type = genomic DNA
tggccaataacaccatccag

SEQ ID NO: 273 - DNA, 20 bp, Homo sapiens
  source 1..20: mol_type = genomic DNA
ctggatggtgttattggcca

SEQ ID NO: 274 - RNA, 20 nt, synthetic construct
  source 1..20: mol_type = other RNA
tggccaataacaccatccag

SEQ ID NO: 275 - DNA, 20 bp, Homo sapiens
  source 1..20: mol_type = genomic DNA
agagcatcttacaggcgctg

SEQ ID NO: 276 - DNA, 20 bp, Homo sapiens
  source 1..20: mol_type = genomic DNA
cagcgcctgtaagatgctct

SEQ ID NO: 277 - RNA, 20 nt, synthetic construct
  source 1..20: mol_type = other RNA
agagcatcttacaggcgctg

SEQ ID NO: 278 - DNA, 20 bp, Homo sapiens
  source 1..20: mol_type = genomic DNA
gcatcttacaggcgctggat

SEQ ID NO: 279 - DNA, 20 bp, Homo sapiens
  source 1..20: mol_type = genomic DNA
atccagcgcctgtaagatgc

SEQ ID NO: 280 - RNA, 20 nt, synthetic construct
  source 1..20: mol_type = other RNA
gcatcttacaggcgctggat

SEQ ID NO: 281 - DNA, 20 bp, Homo sapiens
  source 1..20: mol_type = genomic DNA
tacaggcgctggatgagctg

SEQ ID NO: 282 - DNA, 20 bp, Homo sapiens
  source 1..20: mol_type = genomic DNA
cagctcatccagcgcctgta

SEQ ID NO: 283 - RNA, 20 nt, synthetic construct
  source 1..20: mol_type = other RNA
tacaggcgctggatgagctg

SEQ ID NO: 284 - DNA, 20 bp, Homo sapiens
  source 1..20: mol_type = genomic DNA
aggcgctggatgagctgctg

SEQ ID NO: 285 - DNA, 20 bp, Homo sapiens
  source 1..20: mol_type = genomic DNA
cagcagctcatccagcgcct

SEQ ID NO: 286 - RNA, 20 nt, synthetic construct
  source 1..20: mol_type = other RNA
aggcgctggatgagctgctg

SEQ ID NO: 287 - DNA, 20 bp, Homo sapiens
  source 1..20: mol_type = genomic DNA
agcactgtgtggccagtcac

SEQ ID NO: 288 - DNA, 20 bp, Homo sapiens
  source 1..20: mol_type = genomic DNA
gtgactggccacacagtgct

SEQ ID NO: 289 - RNA, 20 nt, synthetic construct
  source 1..20: mol_type = other RNA
agcactgtgtggccagtcac

SEQ ID NO: 290 - DNA, 20 bp, Homo sapiens
  source 1..20: mol_type = genomic DNA
caggtgactggccacacagt

SEQ ID NO: 291 - RNA, 20 nt, synthetic construct
  source 1..20: mol_type = other RNA
actgtgtggccagtcacctg

SEQ ID NO: 292 - DNA, 20 bp, Homo sapiens
  source 1..20: mol_type = genomic DNA
tggccagtcacctgctggat

SEQ ID NO: 293 - DNA, 20 bp, Homo sapiens
  source 1..20: mol_type = genomic DNA
atccagcaggtgactggcca

SEQ ID NO: 294 - RNA, 20 nt, synthetic construct
  source 1..20: mol_type = other RNA
tggccagtcacctgctggat

SEQ ID NO: 295 - DNA, 20 bp, Homo sapiens
  source 1..20: mol_type = genomic DNA
ccagtcacctgctggatggc

SEQ ID NO: 296 - DNA, 20 bp, Homo sapiens
  source 1..20: mol_type = genomic DNA
gccatccagcaggtgactgg

SEQ ID NO: 297 - RNA, 20 nt, synthetic construct
  source 1..20: mol_type = other RNA
ccagtcacctgctggatggc

SEQ ID NO: 298 - DNA, 20 bp, Homo sapiens
  source 1..20: mol_type = genomic DNA
tgctggatggcctagaggat

SEQ ID NO: 299 - DNA, 20 bp, Homo sapiens
  source 1..20: mol_type = genomic DNA
atcctctaggccatccagca

SEQ ID NO: 300 - RNA, 20 nt, synthetic construct
  source 1..20: mol_type = other RNA
tgctggatggcctagaggat

SEQ ID NO: 301 - DNA, 20 bp, Homo sapiens
  source 1..20: mol_type = genomic DNA
aggatgtcctcagaggcctg

SEQ ID NO: 302 - DNA, 20 bp, Homo sapiens
  source 1..20: mol_type = genomic DNA
caggcctctgaggacatcct

SEQ ID NO: 303 - RNA, 20 nt, synthetic construct
  source 1..20: mol_type = other RNA
aggatgtcctcagaggcctg

SEQ ID NO: 304 - DNA, 20 bp, Homo sapiens
  source 1..20: mol_type = genomic DNA
gcaagaacctttccaatggg

SEQ ID NO: 305 - DNA, 20 bp, Homo sapiens
  source 1..20: mol_type = genomic DNA
cccattggaaaggttcttgc

SEQ ID NO: 306 - RNA, 20 nt, synthetic construct
  source 1..20: mol_type = other RNA
gcaagaacctttccaatggg

SEQ ID NO: 307 - DNA, 20 bp, Homo sapiens
  source 1..20: mol_type = genomic DNA
aatgtcagcagaacccaagg

SEQ ID NO: 308 - DNA, 20 bp, Homo sapiens
  source 1..20: mol_type = genomic DNA
ccttgggttctgctgacatt

SEQ ID NO: 309 - RNA, 20 nt, synthetic construct
  source 1..20: mol_type = other RNA
aatgtcagcagaacccaagg

SEQ ID NO: 310 - DNA, 20 bp, Homo sapiens
  source 1..20: mol_type = genomic DNA
cgtgccagtgcctgcctggc

SEQ ID NO: 311 - DNA, 20 bp, Homo sapiens
  source 1..20: mol_type = genomic DNA
gccaggcaggcactggcacg

SEQ ID NO: 312 - RNA, 20 nt, synthetic construct
  source 1..20: mol_type = other RNA
cgtgccagtgcctgcctggc

SEQ ID NO: 313 - DNA, 20 bp, Homo sapiens
  source 1..20: mol_type = genomic DNA
gtaaaagctacggcacctgc

SEQ ID NO: 314 - DNA, 20 bp, Homo sapiens
  source 1..20: mol_type = genomic DNA
gcaggtgccgtagcttttac

SEQ ID NO: 315 - RNA, 20 nt, synthetic construct
  source 1..20: mol_type = other RNA
gtaaaagctacggcacctgc

SEQ ID NO: 316 - DNA, 20 bp, Homo sapiens
  source 1..20: mol_type = genomic DNA
cacctgcacatcgtagtggg

SEQ ID NO: 317 - RNA, 20 nt, synthetic construct
  source 1..20: mol_type = other RNA
cccactacgatgtgcaggtg

SEQ ID NO: 318 - DNA, 20 bp, Homo sapiens
  source 1..20: mol_type = genomic DNA
gtttttcaaaatccagagag

SEQ ID NO: 319 - RNA, 20 nt, synthetic construct
  source 1..20: mol_type = other RNA
ctctctggattttgaaaaac

SEQ ID NO: 320 - DNA, 20 bp, synthetic construct
  source 1..20: mol_type = other DNA
gccgacgcgaaatcttagcg

SEQ ID NO: 321 - DNA, 20 bp, Homo sapiens
  source 1..20: mol_type = genomic DNA
ggcccactacgatgtgcagg

SEQ ID NO: 322 - DNA, 20 bp, Homo sapiens
  source 1..20: mol_type = genomic DNA
gtctcacctgcacatcgtag

SEQ ID NO: 323 - DNA, 20 bp, Homo sapiens
  source 1..20: mol_type = genomic DNA
tctcacctgcacatcgtagt

SEQ ID NO: 324 - DNA, 20 bp, Homo sapiens
  source 1..20: mol_type = genomic DNA
gagctgctggaggcccctgg

(19) United States
(12) Patent Application Publication
(10) Pub. No.: US 2026/0158063 A1
(43) Pub. Date: Jun. 11, 2026

(54) COMPOSITIONS AND METHODS FOR EMR2 MODIFICATION

(71) Applicant: VOR BIOPHARMA INC., Cambridge, MA (US)

(72) Inventors: John Lydeard, Cambridge, MA (US); Tirtha Chakraborty, Cambridge, MA (US)

(73) Assignee: VOR BIOPHARMA INC., Cambridge, MA (US)

(21) Appl. No.: 18/707,965

(22) PCT Filed: Nov. 9, 2022
(86) PCT No.: PCT/US2022/049460

§ 371 Date: May 7, 2024

Related U.S. Application Data
(60) Provisional application No. 63/277,542, filed on Nov. 9, 2021.

(51) Int. Cl. A61K 35/17; A61K 40/11; A61K 40/31; A61K 40/42; C07K 16/28; C12N 5/0783; C12N 9/22; C12N 15/11; C12N 15/90

(52) U.S. Cl. CPC A61K 35/17; A61K 40/11; A61K 40/31; A61K 40/4224; C07K 16/2896; C12N 5/0636; C12N 9/226; C12N 15/111; C12N 15/907; A61K 2239/13; A61K 2239/48; C12N 2310/20; C12N 2310/315; C12N 2310/321; C12N 2510/00

(57) ABSTRACT

Provided herein are gRNA comprising a targeting domain that targets EMR2, which may be used, for example, to make modifications in cells. Also provided herein are methods of genetically engineered cell having a modification (e.g., insertion or deletion) in the EMR2 gene and methods involving administering such genetically engineered cells to a subject, such as a subject having a hematopoietic malignancy.

### RELATED APPLICATIONS

The application claims the benefit under 35 U.S.C. 119(e) of U.S. Provisional Application No. 63/277,542 filed Nov. 9, 2021 which is incorporated by reference in its entirety.

### BACKGROUND

When a subject is administered an immunotherapy targeting an antigen associated with a disease or condition, e.g., an anti-cancer CAR-T therapy, the therapy can deplete not only the pathological cells intended to be targeted, but also non-pathological cells that may express the targeted antigen. This “on-target, off-disease” effect has been reported for some CAR-T therapeutics, e.g., those targeting CD19 or CD33. If the targeted antigen is expressed on the surface of cells required for survival of the subject, or on the surface of cells the depletion of which is of significant detriment to the health of the subject, the subject may not be able to receive the immunotherapy, or may have to face severe side effects once administered such a therapy. In other instances, it may be desirable to administer an immunotherapy targeting an antigen that is expressed on the immune effector cells that constitute the immunotherapy, e.g., on the surface of CAR-T cells, which may result in fratricide and render the respective therapeutics ineffective or virtually impossible to produce.

### SUMMARY

Some aspects of this disclosure describe compositions, methods, strategies, and treatment modalities that address the detrimental on-target, off-disease effects of certain immunotherapeutic approaches, e.g., of immunotherapeutics comprising lymphocyte effector cells targeting a specific antigen in a subject in need thereof, such as CAR-T cells or CAR-NK cells.

Aspects of the present disclosure provide guide RNAs (gRNA) comprising a targeting domain comprising a sequence described in Tables 1-8. In some aspects, the gRNA comprises a targeting domain, wherein the targeting domain comprises a sequence of any one of SEQ ID NOs: 59-76, 125-148, 161-166, 179, 180, 183, 184, 186, 189, 192, 195, 201, 204, 207, 210, 213, 216, 219, 222, 225, 228, 231, 234, 237, 249, 243, 246, 249, 252, 254-257, 268, 271, 274, 277, 280, 283, 286, 289, 291, 294, 297, 300, 303, 306, 309, 312, 315, 317, and 219. In some embodiments, the gRNA comprises a first complementarity domain, a linking domain, a second complementarity domain which is complementary to the first complementarity domain, and a proximal domain. In some embodiments, the gRNA is a single guide RNA (sgRNA).

In some embodiments, the gRNA comprises one or more nucleotide residues that are chemically modified. In some embodiments, the gRNA comprises one or more nucleotide residues that comprise a 2′O-methyl moiety. In some embodiments, the gRNA comprises one or more nucleotide residues that comprise a phosphorothioate. In some embodiments, the gRNA comprises one or more nucleotide residues that comprise a thioPACE moiety.

Aspects of the present disclosure provide methods of producing a genetically engineered cell, comprising: providing a cell, and contacting the cell with (i) any of the gRNAs described herein or a gRNA targeting a targeting domain targeted by any of the gRNAs described herein; and (ii) an RNA-guided nuclease that binds the gRNA, thus forming a ribonucleoprotein (RNP) complex under conditions suitable for the gRNA of (i) to form and/or maintain an RNP complex with the RNA-guided nuclease of (ii) and for the RNP complex to bind a target domain in the genome of the cell. In some embodiments, the contacting comprises introducing (i) and (ii) into the cell in the form of a pre-formed ribonucleoprotein (RNP) complex. In some embodiments, the contacting comprises introducing (i) and/or (ii) into the cell in the form of a nucleic acid encoding the gRNA of (i) and/or the RNA-guided nuclease of (ii). In some embodiments, the nucleic acid encoding the gRNA of (i) and/or the RNA-guided nuclease of (ii) is an RNA, preferably an mRNA or an mRNA analog. In some embodiments, the ribonucleoprotein complex is introduced into the cell via electroporation.

In some embodiments, the RNA-guided nuclease is a CRISPR/Cas nuclease. In some embodiments, the CRISPR/Cas nuclease is a Cas9 nuclease. In some embodiments, the CRISPR/Cas nuclease is an S. pyogenes Cas (spCas) nuclease. In some embodiments, the Cas nuclease in an S. aureus Cas (saCas nuclease). In some embodiments, the CRISPR/Cas nuclease is a Cpf1 nuclease.

In some embodiments, the CRISPR/Cas nuclease comprises a mutation that diminishes or abrogates a nuclease activity, e.g., in some embodiments, the CRISPR/Cas nuclease is a nickase or a nuclease-dead CRISPR/Cas nuclease variant.

In some embodiments, the CRISPR/Cas nuclease is fused to an endonuclease domain, e.g., a Fok-I nuclease domain. In some embodiments, the CRISPR/Cas nuclease is fused to a deamination domain, e.g., an APOBEC1 or APOBEC3 deamination domain.

In some embodiments, the CRISPR/Cas nuclease is a base editor. In some embodiments, the CRISPR/Cas nuclease is a cytosine base editor. In some embodiments, the CRISPR/Cas nuclease is an adenine base editor.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte.

Aspects of the present disclosure provide genetically engineered cells obtained by any of the methods described herein. Aspects of the present disclosure provide cell populations comprising the genetically engineered cells described herein.

Aspects of the present disclosure provide cell populations comprising a genetically engineered cell, wherein the genetically engineered cell comprises a genomic modification that consists of an insertion or deletion immediately proximal to a site cut by an RNA-guided nuclease when bound to a gRNA comprising a targeting domain as described in Tables 1-8. In some embodiments, wherein the genomic modification is an insertion or deletion generated by a non-homologous end joining (NHEJ) event. In some embodiments, wherein the genomic modification is an insertion or deletion generated by a homology-directed repair (HDR) event. In some embodiments, the genomic modification results in a loss-of function of EMR2 in a genetically engineered cell harboring such a genomic modification. In some embodiments, the genomic modification results in a reduction of expression of EMR2 to less than 25%, less than 20% less than 10% less than 5% less than 2% less than 1%, less than 0.1%, less than 0.01%, or less than 0.001% as compared to the expression level of EMR2 in wild-type cells of the same cell type that do not harbor a genomic modification of EMR2. In some embodiments, the genetically engineered cell is a hematopoietic stem or progenitor cell.

In some embodiments, the genetically engineered cell is an immune effector cell. In some embodiments, the genetically engineered cell is a T-lymphocyte. In some embodiments, the immune effector cell expresses a chimeric antigen receptor (CAR). In some embodiments, the CAR targets EMR2.

In some embodiments, the cell population is characterized by the ability to engraft EMR2-edited hematopoietic stem cells in the bone marrow of a recipient and to generate differentiated progeny of all blood lineage cell types in the recipient. In some embodiments, the cell population is characterized by the ability to engraft EMR2-edited hematopoietic stem cells in the bone marrow of a recipient at an efficiency of at least 50%. In some embodiments, the cell population is characterized by the ability to engraft EMR2-edited hematopoietic stem cells in the bone marrow of a recipient at an efficiency of at least 60%. In some embodiments, the cell population is characterized by the ability to engraft EMR2-edited hematopoietic stem cells in the bone marrow of a recipient at an efficiency of at least 70%. In some embodiments, the cell population is characterized by the ability to engraft EMR2-edited hematopoietic stem cells in the bone marrow of a recipient at an efficiency of at least 80%. In some embodiments, the cell population is characterized by the ability to engraft EMR2-edited hematopoietic stem cells in the bone marrow of a recipient at an efficiency of at least 90%. In some embodiments, the cell population comprises EMR2-edited hematopoietic stem cells that are characterized by a differentiation potential that is equivalent to the differentiation potential of non-edited hematopoietic stem cells.

Aspects of the present disclosure provide methods comprising administering to a subject in need thereof any of the genetically engineered cells described herein or any of the cell populations described herein. In some embodiments, the subject has or has been diagnosed with a hematopoietic malignancy. In some embodiments, the method further comprises administering to the subject an effective amount of an agent that targets EMR2, wherein the agent comprises an antigen-binding fragment that binds EMR2.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A-1E show expression of cell surface markers CD33, CLL-1, CD123, and EMR2 on cells from acute myeloid leukemia patients. FIG. 1A shows the precent positive cells expressing the indicated markers in blasts (CD45^{dim}). FIG. 1B shows the antigen density, as measured by the antibodies bound per cell (blasts). FIG. 1 C shows the precent positive cells expressing the indicated markers in leukemic stem cells (LSCs; CD45^{dim}, CD34+, CD38−). FIG. 1D shows the antigen density, as measured by the antibodies bound per cell (LSCs) of CD33, CLL-1, CD123, and EMR2 in LSCs of AML patients. FIG. 1E shows the percent expression of the indicated markers in bone marrow samples and Leuko Pak samples: hematopoietic stem cells (HSC), multipotent progenitor cells (MPP) cells, common myeloid progenitor (CMP) cells, granulocyte macrophage progenitor (GMP) cells, megakaryocyte erythroid progenitor (MEP) cells, and common lymphoid progenitor (CLP) cells. For each of FIGS. 1A-1D, the average (percent positive or antigen density) for each marker is shown in the tables below the graphs.

FIGS. 2A-2E show an overview of an exemplary CRISPR strategy for EMR2 editing using an exemplary EMR2 gRNAs resulting in insertions/deletions (indels) leading to premature stop codons. FIG. 2A shows exemplary EMR2 gRNAs, the average editing efficiencies and the major indels generated in HL-60 cells or CD34+ cells. FIG. 2B shows exemplary EMR2 gRNAs, the average editing efficiencies, and the major insertion/deletions (indels) generated. FIG. 2C shows a region of the wildtype genomic sequence and amino acid sequence of EMR2. The location of guide EMR2-11 is indicated and the 4 nucleotides deleted in (−4) indel of EMR2-11 is indicated between vertical lines. FIG. 2D shows a region of the genomic sequence and amino acid sequence of EMR2 edited with guide EMR2-11, which generates a −4-deletion resulting in an early stop codon (indicated with a “*”). FIG. 2E shows a region of the wildtype genomic sequence and amino acid sequence of EMR2. The location of guide EMR2-11 is indicated and the nucleotides location of the (+1) indel of EMR2-11 is indicated at the vertical line. FIG. 2F shows a region of the genomic sequence and amino acid sequence EMR2 edited with guide EMR2-11, which generates a +1 insertion resulting in an early stop codon (indicated with a “*”).

FIGS. 3A and 3B show loss of surface expression of EMR2 following electroporation with ribonucleoprotein complexes containing Cas9 and EMR2 targeting gRNAs EMR2-11 or EMR2-18. FIG. 3A shows EMR2 RNA transcript expression in HL-60 cells at 48- and 96-hour time points post-electroporation. For each time point, the bars refer, from left to right, to WT cells, cells editing using guide EMR2-11, and cells edited using guide EMR2-18. FIG. 3B shows EMR2 surface expression as percent EMR2 positive of live cells at 48- and 96-hour time points (2 days and 4 days, respectively) post-electroporation as assessed by flow cytometry.

FIGS. 4A and 4B show the effects of CRISPR editing with the indicated gRNAs targeting EMR2. FIG. 4A shows cell counts and viability at 24 hours, 48 hours, 9 days, and 16 days post electroporation with RNPs containing the indicated gRNAs or mock electroporation (“Mock”). FIG. 4B shows editing efficiency for the indicated gRNAs targeting EMR2, as compared to a CD33 gRNA, control gRNA (gCtrl), or mock electroporation, at 48 hours, 9 days, or 16 days following electroporation, as well as the major indels observed.

FIGS. 5A-5C show insertion/deletion (indel) spectra and editing efficiencies for genomic editing of cells with the indicated EMR2 gRNAs at days 2 (top panels), 9 (middle panels), and 16 (bottom panels) post-electroporation with ribonucleoproteins containing Cas9 and the indicated gRNA. FIG. 5A shows editing with a gRNA targeting CD33 (CD33gRNA). FIG. 5B shows editing with gRNA EMR2-11. FIG. 5C shows editing with gRNA EMR2-18.

FIGS. 6A-6G show surface expression of cell markers following electroporation with ribonucleoprotein complexes containing Cas9 and EMR2 targeting gRNAs EMR2-11 or EMR2-18, a CD33 gRNA, control gRNA (gCtrl), or mock electroporated (“Mock”) over time. FIG. 6A shows EMR2 surface expression shown as the EMR2+ frequency of parent. FIG. 6B shows EMR2 surface expression represented as the fold change over cells electroporated with the control gRNA (gCtrl). FIG. 6C shows CD97 surface expression shown as the frequency of parent. FIG. 6D shows CD33 surface expression shown as the frequency of parent. FIG. 6E shows CD11b surface expression shown as the frequency of parent. FIG. 6F shows CD14 surface expression shown as the frequency of parent. FIG. 6G shows CD15 surface expression shown as the frequency of parent.

FIGS. 7A-7F show cytokine levels from in vitro differentiated CD34+ cells following electroporation with ribonucleoprotein complexes containing Cas9 and EMR2-targeting gRNAs EMR2-11 or EMR2-18, a control gRNA (gCtrl), or mock electroporated (“Mock EP”) and stimulation with lipopolysaccharide (LPS), anti-EMR2 monoclonal antibody 2A1 (2A1), a standard IgG1, or unstimulated (unstim). FIG. 7A shows IFNγ levels. FIG. 7B shows IL-1β levels. FIG. 7C shows IL-6 levels. FIG. 7D shows IL-8 levels. FIG. 7E shows MIP-1a levels. FIG. 7F shows TNFα levels.

FIGS. 8A-8D show cell expansion and viability following electroporation with ribonucleoprotein complexes containing Cas9 and EMR2 targeting gRNAs EMR2-11 or EMR2-18, a gRNA targeting CD33 gRNA (CD33gRNA), a control gRNA (gCtrl), or naïve cells (Naïve). FIG. 8A shows the average number of live cells cultured in granulocytic media (myeloid I media) at the indicated time points post-electroporation. FIG. 8B shows the percent viability of cells cultured in granulocytic media (myeloid I media) at the indicated time points post-electroporation. FIG. 8C shows the average number of live cells cultured in monocytic media (myeloid II media) at the indicated time points post-electroporation. FIG. 8D shows the percent viability of cells cultured in monocytic media (myeloid II media) at the indicated time points post-electroporation.

FIG. 9 shows EMR2 editing persists throughout cell differentiation following electroporation with ribonucleoprotein complexes containing Cas9 and EMR2 targeting gRNAs EMR2-11 or EMR2-18. The percent editing efficiency is shown over time post electroporation in differentiation media (myeloid I media (Mye I) or myeloid II media (Mye II)).

FIGS. 10A and 10B show surface expression of EMR2 over time following electroporation with ribonucleoprotein complexes containing Cas9 and EMR2 targeting gRNAs EMR2-11 or EMR2-18, a guide targeting CD33 (CD33gRNA), a control gRNA (Ctrl), or naïve cells. FIG. 10A shows EMR2 surface expression in cells cultured in granulocytic media (myeloid I media) following electroporation. FIG. 10B shows EMR2 surface expression in cells cultured in in monocytic media (myeloid II media) following electroporation.

FIGS. 11A-11F show expression analyses of monocytic or granulocytic lineage markers following electroporation with ribonucleoprotein complexes containing Cas9 and EMR2 targeting gRNAs EMR2-11 or EMR2-18, a guide targeting CD33 (CD33gRNA), a control gRNA (Ctrl), or naïve cells. FIG. 11A shows expression of CD11b in granulocytic lineage cells when cultured in myeloid I media at the indicated time points. FIG. 11B shows expression of CD14 in granulocytic lineage cells when cultured in myeloid I media at the indicated time points. FIG. 11C shows expression of CD11b in monocytic lineage cells when cultured in myeloid II media at the indicated time points. FIG. 11D shows expression of CD14 in monocytic lineage cells when cultured in myeloid II media at the indicated time points. FIG. 11E shows expression of CD15 in granulocytic lineage cells when cultured in myeloid I media at the indicated time points. FIG. 11F shows expression of CD15 in monocytic lineage cells when cultured in myeloid II media at the indicated time points.

FIGS. 12A-12H show levels of cytokine production following electroporation with ribonucleoprotein complexes containing Cas9 and EMR2 targeting gRNAs EMR2-11 or EMR2-18, or a control gRNA (gCtrl) and stimulation with lipopolysaccharide (LPS stim), R848 (resiquimod, R848 stim), or unstimulated (unstim) at day 14 of differentiation. FIG. 12A shows IL-6 levels in granulocytic lineage cells when cultured in myeloid I media. FIG. 12B shows IL-6 levels in monocytic lineage cells when cultured in myeloid II media. FIG. 12C shows IL-8 levels in granulocytic lineage cells when cultured in myeloid I media. FIG. 12D shows IL-8 levels in monocytic lineage cells when cultured in myeloid II media. FIG. 12E shows MIP-1α levels in granulocytic lineage cells when cultured in myeloid I media. FIG. 12F shows MIP-1α levels in monocytic lineage cells when cultured in myeloid II media. FIG. 12G shows TNFα levels in granulocytic lineage cells when cultured in myeloid I media. FIG. 12H shows TNFα levels in monocytic lineage cells when cultured in myeloid II media.

FIG. 13 shows a schematic of an experimental plan for EMR2 guide screen and protein knockdown assessment.

FIGS. 14A and 14B show expression levels of EMR2 following editing with the indicated EMR2 gRNAs at 6 days post electroporation. FIG. 14A shows flow cytometry analysis of EMR2 protein expression in EMR2-edited HSCs using the indicated gRNAs relative to an isotype control. FIG. 15B shows quantification of the flow cytometry data shown in FIG. 14A.

FIGS. 15A-15C show adenosine base editing (ABE) of EMR2 using the indicated EMR2 targeting gRNAs has no impact on cell viability. FIG. 15A shows cell counts measured on days 0 (D0), 2 (D2), and either day 5 or 6 (D5/D6) following electroporation. FIG. 15B shows cell viability analyses performed on days 0 (D0), 2 (D2), and either day 5 or 6 (D5/D6) following electroporation. For FIGS. 15A and 15B, for each gRNA condition, the bars correspond, from left to right, DO, D2, and D5/D6, respectively.

FIG. 15C is a table showing the number of biological replicates corresponding to the cell count and viability analyses presented in FIGS. 15A and 15B, respectively.

FIGS. 16A-16C show a summary of editing outcomes in EMR2-edited cells. FIG. 16A shows rhAmpSeq analysis of EMR2 editing frequency performed on day 2 (D2) and either 5 or 6 (D5/D6) post electroporation. FIG. 16B shows rhAmpSeq analysis of base editing consequences following editing with the indicated EMR2-targeted gRNA, at either 2 days (D2) or 5 or 6 days (D5/D6) post electroporation. Within each bar of the graph, the bottom portion corresponds to splice site disruption, the middle portion corresponds to missense variants, and the top portion corresponds to indels in EMR2, respectively. FIG. 16C is a table showing the number of biological replicates corresponding to FIGS. 16A and 16B.

FIGS. 17A-17C show a summary of EMR2 surface protein expression on EMR2-edited cells. FIG. 17A shows flow cytometry analyses of EMR2 expression levels in EMR2-edited cells performed on days 0 (D0), 2 (D2), and either day 5 or 6 (D5/D6) post-electroporation represented as the percent EMR2 expression. FIG. 17B shows geometric mean fluorescence intensity (GMFI) flow cytometry analyses of EMR2 expression in EMR2-edited cells performed on days 0 (D0), 2 (D2), and either day 5 or 6 (D5/D6) post-electroporation represented as the geometric mean (gMFI). FIG. 17C is a table showing the number of biological replicates (n) corresponding to the flow cytometry data presented in FIGS. 17A and 17B.

FIGS. 18A and 18B show a summary of droplet digital PCR analyses of EMR2 transcript expression following editing with the indicated EMR2-targeted gRNA or control. Cells were harvested from the experiment on the day of electroporation, 2 days (D2) or 5 days (D5) post electroporation of the indicated EMR2-targeted gRNA, control gRNA (gCrtl) or no electroporation control (no EP), and a single ddPCT reaction was performed with cDNA from all conditions from all time points. For single conditions, gRNAs were used, ABE gRNAs: EMR2-43, EMR2-62, EMR2-63, or EMR2-64. For Triplex conditions, the following gRNAs were used: Triplex 1—EMR2-62, an ABE gRNA targeting CD33, and an ABE gRNA targeting CD123. Triplex 2—ABE gRNA EMR2-64, an ABE gRNA targeting CD33, and an ABE gRNA targeting CD123. FIG. 18A shows the average normalized EMR2 transcript expression using primers for exons 19-20. FIG. 18B shows the average normalized EMR2 transcript expression using primers for exons 10-12. FIG. 18C is a table showing the number of biological replicates (n) corresponding to the EMR transcript data presented in FIGS. 18A and 18B. EMR2 gRNAs EMR2-62, EMR-63, and EMR2-64 resulted in modest EMR2 transcript loss (20-50% loss as compared to no EP).

### DETAILED DESCRIPTION

Some aspects of this disclosure provide compositions, methods, strategies, and treatment modalities related to genetically modified cells, e.g., hematopoietic cells, that are deficient in the expression of an antigen targeted by a therapeutic agent, e.g., an immunotherapeutic agent. The genetically modified cells provided herein are useful, for example, to mitigate, or avoid altogether, certain undesired effects, for example, any on-target, off-disease cytotoxicity, associated with certain immunotherapeutic agents.

Such undesired effects associated with certain immunotherapeutic agents may occur, for example, when healthy cells within a subject in need of an immunotherapeutic intervention express an antigen targeted by an immunotherapeutic agent. For example, a subject may be diagnosed with a malignancy associated with an elevated level of expression of a specific antigen, which is not typically expressed in healthy cells, but may be expressed at relatively low levels in a subset of non-malignant cells within the subject. Administration of an immunotherapeutic agent, e.g., a CAR-T cell therapeutic or a therapeutic antibody or antibody-drug-conjugate (ADC) targeting the antigen, to the subject may result in efficient killing of the malignant cells, but may also result in ablation of non-malignant cells expressing the antigen in the subject. This on-target, off-disease cytotoxicity can result in significant side effects and, in some cases, abrogate the use of an immunotherapeutic agent altogether.

The compositions, methods, strategies, and treatment modalities provided herein address the problem of on-target, off-disease cytotoxicity of certain immunotherapeutic agents. For example, some aspects of this disclosure provide genetically engineered cells harboring a modification in their genome that results in a lack of expression of an antigen, or a specific form of that antigen, targeted by an immunotherapeutic agent. Such genetically engineered cells, and their progeny, are not targeted by the immunotherapeutic agent, and thus not subject to any cytotoxicity effected by the immunotherapeutic agent. Such cells can be administered to a subject receiving an immunotherapeutic agent targeting the antigen, e.g., in order to replace healthy cells that may have been targeted and killed by the cytotherapeutic agent, and/or in order to provide a population of cells that is resistant to targeting by the cytotherapeutic agent. For example, if healthy hematopoietic cells in the subject express the antigen, genetically engineered hematopoietic cells provided herein, e.g., genetically engineered hematopoietic stem or progenitor cells, may be administered to the subject that do not express the antigen, and thus are not targeted by the cytotherapeutic agent. Such hematopoietic stem or progenitor cells are able to re-populate the hematopoietic niche in the subject and their progeny can reconstitute the various hematopoietic lineages, including any that may have been ablated by the cytotherapeutic agent.

EGF-like module-containing mucin-like hormone receptor-like 2 (EMR2), also referred to as CD312, is a 823-amino acid, ˜90 kDa protein (depending on isoform) of the EGF-seven-span transmembrane (TM7) family of adhesion G protein-coupled receptors (GPCR) with a high level of homology with CD97. EMR2 forms a heterodimer and binds to chondroitin sulfate B via its EGF-like domain 4 and mediate cell adhesion, granulocyte chemotaxis, degranulation, and the release of pro-inflammatory cytokines in macrophages. See, e.g. Kuan-Yu et al. Front. Immunol. (2017) 8:373. Without wishing to be bound by any particular theory, EMR2 is expressed on myeloid cells with highest expression in granulocytes, macrophages, and Kupffer cells.

The ADGRE2 gene located on human chromosome 19 encodes human EMR2 and canonically contains 19 exons, although a number of isoforms exist with varying number EGF domains due to alternative RNA splicing. The dominant isoform in whole blood contains 17 exons. See, e.g. Safaee et al. Onc. Rev. (2014). 8(242):20-24.

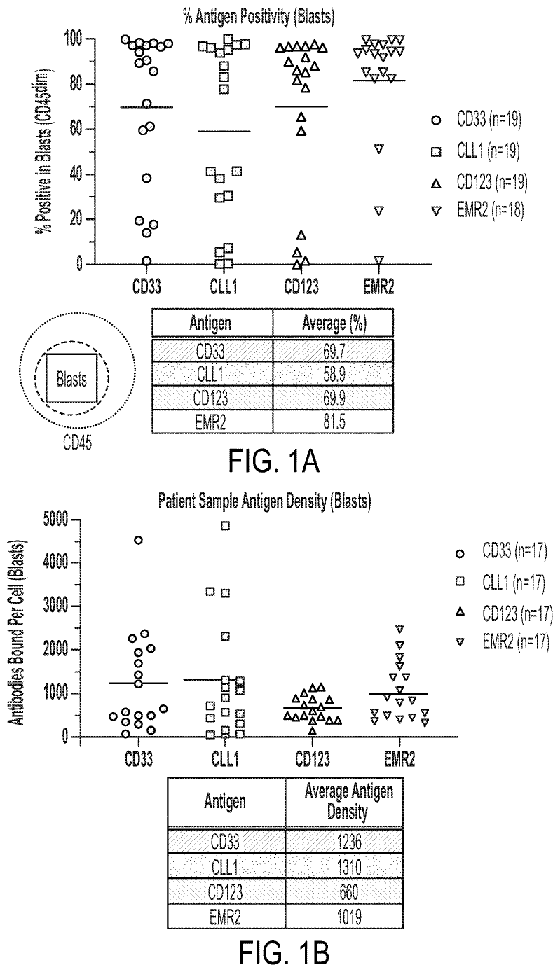

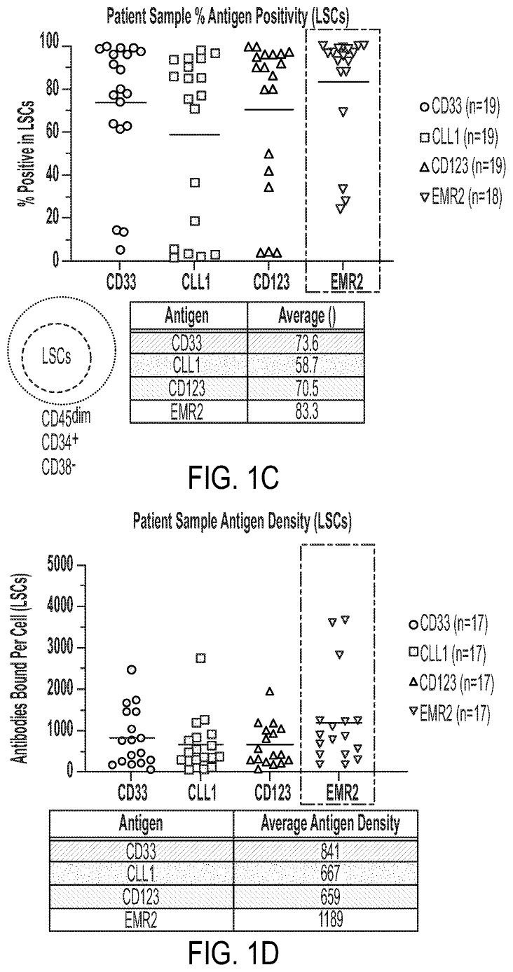

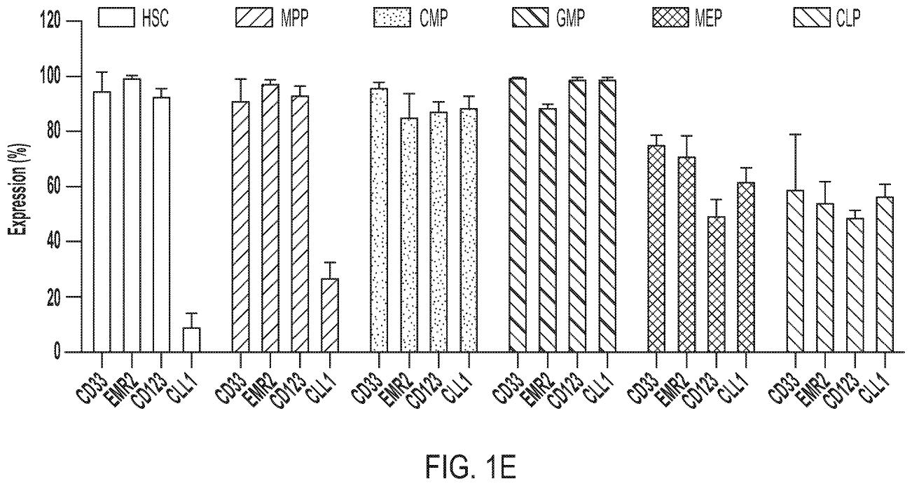

EMR2 expression has also been associated with relapsed/refractory acute myelogenous leukemia (AML). Analysis of target distribution in AML patient cells indicated that EMR2 is expressed on over 90% of AML cells (including leukemic stem cells) in most AML patients, including a majority of relapsed/refractory AML patient samples, along with CD33, CLL1, and CD123. See, FIGS. 1A-1E.

Additionally, aberrant EMR2 expression has been associated with human breast carcinoma and patient survival. See, e.g. Davies et al. Oncol. Rep. (2011) 25(3): 619-627. EMR2 overexpression is associated with other cancers including bladder carcinoma, colorectal carcinoma, gastric and esophageal carcinoma, and glioblastoma. See, e.g. Safaee et al. Oncol. Rev. (2014) 8(242):20-24.

Due to the shared expression of EMR2 on both, activated healthy cells as well as being an expressed antigen on malignant cells, therapeutic targeting of EMR2 may result in killing of heathy cells.

Described herein are gRNAs that have been developed to specifically direct genetic modification of the gene encoding EMR2. Also provided herein is use of such gRNAs to produce genetically modified cells, such as hematopoietic cells, immune cells, lymphocytes, and populations of such cells, that are deficient in EMR2 or have reduced expression of EMR2 such that the modified cells are not recognized by EMR2-specific immunotherapies. Also provided herein are methods involving administering such cells, or compositions thereof, to subjects to address the problem of on-target, off-disease cytotoxicity of certain immunotherapeutic agents. In some examples, as described herein, the genetically modified cells are hematopoietic cells that are deficient in EMR2 or have reduced expression of EMR2 that are capable, for example, of developing into lineage-committed cells, such as T cells that are deficient in EMR2 or have reduced expression of EMR2, or express a mutant or variant of EMR2, and therefore, are resistant to killing by EMR2-specific immunotherapies. Alternatively or in addition, in some examples, as described herein, the genetically modified cells are immune cells, such as EMR2-specific CAR T cells that are deficient in in EMR2, or have reduced expression of EMR2, or express a mutant or variant of EMR2, and therefore, are resistant to fratricide killing by other EMR2-specific CAR T cells.

### Genetically Engineered Cells and Related Compositions and Methods

Some aspects of this disclosure provide genetically engineered cells comprising a modification in their genome that results in a loss of expression of EMR2, or expression of a variant form of EMR2 that is not recognized by an immunotherapeutic agent targeting EMR2. In some embodiments, the modification in the genome of the cell is a mutation in a genomic sequence encoding EMR2.

The term “mutation,” as used herein, refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence. In some embodiments provided herein, a mutation in a gene encoding EMR2 results in a loss of expression of EMR2 in a cell harboring the mutation. In some embodiments, a mutation in a gene encoding EMR2 results in the expression of a variant form of EMR2 that is not bound by an immunotherapeutic agent targeting EMR2, or bound at a significantly lower level than the non-mutated EMR2 form encoded by the gene. In some embodiment, a cell harboring a genomic mutation in the EMR2 gene as provided herein is not bound by, or is bound at a significantly lower level by an immunotherapeutic agent that targets EMR2, e.g., an anti-EMR2 antibody or chimeric antigen receptor (CAR).

Some aspects of this disclosure provide compositions and methods for generating the genetically engineered cells described herein, e.g., genetically engineered cells comprising a modification in their genome that results in a loss of expression of EMR2, or expression of a variant form of EMR2 that is not recognized by an immunotherapeutic agent targeting EMR2. Such compositions and methods provided herein include, without limitation, suitable strategies and approaches for genetically engineering cells, e.g., by using RNA-guided nucleases, such as CRISPR/Cas nucleases, and suitable RNAs able to bind such RNA-guided nucleases and target them to a suitable target site within the genome of a cell to effect a genomic modification resulting in a loss of expression of EMR2, or expression of a variant form of EMR2 that is not recognized by an immunotherapeutic agent targeting EMR2.

In some embodiments, a genetically engineered cell (e.g., a genetically engineered hematopoietic cell, such as, for example, a genetically engineered hematopoietic stem or progenitor cell or a genetically engineered immune effector cell) described herein is generated via genome editing technology, which includes any technology capable of introducing targeted changes, also referred to as “edits,” into the genome of a cell.

One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, e.g., Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzalone et al. Nat. Biotechnol. (2020) 38: 824-844.

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an SpCas9 or an SaCas9 nuclease. For another example, in some embodiments, a suitable RNA-guided nuclease for use in the methods of genetically engineering cells provided herein is a Cas12 nuclease, e.g., a Cas12a nuclease. Exemplary suitable Cas12 nucleases include, without limitation, AsCas12a, FnCas12a, other Cas12a orthologs, and Cas12a derivatives, such as the MAD7 system (MAD7™, Inscripta, Inc.), or the Alt-R Cas12a (Cpf1) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816.

In some embodiments, a genetically engineered cell (e.g., a genetically engineered hematopoietic cell, such as, for example, a genetically engineered hematopoietic stem or progenitor cell or a genetically engineered immune effector cell) described herein is generated by targeting an RNA-guided nuclease, e.g., a CRISPR/Cas nuclease, such as, for example, a Cas9 nuclease or a Cas12a nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs are described in more detail elsewhere herein.

In some embodiments, a EMR2 gRNA described herein is complexed with a CRISPR/Cas nuclease, e.g., a Cas9 nuclease. Various Cas9 nucleases are suitable for use with the gRNAs provided herein to effect genome editing according to aspects of this disclosure, e.g., to create a genomic modification in the EMR2 gene. Typically, the Cas nuclease and the gRNA are provided in a form and under conditions suitable for the formation of a Cas/gRNA complex, that targets a target site on the genome of the cell, e.g., a target site within the EMR2 gene. In some embodiments, a Cas nuclease is used that exhibits a desired PAM specificity to target the Cas/gRNA complex to a desired target domain in the EMR2 gene. Suitable target domains and corresponding gRNA targeting domain sequences are provided herein.

In some embodiments, a Cas/gRNA complex is formed, e.g., in vitro, and a target cell is contacted with the Cas/gRNA complex, e.g., via electroporation of the Cas/gRNA complex into the cell. In some embodiments, the cell is contacted with the Cas protein and gRNA separately, and the Cas/gRNA complex is formed within the cell. In some embodiments, the cell is contacted with a nucleic acid, e.g., a DNA or RNA, encoding the Cas protein, and/or with a nucleic acid encoding the gRNA, or both.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71: 1-9. In some embodiments, a Cas nuclease used in the methods of genome editing provided herein is a Cpf1 nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7™ (Inscripta).

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure. In some embodiments, the Cas nuclease is a variant having reduced PAM sequence specificity. In some embodiments, such a gRNA is referred to as “PAMless” or “near PAMless.” In some embodiments, the Cas nuclease is a SpRY nuclease. See, e.g., Walton et al., Science. 2020 Apr. 17; 368(6488):290-296, which is incorporated by reference herein.

Some features of some exemplary, non-limiting suitable Cas nucleases are described in more detail herein, without wishing to be bound to any particular theory.

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat: anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science, 343(6176): 1247997, 2014) and for S. pyogenes Cas9 with a guide RNA (e.g., a synthetic fusion of crRNA and tracrRNA) (see, e.g., Nishimasu et al., Cell (2014) 156:935-949; and Anders et al., Nature (2014) doi: 10.1038/nature13579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSpCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, SpCas9 and SaCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 (Cas9 from Francisella novicida) recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35(8): 789-792.

In some embodiments, more than one (e.g., 2, 3, or more) Cas molecules are used. In some embodiments, more than one (e.g., 2, 3, or more) Cas9 molecules are used. In some embodiments, at least one of the Cas9 molecules is a Cas9 enzyme. In some embodiments, at least one of the Cas molecules is a Cpf1 enzyme. In some embodiments, at least one of the Cas9 molecule is derived from Streptococcus pyogenes. In some embodiments, at least one of the Cas9 molecule is derived from Streptococcus pyogenes and at least one Cas9 molecule is derived from an organism that is not Streptococcus pyogenes.

In some embodiments, a base editor is used to create a genomic modification resulting in a loss of expression of EMR2, or in expression of a EMR2 variant not targeted by an immunotherapy. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive Cas nuclease is referred to as “dead Cas” or “dCas.” In some embodiments, the catalytically inactive Cas molecule has reduced activity and is, e.g., a nickase (nCas). In some embodiments, the endonuclease comprises a dCas or nCas fused to an adenine base editor (ABE), for example an ABE evolved from the RNA adenine deaminase TadA. In some embodiments, the endonuclease comprises a dCas or nCas fused to a cytosine base editor (CBE), for example a CBE evolved from the cytidine deaminase enzyme (e.g., APOBEC deaminase, pmCDA1, activation-induced cytidine deaminase (AID)).

In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is a Cas9 nickase (nCas9). In some embodiments, the catalytically inactive Cas9 molecule (dCas9) is fused to one or more uracil glycosylase inhibitor (UGI) domains. In some embodiments, the Cas9 molecule comprises a catalytically Cas9 molecule (dCas9) fused to an adenine base editor (ABE), for example an ABE evolved from the RNA adenine deaminase TadA. In some embodiments, the Cas9 molecule comprises a nCas9 fused to an adenine base editor (ABE), for example an ABE evolved from the RNA adenine deaminase TadA. In some embodiments, the Cas9 molecule comprises a dCas9 fused to cytidine deaminase enzyme (e.g., APOBEC deaminase, pmCDA1, activation-induced cytidine deaminase (AID)). In some embodiments, the Cas9 molecule comprises a nCas9 fused to cytidine deaminase enzyme (e.g., APOBEC deaminase, pmCDA1, activation-induced cytidine deaminase (AID)).

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, ABE8, ABE8e, xABE, ABESa, VQR-ABE, VRER-ABE, Sa(KKH)-ABE, CBE, CBE1, CBE2, CBE3, CBE4, and CRISPR-SKIP. Additional examples of base editors can be found, for example, in U.S. Publication No. 2018/0312825A1, U.S. Publication No. 2018/0312828A1, and PCT Publication No. WO 2018/165629A1, which are incorporated by reference herein in their entireties.

Some aspects of this disclosure provide guide RNAs that are suitable to target an RNA-guided nuclease, e.g. as provided herein, to a suitable target site in the genome of a cell in order to effect a modification in the genome of the cell that results in a loss of expression of EMR2, or expression of a variant form of EMR2 that is not recognized by an immunotherapeutic agent targeting EMR2.

The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNA sequence).

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO2014/093694, and PCT Publication No. WO2013/176772, which are herein incorporated by reference in their entireties.

For example, the binding domains of naturally occurring SpCas9 gRNAs typically comprise two RNA molecules, the crRNA (partially) and the tracrRNA. Variants of SpCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” Suitable gRNAs for use with other Cas nucleases, for example, with Cas12a nucleases, typically comprise only a single RNA molecule, as the naturally occurring Cas12a guide RNA comprises a single RNA molecule. A suitable gRNA may thus be unimolecular (having a single RNA molecule), sometimes referred to herein as sgRNAs, or modular (comprising more than one, and typically two, separate RNA molecules).

A gRNA suitable for targeting a target site in the EMR2 gene may comprise a number of domains. In some embodiments, e.g., in some embodiments where a Cas9 nuclease is used, a unimolecular sgRNA, may comprise, from 5′ to 3′:
a targeting domain corresponding to a target site sequence in the EMR2 gene;
a first complementarity domain;
a linking domain;
a second complementarity domain (which is complementary to the first complementarity domain);
a proximal domain; and
optionally, a tail domain.

Each of these domains is now described in more detail.

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. (2019) 6: 6, which is incorporated by reference herein. For additional illustration and description of the mechanism of gRNA targeting an RNA-guided nuclease to a target site, see Fu Y et al, Nat Biotechnol (2014) (doi: 10.1038/nbt.2808) and Sternberg S H et al., Nature (2014) (doi: 10.1038/nature13011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

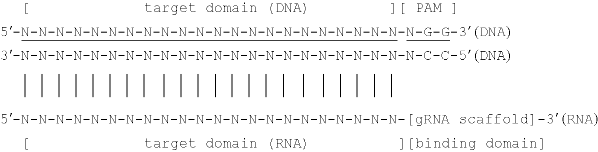

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

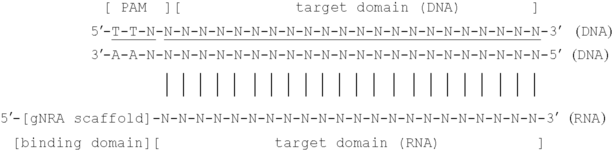

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference.

In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24, 25, 26, or 27 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, 25, 26, or 27 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
   a targeting domain (which corresponds to a target domain in the EMR2 gene); and
   a first complementarity domain; and
a second strand, comprising, e.g., from 5′ to 3′:
   optionally, a 5′ extension domain;
   a second complementarity domain;
   a proximal domain; and
   optionally, a tail domain.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the gRNA may comprise one or more modified nucleotides, e.g., as described in PCT Publication Nos. WO2017/214460, WO2016/089433, and WO2016/164356, which are incorporated by reference their entireties.

The EMR2 targeting gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

The present disclosure provides a number of EMR2 target sites and corresponding gRNAs that are useful for targeting an RNA-guided nuclease to human EMR2. Table 1 below illustrates preferred target domains in the human endogenous EMR2 gene that can be bound by gRNAs described herein. The exemplary target sequences of human EMR2 shown in Table 1, in some embodiments, are for use with a Cas9 nuclease, e.g., SpCas9.

TABLE 1
Exemplary Cas9 target site sequences of human
EMR2 are provided, as are exemplary gRNA
targeting domain sequences useful for targeting
such sites. For each target site, the first
sequence represents the DNA target domain
sequence, the second sequence represents the
reverse complement thereof, and the third
sequence represents an exemplary targeting domain
sequence of a gRNA that can be used to target
the respective target site.

| Guide | |
|---|---|
| Name | Target Domain Sequence |
| EMR2-1 | CGGGCTGGCAACCGATTCCG (SEQ ID NO: 1) |
| | CGGAATCGGTTGCCAGCCCG (SEQ ID NO: 30) |
| | CGGGCUGGCAACCGAUUCCG (SEQ ID NO: 59) |
| EMR2-2 | CCGATTCCGGGGTCCCCCAA (SEQ ID NO: 2) |
| | TTGGGGGACCCCGGAATCGG (SEQ ID NO: 31) |
| | CCGAUUCCGGGGUCCCCCAA (SEQ ID NO: 60) |
| EMR2-3 | CGGAATCCCGAATAACCAAA (SEQ ID NO: 3) |
| | TTTGGTTATTCGGGATTCCG (SEQ ID NO: 32) |
| | CGGAAUCCCGAAUAACCAAA (SEQ ID NO: 61) |
| EMR2-4 | CCCGGAATCGGTTGCCAGCC (SEQ ID NO: 4) |
| | GGCTGGCAACCGATTCCGGG (SEQ ID NO: 33) |
| | CCCGGAAUCGGUUGCCAGCC (SEQ ID NO: 62) |
| EMR2-5 | GTTATTCGGGATTCCGTGTC (SEQ ID NO: 5) |
| | GACACGGAATCCCGAATAAC (SEQ ID NO: 6) |
| | GUUAUUCGGGAUUCCGUGUC (SEQ ID NO: 63) |
| EMR2-6 | GATGCAGCTCGACTGGAATC (SEQ ID NO: 82) |
| | GATTCCAGTCGAGCTGCATC (SEQ ID NO: 106) |
| | GAUGCAGCUCGACUGGAAUC (SEQ ID NO: 64) |
| EMR2-7 | CCAGCGCCTGTAAGATGCTC (SEQ ID NO: 7) |
| | GAGCATCTTACAGGCGCTGG (SEQ ID NO: 36) |
| | CCAGCGCCUGUAAGAUGCUC (SEQ ID NO: 65) |
| EMR2-8 | CCAGAGCATCTTACAGGCGC (SEQ ID NO: 8) |
| | GCGCCTGTAAGATGCTCTGG (SEQ ID NO: 37) |
| | CCAGAGCAUCUUACAGGCGC (SEQ ID NO: 66) |
| EMR2-9 | CCTTACCTGGATGGTGTTAT (SEQ ID NO: 9) |
| | ATAACACCATCCAGGTAAGG (SEQ ID NO: 38) |
| | CCUUACCUGGAUGGUGUUAU (SEQ ID NO: 67) |
| EMR2-10 | CCAATAACACCATCCAGGTA (SEQ ID NO: 10) |
| | TACCTGGATGGTGTTATTGG (SEQ ID NO: 39) |
| | CCAAUAACACCAUCCAGGUA (SEQ ID NO: 68) |
| EMR2-11 | CTTGGCCAATAACACCATCC (SEQ ID NO: 11) |
| | GGATGGTGTTATTGGCCAAG (SEQ ID NO: 40) |
| | CUUGGCCAAUAACACCAUCC (SEQ ID NO: 69) |
| EMR2-12 | CAGAGACTACAAGCCAGGCT (SEQ ID NO: 12) |
| | AGCCTGGCTTGTAGTCTCTG (SEQ ID NO: 41) |
| | CAGAGACUACAAGCCAGGCU (SEQ ID NO: 70) |
| EMR2-13 | CTGGGCAGAGACTACAAGCC (SEQ ID NO: 13) |
| | GGCTTGTAGTCTCTGCCCAG (SEQ ID NO: 42) |
| | CUGGGCAGAGACUACAAGCC (SEQ ID NO: 71) |
| EMR2-14 | TACACGTGCCAGTGCCTGCC (SEQ ID NO: 14) |
| | GGCAGGCACTGGCACGTGTA (SEQ ID NO: 43) |
| | UACACGUGCCAGUGCCUGCC (SEQ ID NO: 72) |
| EMR2-15 | TGTACCGTCACAAGTCTCCA (SEQ ID NO: 15) |
| | TGGAGACTTGTGACGGTACA (SEQ ID NO: 44) |
| | UGUACCGUCACAAGUCUCCA (SEQ ID NO: 73) |
| EMR2-16 | TACCGTCACAAGTCTCCATG (SEQ ID NO: 16) |
| | CATGGAGACTTGTGACGGTA (SEQ ID NO: 45) |
| | UACCGUCACAAGUCUCCAUG (SEQ ID NO: 74) |
| EMR2-17 | CGCGTCTTTCTCGTCTTTCT (SEQ ID NO: 17) |
| | AGAAAGACGAGAAAGACGCG (SEQ ID NO: 46) |
| | CGCGUCUUUCUCGUCUUUCU (SEQ ID NO: 75) |
| EMR2-18 | CGGCAGCTCAGCTGGAACCA (SEQ ID NO: 18) |
| | TGGTTCCAGCTGAGCTGCCG (SEQ ID NO: 47) |
| | CGGCAGCUCAGCUGGAACCA (SEQ ID NO: 76) |

The present disclosure provides exemplary EMR2 targeting gRNAs that are useful for targeting an RNA-guided nuclease to human EMR2. Table 2 below illustrates preferred targeting domains for use in gRNAs targeting Cas9 nucleases to human endogenous EMR2 gene. The exemplary target sequences of human EMR2 shown in Table 2, in some embodiments, are for use with a Cas9 nuclease, e.g., SpCas9.

TABLE 2
Exemplary Cas9 targeting domain sequences of
gRNAs targeted to human EMR2 are provided.

| Guide Name | Target Domain Sequence |
|---|---|
| EMR2-1 | CGGGCUGGCAACCGAUUCCG (SEQ ID NO: 59) |
| EMR2-2 | CCGAUUCCGGGGUCCCCCAA (SEQ ID NO: 60) |
| EMR2-3 | CGGAAUCCCGAAUAACCAAA (SEQ ID NO: 61) |
| EMR2-4 | CCCGGAAUCGGUUGCCAGCC (SEQ ID NO: 62) |
| EMR2-5 | GUUAUUCGGGAUUCCGUGUC (SEQ ID NO: 63) |
| EMR2-6 | GAUGCAGCUCGACUGGAAUC (SEQ ID NO: 64) |
| EMR2-7 | CCAGCGCCUGUAAGAUGCUC (SEQ ID NO: 65) |
| EMR2-8 | CCAGAGCAUCUUACAGGCGC (SEQ ID NO: 66) |
| EMR2-9 | CCUUACCUGGAUGGUGUUAU (SEQ ID NO: 67) |
| EMR2-10 | CCAAUAACACCAUCCAGGUA (SEQ ID NO: 68) |
| EMR2-11 | CUUGGCCAAUAACACCAUCC (SEQ ID NO: 69) |
| EMR2-12 | CAGAGACUACAAGCCAGGCU (SEQ ID NO: 70) |
| EMR2-13 | CUGGGCAGAGACUACAAGCC (SEQ ID NO: 71) |
| EMR2-14 | UACACGUGCCAGUGCCUGCC (SEQ ID NO: 72) |
| EMR2-15 | UGUACCGUCACAAGUCUCCA (SEQ ID NO: 73) |
| EMR2-16 | UACCGUCACAAGUCUCCAUG (SEQ ID NO: 74) |
| EMR2-17 | CGCGUCUUUCUCGUCUUUCU (SEQ ID NO: 75) |
| EMR2-18 | CGGCAGCUCAGCUGGAACCA (SEQ ID NO: 76) |

Exemplary base editing target site sequences of human EMR2 are provided, as are exemplary gRNA targeting domain sequences useful for targeting such sites. For each target site, the first sequence represents the DNA target domain sequence, the second sequence represents the reverse complement thereof, and the third sequence represents an exemplary targeting domain sequence of a gRNA that can be used to target the respective target site. The nucleotide residue shown in boldface and with underline indicates the nucleotide that is predicted to be mutated by the base editor.

The exemplary target sequences of human EMR2 shown in Table 3, in some embodiments, are for use, for example with cytosine base editors (CBEs).

TABLE 3
Exemplary CBE targeting domain sequences of gRNAs targeted to human EMR2
are provided.

| | | | Amino Acid |
|---|---|---|---|
| Guide | | | Change/Splice |
| Name | Target Domain Sequence | Exon | Site |
| EMR2-19 | CTATCAGTGCCGCTGCCGCC (SEQ ID NO: 77) | 7 | CAG→TAG |
| | GGCGGCAGCGGCACTGATAG (SEQ ID NO: 101) | | |
| | CUAUCAGUGCCGCUGCCGCC (SEQ ID NO: 125) | | |
| EMR2-20 | TATCAGTGCCGCTGCCGCCC (SEQ ID NO: 78) | 7 | CAG→TAG |
| | GGGCGGCAGCGGCACTGATA (SEQ ID NO: 102) | | |
| | UAUCAGUGCCGCUGCCGCCC (SEQ ID NO: 126) | | |
| EMR2-21 | GTTGCCAGCCCGGGCGGCAG (SEQ ID NO: 79) | 7 | TGG→TAA |
| | CTGCCGCCCGGGCTGGCAAC (SEQ ID NO: 103) | | |
| | GUUGCCAGCCCGGGCGGCAG (SEQ ID NO: 127) | | |
| EMR2-22 | GCTTCCAGCCTGGGCGGCAG (SEQ ID NO: 80) | 8 | TGG→TAA |
| | CTGCCGCCCAGGCTGGAAGC (SEQ ID NO: 104) | | |
| | GCUUCCAGCCUGGGCGGCAG (SEQ ID NO: 128) | | |
| EMR2-23 | CCGCTTACAGCAGCACTGTG (SEQ ID NO: 81) | 11 | CAG→TAG |
| | CACAGTGCTGCTGTAAGCGG (SEQ ID NO: 105) | | |
| | CCGCUUACAGCAGCACUGUG (SEQ ID NO: 129) | | |
| EMR2-24 | GATGCAGCTCGACTGGAATC (SEQ ID NO: 82) | 12 | CAG→TAG |
| | GATTCCAGTCGAGCTGCATC (SEQ ID NO: 106) | | |
| | GAUGCAGCUCGACUGGAAUC (SEQ ID NO: 64) | | |
| EMR2-25 | TGGAATCAGGCACAGAAATC (SEQ ID NO: 83) | 12 | CAG→TAG |
| | GATTTCTGTGCCTGATTCCA (SEQ ID NO: 107) | | |
| | UGGAAUCAGGCACAGAAAUC (SEQ ID NO: 131) | | |
| EMR2-26 | AGGTGCAGAAGCAAGTAGAC (SEQ ID NO: 84) | 12 | CAG→TAG |
| | GTCTACTTGCTTCTGCACCT (SEQ ID NO: 108) | | |
| | AGGUGCAGAAGCAAGUAGAC (SEQ ID NO: 132) | | |
| EMR2-27 | GCACAGAAATCTGGTGACCC (SEQ ID NO: 85) | 12 | CAG→TAG |
| | GGGTCACCAGATTTCTGTGC (SEQ ID NO: 109) | | |
| | GCACAGAAAUCUGGUGACCC (SEQ ID NO: 133) | | |
| EMR2-28 | GACACACCAGGGCTTGCTGC (SEQ ID NO: 86) | 13 | CAG→TAG |
| | GCAGCAAGCCCTGGTGTGTC (SEQ ID NO: 110) | | |
| | GACACACCAGGGCUUGCUGC (SEQ ID NO: 134) | | |
| EMR2-29 | CACCAGGGCTTGCTGCAGGA (SEQ ID NO: 87) | 13 | CAG→TAG |
| | TCCTGCAGCAAGCCCTGGTG (SEQ ID NO: 111) | | |
| | CACCAGGGCUUGCUGCAGGA (SEQ ID NO: 135) | | |
| EMR2-30 | GATGTGCAGGTGAGACCCTT (SEQ ID NO: 88) | 14 | CAG→TAG |
| | AAGGGTCTCACCTGCACATC (SEQ ID NO: 112) | | |
| | GAUGUGCAGGUGAGACCCUU (SEQ ID NO: 136) | | |
| EMR2-31 | AATTGATCAAACCGGACACA (SEQ ID NO: 89) | 15 | CAA→TAA |
| | TGTGTCCGGTTTGATCAATT (SEQ ID NO: 113) | | |
| | AAUUGAUCAAACCGGACACA (SEQ ID NO: 137) | | |
| EMR2-32 | GCATCCAGGTCAAGGTGGCC (SEQ ID NO: 90) | 16 | TGG→TAA |
| | GGCCACCTTGACCTGGATGC (SEQ ID NO: 114) | | |
| | GCAUCCAGGUCAAGGUGGCC (SEQ ID NO: 138) | | |
| EMR2-33 | CACATCTGAGGACAGGAAGA (SEQ ID NO: 91) | 8 | Splice acceptor |
| | TCTTCCTGTCCTCAGATGTG (SEQ ID NO: 115) | | disruption |
| | CACAUCUGAGGACAGGAAGA (SEQ ID NO: 139) | | |
| EMR2-34 | ACATCTGCAAGAGGAAGGAG (SEQ ID NO: 92) | 6 | Splice acceptor |
| | CTCCTTCCTCTTGCAGATGT (SEQ ID NO: 116) | | disruption |
| | ACAUCUGCAAGAGGAAGGAG (SEQ ID NO: 140) | | |
| EMR2-35 | ACATCTGAGGACAGGAAGAA (SEQ ID NO: 93) | 8 | Splice acceptor |
| | TTCTTCCTGTCCTCAGATGT (SEQ ID NO: 117) | | disruption |
| | ACAUCUGAGGACAGGAAGAA (SEQ ID NO: 141) | | |
| EMR2-36 | TGTCTGGAACACAACAGGAC (SEQ ID NO: 94) | 5 | Splice acceptor |
| | GTCCTGTTGTGTTCCAGACA (SEQ ID NO: 118) | | disruption |
| | UGUCUGGAACACAACAGGAC (SEQ ID NO: 142) | | |
| EMR2-37 | CATCTGAGGACAGGAAGAAG (SEQ ID NO: 95) | 8 | Splice acceptor |
| | CTTCTTCCTGTCCTCAGATG (SEQ ID NO: 119) | | disruption |
| | CAUCUGAGGACAGGAAGAAG (SEQ ID NO: 143) | | |
| EMR2-38 | ATCTGAGGACAGGAAGAAGG (SEQ ID NO: 96) | 8 | Splice acceptor |
| | CCTTCTTCCTGTCCTCAGAT (SEQ ID NO: 120) | | disruption |
| | AUCUGAGGACAGGAAGAAGG (SEQ ID NO: 144) | | |
| EMR2-39 | GGGCCTGCAGGGCGAGGCCA (SEQ ID NO: 97) | 13 | Splice acceptor |
| | TGGCCTCGCCCTGCAGGCCC (SEQ ID NO: 121) | | disruption |
| | GGGCCUGCAGGGCGAGGCCA (SEQ ID NO: 145) | | |
| EMR2-40 | TAAGATGCTCTGGAGGGATG (SEQ ID NO: 98) | 11 | Splice acceptor |
| | CATCCCTCCAGAGCATCTTA (SEQ ID NO: 122) | | disruption |
| | UAAGAUGCUCUGGAGGGAUG (SEQ ID NO: 146) | | |
| EMR2-41 | TGACTGCAGGAACAGAACAC (SEQ ID NO: 99) | 14 | Splice acceptor |
| | GTGTTCTGTTCCTGCAGTCA (SEQ ID NO: 123) | | disruption |
| | UGACUGCAGGAACAGAACAC (SEQ ID NO: 147) | | |
| EMR2-42 | ACATCTGAGGACAAAGCCAG (SEQ ID NO: 100) | 7 | Splice acceptor |
| | CTGGCTTTGTCCTCAGATGT (SEQ ID NO: 124) | | disruption |
| | ACAUCUGAGGACAAAGCCAG (SEQ ID NO: 148) | | |
| EMR2-55 | ACAGGCGCTGGATGAGCTGC (SEQ ID NO: 49) | | Missense variant |
| | GCAGCTCATCCAGCGCCTGT (SEQ ID NO: 52) | | |
| | ACAGGCGCUGGAUGAGCUGC (SEQ ID NO: 165) | | |
| EMR2-60 | TGTAGTCTCTGCCCAGGTCC (SEQ ID NO: 177) | | Missense variant |
| | GGACCTGGGCAGAGACTACA (SEQ ID NO: 178) | | |
| | UGUAGUCUCUGCCCAGGUCC (SEQ ID NO: 179) | | |
| EMR2-8 | CCAGAGCATCTTACAGGCGC (SEQ ID NO: 8) | | Splice region |
| | GCGCCTGTAAGATGCTCTGG (SEQ ID NO: 37) | | variant |
| | CCAGAGCAUCUUACAGGCGC (SEQ ID NO: 66) | | |
| EMR2-7 | CCAGCGCCTGTAAGATGCTC (SEQ ID NO: 7) | | Synonymous |
| | GAGCATCTTACAGGCGCTGG (SEQ ID NO: 36) | | variant/missense |
| | CCAGCGCCUGUAAGAUGCUC (SEQ ID NO: 65) | | variant |
| EMR2-45 | GCGCCTGTAAGATGCTCTGG (SEQ ID NO: 37) | | Missense variant |
| | CCAGAGCATCTTACAGGCGC (SEQ ID NO: 8) | | |
| | GCGCCUGUAAGAUGCUCUGG (SEQ ID NO: 180) | | |
| EMR2-91 | GGCGCTGGATGAGCTGCTGG (SEQ ID NO: 181) | | |
| | CCAGCAGCTCATCCAGCGCC (SEQ ID NO: 182) | | |
| | GGCGCUGGAUGAGCUGCUGG (SEQ ID NO: 183) | | |
| EMR2-61 | CGCCTGTAAGATGCTCTGGA (SEQ ID NO: 184) | | |
| | TCCAGAGCATCTTACAGGCG (SEQ ID NO: 185) | | |
| | CGCCUGUAAGAUGCUCUGGA (SEQ ID NO: 186) | | |
| EMR2-40 | TAAGATGCTCTGGAGGGATG (SEQ ID NO: 98) | | |
| | CATCCCTCCAGAGCATCTTA (SEQ ID NO: 122) | | |
| | UAAGAUGCUCUGGAGGGAUG (SEQ ID NO: 146) | | |
| EMR2-58 | AACACCATCCAGGTAAGGAC (SEQ ID NO: 187) | | |
| | GTCCTTACCTGGATGGTGTT (SEQ ID NO: 188) | | |
| | AACACCAUCCAGGUAAGGAC (SEQ ID NO: 189) | | |
| EMR2-9 | CCTTACCTGGATGGTGTTAT (SEQ ID NO: 9) | | |
| | ATAACACCATCCAGGTAAGG (SEQ ID NO: 38) | | |
| | CCUUACCUGGAUGGUGUUAU (SEQ ID NO: 67) | | |
| EMR2-11 | CTTGGCCAATAACACCATCC (SEQ ID NO: 11) | | |
| | GGATGGTGTTATTGGCCAAG (SEQ ID NO: 40) | | |
| | CUUGGCCAAUAACACCAUCC (SEQ ID NO: 69) | | |
| EMR2-72 | GGCtTGGCCAATAACACCAT (SEQ ID NO: 211) | | |
| | ATGGTGTTATTGGCCAaGCC (SEQ ID NO: 212) | | |
| | GGCuUGGCCAAUAACACCAU (SEQ ID NO: 213) | | |
| EMR2-73 | TGAGCTGcTGGAGGCCCCTG (SEQ ID NO: 214) | | |
| | CAGGGGCCTCCAgCAGCTCA (SEQ ID NO: 215) | | |
| | UGAGCUGcUGGAGGCCCCUG (SEQ ID NO: 216) | | |
| EMR2-74 | CTGTGTGGCCAGTCACCTGC (SEQ ID NO: 217) | | |
| | GCAGGTGACTGGCCACACAG (SEQ ID NO: 218) | | |
| | CUGUGUGGCCAGUCACCUGC (SEQ ID NO: 219) | | |
| EMR2-75 | AGTCACCTGCTGGATGGCCT (SEQ ID NO: 220) | | |
| | AGGCCATCCAGCAGGTGACT (SEQ ID NO: 221) | | |
| | AGUCACCUGCUGGAUGGCCU (SEQ ID NO: 222) | | |
| EMR2-76 | AGAGGATgTCCTCAGAGGCC (SEQ ID NO: 223) | | |
| | GGCCTCTGAGGAcATCCTCT (SEQ ID NO: 224) | | |
| | AGAGGAUgUCCUCAGAGGCC (SEQ ID NO: 225) | | |
| EMR2-77 | CAATAACACCATCCAGGTAA (SEQ ID NO: 226) | | |
| | TTACCTGGATGGTGTTATTG (SEQ ID NO: 227) | | |
| | CAAUAACACCAUCCAGGUAA (SEQ ID NO: 228) | | |
| EMR2-78 | CAGAGCATCTTACAGGCGCT (SEQ ID NO: 229) | | |
| | AGCGCCTGTAAGATGCTCTG (SEQ ID NO: 230) | | |
| | CAGAGCAUCUUACAGGCGCU (SEQ ID NO: 231) | | |
| EMR2-79 | CAGgCGCTGGATGAGCTGCT (SEQ ID NO: 232) | | |
| | AGCAGCTCATCCAGCGcCTG (SEQ ID NO: 233) | | |
| | CAGgCGCUGGAUGAGCUGCU (SEQ ID NO: 234) | | |
| EMR2-80 | TGTgTGGCCAGTCACCTGCT (SEQ ID NO: 235) | | |
| | AGCAGGTGACTGGCCAcACA (SEQ ID NO: 236) | | |
| | UGUgUGGCCAGUCACCUGCU (SEQ ID NO: 237) | | |
| EMR2-81 | AGCaTCTTACAGGCGCTGGA (SEQ ID NO: 238) | | |
| | TCCAGCGCCTGTAAGAtGCT (SEQ ID NO: 239) | | |
| | AGCaUCUUACAGGCGCUGGA (SEQ ID NO: 240) | | |

TABLE 4
Exemplary gRNAs targeted to human EMR2 are provided
for editing with a CBE.

| | | | Base | | | Amino |
|---|---|---|---|---|---|---|
| | | | editing | Amino | | Acid |
| Guide | Sequence | PAM | sequence | Acids | Edit | Position |
| EMR2-91 | GGCGCTGGATGAGCTGCTGG | AGG | tGcTGG | A->V | Missense | 308 |
| | (SEQ ID NO: 181) | | tGtTGG | AL->VL | variant | |
| EMR2-55 | ACAGGCGCTGGATGAGCTGC | TGG | AGGtGc | A->V | Missense | 307 |
| | (SEQ ID NO: 49) | | AGGtGt | AL->VL | variant | |
| EMR2-8 | CCAGAGCATCTTACAGGCGC | TGG | AGAGtA | S | Splice | 303 |
| | (SEQ ID NO: 8) | | | | region | |
| | | | | | variant | |
| EMR2-7 | CCAGCGCCTGTAAGATGCTC | TGG | AGtGcc | AA->T | Synonymous | 307 |
| | (SEQ ID NO: 7) | | AgtGtc | QA->QT | variant/ | |
| | | | AgtGtt | | missense | |
| | | | | | variant | |
| EMR2-8 | GCGCCTGTAAGATGCTCTGG | AGG | GtcTGT | A->T | Missense | 306, |
| | (SEQ ID NO: 37) | | GttTGT | QA->QT | variant | 307 |
| EMR2-61 | CGCCTGTAAGATGCTCTGGA | GGG | CtTGTA | QQA->QT | Synonymous | 306 |
| | (SEQ ID NO: 184) | | ttTGTA | | variant/ | |
| | | | | | missense | |
| | | | | | variant | |
| EMR2-40 | TAAGATGCTCTGGAGGGATG | TGG | AGATGt | S->N | Splice | 303 |
| | (SEQ ID NO: 98) | | | | region | |
| | | | | | variant | |
| EMR2-58 | AACACCATCCAGGTAAGGAC | AGG | TAccAT | NNT->NI | Synonymous | 300 |
| | (SEQ ID NO: 187) | | tAtcAT | NT->NI | variant/ | |
| | | | tAttAT | | missense | |
| | | | | | variant | |
| EMR2-9 | CCTTACCTGGATGGTGTTAT | TGG | TTAtcT | — | Splice | 302 |
| | (SEQ ID NO: 9) | | TTAttT | | region | |
| | | | | | variant | |
| EMR2-11 | CTTGGCCAATAACACCATCC | AGG | TGGtcA | A->V | Missense | 297 |
| | (SEQ ID NO: 11) | | TGGttA | | variant | |
| EMR2-60 | TGTAGTCTCTGCCCAGGTCC | TGG | TAGTtT | D->N | Missense | 291 |
| | (SEQ ID NO: 177) | | | | variant | |

The exemplary target sequences of human EMR2 shown in Table 5, in some embodiments, are for use, for example with adenine base editors (ABEs). For each target site, the first sequence represents the DNA target domain sequence, the second sequence represents the reverse complement thereof, and the third sequence represents an exemplary targeting domain sequence of a gRNA that can be used to target the respective target site.

TABLE 5
Exemplary ABE targeting domain sequences of gRNAs
targeted to human EMR2 are provided.

| | | | Amino Acid |
|---|---|---|---|
| Guide | | | Change/Splice |
| Name | Target Domain Sequence | Exon | Site |
| EMR2-43 | CCTCTACCTGTGCAGAGCTT (SEQ ID NO: 149) | 6 | Splice donor |
| | AAGCTCTGCACAGGTAGAGG (SEQ ID NO: 155) | | disruption |
| | CCUCUACCUGUGCAGAGCUU (SEQ ID NO: 161) | | |
| EMR2-44 | CTCTACCTGIGCAGAGCTTC (SEQ ID NO: 150) | 6 | Splice donor |
| | GAAGCTCTGCACAGGTAGAG (SEQ ID NO: 156) | | disruption |
| | CUCUACCUGUGCAGAGCUUC (SEQ ID NO: 162) | | |
| EMR2-9 | CCTTACCTGGATGGTGTTAT (SEQ ID NO: 9) | 10 | Splice donor |
| | ATAACACCATCCAGGTAAGG (SEQ ID NO: 38) | | disruption |
| | CCUUACCUGGAUGGUGUUAU (SEQ ID NO: 67) | | |
| EMR2-46 | AGACTCACCCCTGGAGTCCT (SEQ ID NO: 152) | 3 | Splice donor |
| | AGGACTCCAGGGGTGAGTCT (SEQ ID NO: 158) | | disruption |
| | AGACUCACCCCUGGAGUCCU (SEQ ID NO: 164) | | |
| EMR2-15 | TGTACCGTCACAAGTCTCCA (SEQ ID NO: 15) | 4 | Splice donor |
| | TGGAGACTTGTGACGGTACA (SEQ ID NO: 44) | | disruption |
| | UGUACCGUCACAAGUCUCCA (SEQ ID NO: 73) | | |
| EMR2-48 | CTCACCTGGCTGTGGACTCC (SEQ ID NO: 154) | 9 | Splice donor |
| | GGAGTCCACAGCCAGGTGAG (SEQ ID NO: 160) | | disruption |
| | CUCACCUGGCUGUGGACUCC (SEQ ID NO: 166) | | |
| EMR2-49 | GGCAGGGTCTCCAGGTCCCC (SEQ ID NO: 241) | | Missense variant |
| | GGGGACCTGGAGACCCTGCC (SEQ ID NO: 242) | | |
| | GGCAGGGUCUCCAGGUCCCC (SEQ ID NO: 243) | | |
| EMR2-50 | GCAGGGTCTCCAGGTCCCCA (SEQ ID NO: 244) | | Missense variant |
| | TGGGGACCTGGAGACCCTGC (SEQ ID NO: 245) | | |
| | GCAGGGUCUCCAGGUCCCCA (SEQ ID NO: 246) | | |
| EMR2-51 | GCTGGAGGCCCCTGGGGACC (SEQ ID NO: 247) | | Missense variant |
| | GGTCCCCAGGGGCCTCCAGC (SEQ ID NO: 248) | | |
| | GCUGGAGGCCCCUGGGGACC (SEQ ID NO: 249) | | |
| EMR2-52 | TGAGCTGCTGGAGGCCCCTG (SEQ ID NO: 214) | | Missense variant |
| | CAGGGGCCTCCAGCAGCTCA (SEQ ID NO: 215) | | |
| | UGAGCUGCUGGAGGCCCCUG (SEQ ID NO: 216) | | |
| EMR2-53 | ATGAGCTGCTGGAGGCCCCT (SEQ ID NO: 250) | | Missense variant |
| | AGGGGCCTCCAGCAGCTCAT (SEQ ID NO: 251) | | |
| | AUGAGCUGCUGGAGGCCCCU (SEQ ID NO: 252) | | |
| EMR2-54 | GATGAGCTGCTGGAGGCCCC (SEQ ID NO: 253) | | Missense variant |
| | GGGGCCTCCAGCAGCTCATC (SEQ ID NO: 254) | | |
| | GAUGAGCUGCUGGAGGCCCC (SEQ ID NO: 255) | | |
| EMR2-55 | ACAGGCGCTGGATGAGCTGC (SEQ ID NO: 49) | | Missense variant |
| | GCAGCTCATCCAGCGCCTGT (SEQ ID NO: 52) | | |
| | ACAGGCGCUGGAUGAGCUGC (SEQ ID NO: 165) | | |
| EMR2-7 | CCAGCGCCTGTAAGATGCTC (SEQ ID NO: 7) | | Missense variant |
| | GAGCATCTTACAGGCGCTGG (SEQ ID NO: 36) | | |
| | CCAGCGCCUGUAAGAUGCUC (SEQ ID NO: 65) | | |
| EMR2-40 | TAAGATGCTCTGGAGGGATG (SEQ ID NO: 98) | | Synonymous |
| | CATCCCTCCAGAGCATCTTA (SEQ ID NO: 122) | | variant |
| | UAAGAUGCUCUGGAGGGAUG (SEQ ID NO: 146) | | Missense variant |
| EMR2-56 | CCAGGTAAGGACAGGACCCA (SEQ ID NO: 249) | | Splice region |
| | TGGGTCCTGTCCTTACCTGG (SEQ ID NO: 250) | | variant |
| | CCAGGUAAGGACAGGACCCA (SEQ ID NO: 251) | | |
| EMR2-57 | TCCAGGTAAGGACAGGACCC (SEQ ID NO: 252) | | Splice region |
| | GGGTCCTGTCCTTACCTGGA (SEQ ID NO: 253) | | variant |
| | UCCAGGUAAGGACAGGACCC (SEQ ID NO: 254) | | |
| EMR2-58 | AACACCATCCAGGTAAGGAC (SEQ ID NO: 187) | | Missense variant |
| | GTCCTTACCTGGATGGTGTT (SEQ ID NO: 188) | | |
| | AACACCAUCCAGGUAAGGAC (SEQ ID NO: 189) | | |
| EMR2-10 | CCAATAACACCATCCAGGTA (SEQ ID NO: 10) | | Missense variant |
| | TACCTGGATGGTGTTATTGG (SEQ ID NO: 39) | | |
| | CCAAUAACACCAUCCAGGUA (SEQ ID NO: 68) | | |
| EMR2-11 | CTTGGCCAATAACACCATCC (SEQ ID NO: 11) | | |
| | GGATGGTGTTATTGGCCAAG (SEQ ID NO: 40) | | Missense variant |
| | CUUGGCCAAUAACACCAUCC (SEQ ID NO: 69) | | |
| EMR2-12 | CAGAGACTACAAGCCAGGCT (SEQ ID NO: 12) | 10 | Synonymous |
| | AGCCTGGCTTGTAGTCTCTG (SEQ ID NO: 41) | | variant/Missense |
| | CAGAGACUACAAGCCAGGCU (SEQ ID NO: 70) | | variant |
| EMR2-59 | CTGGGCAGAGACTACAAGCC (SEQ ID NO: 13) | | Missense variant |
| | GGCTTGTAGTCTCTGCCCAG (SEQ ID NO: 42) | | |
| | CUGGGCAGAGACUACAAGCC (SEQ ID NO: 71) | | |
| EMR2-60 | TGTAGTCTCTGCCCAGGTCC (SEQ ID NO: 177) | | Missense variant |
| | GGACCTGGGCAGAGACTACA (SEQ ID NO: 178) | | |
| | UGUAGUCUCUGCCCAGGUCC (SEQ ID NO: 179) | | |
| EMR2-61 | CGCCTGTAAGATGCTCTGGA (SEQ ID NO: 184) | | Missense variant |
| | TCCAGAGCATCTTACAGGCG (SEQ ID NO: 185) | | |
| | CGCCUGUAAGAUGCUCUGGA (SEQ ID NO: 186) | | |
| EMR2-62 | AGCACTCACACGGTGGGAGA (SEQ ID NO: 130) | | |
| | TCTCCCACCGTGTGAGTGCT (SEQ ID NO: 151) | | |
| | AGCACUCACACGGUGGGAGA (SEQ ID NO: 255) | | |
| EMR2-63 | ATCTTACCTTGTGTTCCGGA (SEQ ID NO: 153) | | |
| | TCCGGAACACAAGGTAAGAT (SEQ ID NO: 157) | | |
| | AUCUUACCUUGUGUUCCGGA (SEQ ID NO: 256) | | |
| EMR2-64 | GTGGTACCTGCTGGCTGAGG (SEQ ID NO: 159) | | |
| | CCTCAGCCAGCAGGTACCAC (SEQ ID NO: 163) | | |
| | GUGGUACCUGCUGGCUGAGG (SEQ ID NO: 257) | | |
| EMR2-72 | GGCtTGGCCAATAACACCAT (SEQ ID NO: 211) | | |
| | ATGGTGTTATTGGCCAaGCC (SEQ ID NO: 212) | | |
| | GGCuUGGCCAAUAACACCAU (SEQ ID NO: 213) | | |
| EMR2-8 | CCAGAGCATCTTACAGGCGC (SEQ ID NO: 8) | | |
| | GCGCCTGTAAGATGCTCTGG (SEQ ID NO: 37) | | |
| | CCAGAGCAUCUUACAGGCGC (SEQ ID NO: 66) | | |
| EMR2-81 | AGCaTCTTACAGGCGCTGGA (SEQ ID NO: 238) | | |
| | TCCAGCGCCTGTAAGAtGCT (SEQ ID NO: 239) | | |
| | AGCaUCUUACAGGCGCUGGA (SEQ ID NO: 240) | | |
| EMR2-74 | CTGTGTGGCCAGTCACCTGC (SEQ ID NO: 217) | | |
| | GCAGGTGACTGGCCACACAG (SEQ ID NO: 218) | | |
| | CUGUGUGGCCAGUCACCUGC (SEQ ID NO: 219) | | |
| EMR2-75 | AGTCACCTGCTGGATGGCCT (SEQ ID NO: 220) | | |
| | AGGCCATCCAGCAGGTGACT (SEQ ID NO: 221) | | |
| | AGUCACCUGCUGGAUGGCCU (SEQ ID NO: 222) | | |
| EMR2-76 | AGAGGATgTCCTCAGAGGCC (SEQ ID NO: 223) | | |
| | GGCCTCTGAGGACATCCTCT (SEQ ID NO: 224) | | |
| | AGAGGAUgUCCUCAGAGGCC (SEQ ID NO: 225) | | |
| EMR2-77 | CAATAACACCATCCAGGTAA (SEQ ID NO: 226) | | |
| | TTACCTGGATGGTGTTATTG (SEQ ID NO: 227) | | |
| | CAAUAACACCAUCCAGGUAA (SEQ ID NO: 228) | | |
| EMR2-78 | CAGAGCATCTTACAGGCGCT (SEQ ID NO: 229) | | |
| | AGCGCCTGTAAGATGCTCTG (SEQ ID NO: 230) | | |
| | CAGAGCAUCUUACAGGCGCU (SEQ ID NO: 231) | | |
| EMR2-79 | CAGgCGCTGGATGAGCTGCT (SEQ ID NO: 232) | | |
| | AGCAGCTCATCCAGCGCCTG (SEQ ID NO: 233) | | |
| | CAGgCGCUGGAUGAGCUGCU (SEQ ID NO: 234) | | |
| EMR2-80 | TGTgTGGCCAGTCACCTGCT (SEQ ID NO: 235) | | |
| | AGCAGGTGACTGGCCACACA (SEQ ID NO: 236) | | |
| | UGUgUGGCCAGUCACCUGCU (SEQ ID NO: 237) | | |

TABLE 6
Exemplary gRNAs targeted to human EMR2 are provided for editing with an ABE.

| | | | Base | | | Amino |
|---|---|---|---|---|---|---|
| | | | editing | Amino | | Acid |
| Guide | Sequence | PAM | sequence | Acids | Edit | Position |
| EMR2-49 | GGCAGGGTCTCCAGGTCCCC (SEQ ID NO: 258) | AGG | CgGGGT | L->P | Missense | 321 |
| | | | | | variant | |
| EMR2-50 | GCAGGGTCTCCAGGTCCCCA (SEQ ID NO: 259) | GGG | gGGGTC | L->P | Missense | 321 |
| | | | | | variant | |
| EMR2-51 | GCTGGAGGCCCCTGGGGACC (SEQ ID NO: 260) | TGG | TGGgGG | E->G | Missense | 313 |
| | | | | | variant | |
| EMR2-52 | TGAGCTGCTGGAGGCCCCTG (SEQ ID NO: 214) | GGG | gGCTGC | E->G | Missense | 310 |
| | | | | | variant | |
| EMR2-53 | ATGAGCTGCTGGAGGCCCCT (SEQ ID NO: 261) | GGG | GgGCTG | E->G | Missense | 310 |
| | | | | | variant | |
| EMR2-54 | GATGAGCTGCTGGAGGCCCC (SEQ ID NO: 262) | TGG | TGgGCT | E->G | Missense | 310 |
| | | | | | variant | |
| EMR2-55 | ACAGGCGCTGGATGAGCTGC (SEQ ID NO: 49) | TGG | gGGCGC | Q->R | Missense | 306 |
| | | | | | variant | |
| EMR2-7 | CCAGCGCCTGTAAGATGCTC (SEQ ID NO: 7) | TGG | gGCGCC | L->P | Missense | 308 |
| | | | | | variant | |
| EMR2-61 | CGCCTGTAAGATGCTCTGGA (SEQ ID NO: 184) | GGG | CCTGTg | L->S | Missense | 305 |
| | | | | | variant | |
| EMR2-40 | TAAGATGCTCTGGAGGGATG (SEQ ID NO: 98) | TGG | gGaTGC | L IL->TL | Synonymous | 304 |
| | | | gGgTGC | | variant/ | |
| | | | | | missense | |
| | | | | | variant | |
| EMR2-56 | CCAGGTAAGGACAGGACCCA (SEQ ID NO: 249) | GGG | GGGTaa | Q->R - | Splice | 302 |
| | | | gGGTga | | region | |
| | | | gGGTgg | | variant | |
| EMR2-57 | TCCAGGTAAGGACAGGACCC (SEQ ID NO: 263) | AGG | CgGGTa | Q->R - | Splice region | 302 |
| | | | CgGGTg | | variant | |
| EMR2-58 | AACACCATCCAGGTAAGGAC (SEQ ID NO: 264) | AGG | CgCCaT | T->A | Missense | 300, |
| | | | CgCCgT | TI->AV | variant | 301 |
| EMR2-10 | CCAATAACACCATCCAGGTA (SEQ ID NO: 10) | AGG | agTaaC | N->S | Missense | 298, |
| | | | ggTaaC | N->G | variant | 299 |
| | | | ggTgaC | NN->GD | | |
| | | | ggTggC | NN->GG | | |
| EMR2-11 | CTTGGCCAATAACACCATCC (SEQ ID NO: 11) | AGG | TGGCCg | N->D | Missense | 298 |
| | | | | | variant | |
| EMR2-12 | CAGAGACTACAAGCCAGGCT (SEQ ID NO: 12) | TGG | GgGaCT | R RD->RG | Synonymous | 290, |
| | | | GgGgCT | | variant/ | 291 |
| | | | | | missense | |
| | | | | | variant | |
| EMR2-59 | CTGGGCAGAGACTACAAGCC (SEQ ID NO: 13) | AGG | GGGCgG | R->G | Missense | 290 |
| | | | | | variant | |
| EMR2-60 | TGTAGTCTCTGCCCAGGTCC (SEQ ID NO: 265) | TGG | TgGTCT | Y->H | Missense | 292 |
| | | | | | variant | |

TABLE 7
Exemplary sequences of exemplary gRNAs targeted to human EMR2 are provided.

| | | | SEQ | | | | CBE | ABE |
|---|---|---|---|---|---|---|---|---|
| Guide | Amino | Target Domain | ID | | | | resulting | resulting |
| Name | Acid | Sequence | NO: | Exon | | Codon | edit | edit |
| EMR2-12 | 292 | CAGAGACTACAAGCCAGGCT | 12 | 10 | NGG | TAC | GAGACTA | GaGaCTa |
| EMR2-65 | 292 | GAGACTACAAGCCAGGCTTG | 190 | 10 | NNNRRT | TAC | GACTACA | GaCTaCa |
| EMR2-72 | 296 | GGCtTGGCCAATAACACCAT | 211 | 10 | sgNNNRRT | TTG | CTTGGc | CTTGGC |
| EMR2-11 | 298 | CTTGGCCaATAACACCATCC | 11 | 10 | NGG | AAT | TGGccA | TGGCCa |
| EMR2-10 | 299 | CCAATAACACCATCCAGGTA | 10 | 10 | NGG | AAC | AATAACA | aaTaaCa |
| EMR2-77 | 299 | CAATAACACCATCCAGGTAA | 226 | 10 | NGA | AAC | ATAACAC | aTaaCaC |
| EMR2-8 | 303 | CCAGAGCATCTTACAGGCGC | 8 | 11 | NGG | AGC | AGAGCAT | aGaGCaT |
| EMR2-78 | 303 | CAGAGCATCTTACAGGCGCT | 229 | 11 | NGA | AGC | GAGCATc | GaGCaTC |
| EMR2-67 | 303 | TCCAGAGCATCTTACAGGCG | 185 | 11 | NNGRRT | AGC | CAGAGCA | CaGaGCa |
| EMR2-67 | 303 | TCCAGAGCATCTTACAGGCG | 185 | 11 | NNNRRT | AGC | CAGAGCA | CaGaGCa |
| EMR2-8 | 304 | CCAGAGCaTCTTACAGGCGC | 8 | 11 | NGG | ATC | AGAGCA | aGaGCa |
| EMR2-78 | 304 | CAGAGCaTCTTACAGGCGCT | 229 | 11 | NGA | ATC | GAGCAT | GaGCaT |
| EMR2-81 | 304 | AGCaTCTTACAGGCGCTGGA | 238 | 11 | NGAG | ATC | CATCTT | CaTCTT |
| EMR2-81 | 305 | AGCATCtTACAGGCGCTGGA | 238 | 11 | NGA | TTA | CATCTT | CaTCTT |
| EMR2-81 | 305 | AGCATCtTACAGGCGCTGGA | 238 | 11 | NGAG | TTA | CATCTT | CaTCTT |
| EMR2-55 | 307 | ACAGgCGCTGGATGAGCTGC | 49 | 11 | NGG | GCG | AGGcGc | aGGCGC |
| EMR2-79 | 307 | CAGgCGCTGGATGAGCTGCT | 232 | 11 | NGA | GCG | GGcGcT | GGCGCT |
| EMR2-79 | 307 | CAGgCGCTGGATGAGCTGCT | 232 | 11 | NGAG | GCG | GGcGcT | GGCGCT |
| EMR2-91 | 308 | GGCGCTGGATGAGCTGCTGG | 181 | 11 | NGG | CTG | CGCTGG | CGCTGG |
| EMR2-79 | 308 | CAGGCGCTGGATGAGCTGCT | 232 | 11 | NGA | CTG | GGcGcT | GGCGCT |
| EMR2-79 | 308 | CAGGCGCTGGATGAGCTGCT | 232 | 11 | NGAG | CTG | GGcGcT | GGCGCT |
| EMR2-73 | 312 | TGAGCTGCTGGAGGCCCCTG | 214 | 11 | NGG | CTG | AGCTGCT | aGCTGCT |
| EMR2-92 | 312 | GAGCTGCTGGAGGCCCCTGG | 324 | 11 | NGA | CTG | GcTGcTG | GCTGCTG |
| EMR2-74 | 329 | CTGTGTGGCCAGTCACCTGC | 217 | 11 | NGG | GTG | GTGTGGC | GTGTGGC |
| EMR2-80 | 329 | TGTgTGGCCAGTCACCTGCT | 235 | 11 | NGA | GTG | TGTGGCC | TGTGGCC |
| EMR2-73 | 329 | ACTGTgTGGCCAGTCACCTG | 242 | 11 | NNGRRT | GTG | TGTGTGG | TGTGTGG |
| EMR2-75 | 332 | AGTCACCTGCTGGATGGCCT | 220 | 11 | NGA | CAC | TcAccTG | TCaCCTG |
| EMR2-75 | 332 | AGTCACCTGCTGGATGGCCT | 220 | 11 | NGAG | CAC | TcAccTG | TCaCCTG |
| EMR2-76 | 340 | AGAGGATgTCCTCAGAGGCC | 223 | 11 | NGA | GTC | AGGATGT | aGGaTGT |
| EMR2-76 | 340 | AGAGGATgTCCTCAGAGGCC | 223 | 11 | NGAG | GTC | AGGATGT | aGGaTGT |
| EMR2-82 | 527 | CCCACtACGATGTGCAGGTG | 243 | 14 | NGA | TAC | CACTAcG | CaCTaCG |
| EMR2-93 | 527 | GGCCCACtACGATGTGCAGG | 321 | 14 | NGAG | TAC | cccAcTA | CCCaCTa |
| EMR2-94 | 527 | GTCTCACCTGCACATCGTAG | 322 | 14 | NGG | TAC | | |
| EMR2-95 | 527 | TCTCACCTGCACATCGTAGT | 323 | 14 | NGG | TAC | | |
| EMR2-83 | 708 | CTCTCtGGATTTTGAAAAAC | 247 | 18 | NGA | TGG | cTcTGGA | CTCTGGa |

In Table 7, the lowercase nucleotide refers to the edited nucleotide.

In some embodiments, the gRNA targets most PAM sequences and has reduced PAM sequence specificity. In some embodiments, such a gRNA is referred to as “PAMless” or “near PAMless.”

TABLE 8
Exemplary sequences of “PAMless” gRNAs targeted to human EMR2 are provided.
For each target site, the first sequence represents the DNA target domain
sequence, the second sequence represents the reverse complement thereof,
and the third sequence represents an exemplary targeting domain sequence
of a gRNA that can be used to target the respective target site.

| | | | | | CBE | ABE |
|---|---|---|---|---|---|---|
| Guide | Amino | | | | resulting | resulting |
| Name | Acid | Target Domain Sequence | Exon | Codon | edit | edit |
| EMR2- | 292 | GAGACtACAAGCCAGGCTTG (SEQ ID NO: 190) | 10 | TAC | GACTACA | GaCTaCa |
| 65 | | CAAGCCTGGCTTGTAGTCTC (SEQ ID NO: 191) | | | | |
| | | GAGACuACAAGCCAGGCUUG (SEQ ID NO: 192) | | | | |
| EMR2- | 294 | ACAAGCCAGGCTTGGCCAAT (SEQ ID NO: 248) | 10 | CCA | AAGccA | aaGCCa |
| 84 | | ATTGGCCAAGCCTGgCTTGT (SEQ ID NO: 267) | | | | |
| | | ACAAGCCAGGCUUGGCCAAU (SEQ ID NO: 268) | | | | |
| EMR2- | 295 | AGCCAgGCTTGGCCAATAAC (SEQ ID NO: 269) | 10 | GGC | ccAGGc | CCaGGC |
| 85 | | GTTATTGGCCAAGCCTGGCT (SEQ ID NO: 270) | | | | |
| | | AGCCAgGCUUGGCCAAUAAC (SEQ ID NO: 271) | | | | |
| EMR2- | 296 | CAGGCtTGGCCAATAACACC (SEQ ID NO: 193) | 10 | TTG | GGCTTGG | GGCTTGG |
| 66 | | GGTGTTATTGGCCAAGCCTG (SEQ ID NO: 194) | | | | |
| | | CAGGCuUGGCCAAUAACACC (SEQ ID NO: 195) | | | | |
| EMR2- | 298 | TGGCCaATAACACCATCCAG (SEQ ID NO: 272) | 10 | AAT | GccAAT | GCCaaT |
| 86 | | CTGGATGGTGTTATtGGCCA (SEQ ID NO: 273) | | | | |
| | | UGGCCaAUAACACCAUCCAG (SEQ ID NO: 274) | | | | |
| EMR2- | 299 | CCAATaACACCATCCAGGTA (SEQ ID NO: 10) | 10 | AAC | AATAACA | aa TaaCa |
| 10 | | TACCTGGATGGTGTtATTGG (SEQ ID NO: 39) | | | | |
| | | CCAAUaACACCAUCCAGGUA (SEQ ID NO: 68) | | | | |
| EMR2- | 303 | TCCAGaGCATCTTACAGGCG (SEQ ID NO: 185) | 11 | AGC | CAGAGCA | CaGaGCa |
| 67 | | CGCCTGTAAGATGCTCTGGA (SEQ ID NO: 197) | | | | |
| | | UCCAGaGCAUCUUACAGGCG (SEQ ID NO: 184) | | | | |
| EMR2- | 304 | AGAGCaTCTTACAGGCGCTG (SEQ ID NO: 275) | 11 | ATC | AGcATc | aGCaTC |
| 87 | | CAGCGCCTGTAAGAtGCTCT (SEQ ID NO: 276) | | | | |
| | | AGAGCaUCUUACAGGCGCUG (SEQ ID NO: 277) | | | | |
| EMR2- | 305 | GCATCtTACAGGCGCTGGAT (SEQ ID NO: 278) | 11 | TTA | ATCTTA | aTCTTa |
| 88 | | ATCCAGCGCCTGTAaGATGC (SEQ ID NO: 279) | | | | |
| | | GCAUCuUACAGGCGCUGGAU (SEQ ID NO: 280) | | | | |
| EMR2- | 306 | TCTTACAGGCGCTGGATGAG (SEQ ID NO: 199) | 11 | CAG | TTAcAGG | TTaCaGG |
| 68 | | CTCATCCAGCGCCTGTAAGA (SEQ ID NO: 200) | | | | |
| | | UCUUACAGGCGCUGGAUGAG (SEQ ID NO: 201) | | | | |
| EMR2- | 307 | TACAGgCGCTGGATGAGCTG (SEQ ID NO: 281) | 11 | GCG | CAGGcG | CaGGCG |
| 89 | | CAGCTCATCCAGCGCCTGTA (SEQ ID NO: 282) | | | | |
| | | UACAGgCGCUGGAUGAGCUG (SEQ ID NO: 283) | | | | |
| EMR2- | 308 | AGGCGCTGGATGAGCTGCTG (SEQ ID NO: 284) | 11 | CTG | GcGcTG | GCGCTG |
| 90 | | CAGCAGCTCATCCAgCGCCT (SEQ ID NO: 285) | | | | |
| | | AGGCGCUGGAUGAGCUGCUG (SEQ ID NO: 286) | | | | |
| EMR2- | 312 | AGCTGCTGGAGGCCCCTGGG (SEQ ID NO: 202) | 11 | CTG | CTGCTGG | CTGCTGG |
| 69 | | CCCAGGGGCCTCCAGCAGCT (SEQ ID NO: 203) | | | | |
| | | AGCUGCUGGAGGCCCCUGGG (SEQ ID NO: 204) | | | | |
| EMR2- | 318 | GGGACCTGGAGACCCTGCCC (SEQ ID NO: 205) | 11 | CTG | GAccTGG | GaCCTGG |
| 70 | | GGGCAGGGTCTCCAGGTCCC (SEQ ID NO: 206) | | | | |
| | | GGGACCUGGAGACCCUGCCC (SEQ ID NO: 207) | | | | |
| EMR2- | 320 | TGGAGaCCCTGCCCCGCTTA (SEQ ID NO: 208) | 11 | ACC | GAGAccc | GaGaCCC |
| 71 | | TAAGCGGGGCAGGGTCTCCA (SEQ ID NO: 209) | | | | |
| | | UGGAGaCCCUGCCCCGCUUA (SEQ ID NO: 210) | | | | |
| EMR2- | 328 | AGCACtGTGTGGCCAGTCAC (SEQ ID NO: 287) | 11 | TGT | cACTGTG | CaCTGTG |
| 72 | | GTGACTGGCCACACaGTGCT (SEQ ID NO: 288) | | | | |
| | | AGCACuGUGUGGCCAGUCAC (SEQ ID NO: 289) | | | | |
| EMR2- | 329 | ACTGTgTGGCCAGTCACCTG (SEQ ID NO: 242) | 11 | GTG | TGTGTGG | TGTGTGG |
| 73 | | CAGGTGACTGGCCACACAGT (SEQ ID NO: 290) | | | | |
| | | ACUGUgUGGCCAGUCACCUG (SEQ ID NO: 291) | | | | |
| EMR2- | 331 | TGGCCaGTCACCTGCTGGAT (SEQ ID NO: 292) | 11 | AGT | GccAGTc | GCCaGTC |
| 74 | | ATCCAGCAGGTGACtGGCCA (SEQ ID NO: 293) | | | | |
| | | UGGCCaGUCACCUGCUGGAU (SEQ ID NO: 294) | | | | |
| EMR2- | 332 | CCAGTcACCTGCTGGATGGC (SEQ ID NO: 295) | 11 | CAC | AGTcAcc | aGTCaCC |
| 75 | | GCCATCCAGCAGGTgACTGG (SEQ ID NO: 296) | | | | |
| | | CCAGUcACCUGCUGGAUGGC (SEQ ID NO: 297) | | | | |
| EMR2- | 335 | TGCTGgATGGCCTAGAGGAT (SEQ ID NO: 298) | 11 | GAT | CTGGATG | CTGGaTG |
| 76 | | ATCCTCTAGGCCATCCAGCA (SEQ ID NO: 299) | | | | |
| | | UGCUGgAUGGCCUAGAGGAU (SEQ ID NO: 300) | | | | |
| EMR2- | 340 | AGGATgTCCTCAGAGGCCTG (SEQ ID NO: 301) | 11 | GTC | GATGTcc | GaTGTCC |
| 77 | | CAGGCCTCTGAGGACATCCT (SEQ ID NO: 302) | | | | |
| | | AGGAUgUCCUCAGAGGCCUG (SEQ ID NO: 303) | | | | |
| EMR2- | 347 | GCAAGaACCTTTCCAATGGG (SEQ ID NO: 304) | 11 | AAC | AAGAAcc | aaGaaCC |
| 78 | | CCCATTGGAAAGGTtCTTGC (SEQ ID NO: 305) | | | | |
| | | GCAAGaACCUUUCCAAUGGG (SEQ ID NO: 306) | | | | |
| EMR2- | 124 | AATGTCAGCAGAACCCAAGG (SEQ ID NO: 307) | 6 | CAG | TGTcAGc | TGTCaGC |
| 79 | | CCTTGGGTTCTGCTgACATT (SEQ ID NO: 308) | | | | |
| | | AAUGUCAGCAGAACCCAAGG (SEQ ID NO: 309) | | | | |
| EMR2- | 146 | CGTGCCAGTGCCTGCCTGGC (SEQ ID NO: 310) | 6 | CAG | TGccAGT | TGCCaGT |
| 80 | | GCCAGGCAGGCACTgGCACG (SEQ ID NO: 311) | | | | |
| | | CGUGCCAGUGCCUGCCUGGC (SEQ ID NO: 312) | | | | |
| EMR2- | 132 | GTAAAaGCTACGGCACCTGC (SEQ ID NO: 313) | 6 | AGC | AAAAGCT | aaaaGCT |
| 81 | | GCAGGTGCCGTAGCETTTAC (SEQ ID NO: 314) | | | | |
| | | GUAAAaGCUACGGCACCUGC (SEQ ID NO: 315) | | | | |
| EMR2- | 527 | CCCACtACGATGTGCAGGTG (SEQ ID NO: 243) | 14 | TAC | CACTAcG | CaCTaCG |
| 82 | | CACCTGCACATCGTaGTGGG (SEQ ID NO: 316) | | | | |
| | | CCCACuACGAUGUGCAGGUG (SEQ ID NO: 317) | | | | |
| EMR2- | 708 | CTCTCtGGATTTTGAAAAAC (SEQ ID NO: 247) | 18 | TGG | CTCTGGA | CTCTGGa |
| 83 | | GTTTTTCAAAATCCaGAGAG (SEQ ID NO: 318) | | | | |
| | | CUCUCuGGAUUUUGAAAAAC (SEQ ID NO: 319) | | | | |

In Table 8, the lowercase nucleotide refers to the edited nucleotide.

In some embodiments, any of the gRNAs provided in Table 7 may be used with an ABE or a CBE. In some embodiments, any of the gRNAs provided in Table 7 may be used with a SpRY Cas9 with NRN PAM.

A representative amino acid sequence of full length EMR2 is provided by UniProtKB/Swiss-Prot Accession No. Q9UHX3-1, shown below.

(SEQ ID NO: 167)
MGGRVFLVFLAFCVWLTLPGAETQDSRGCARWCPQDSSCVNATAC
RCNPGFSSFSEIITTPMETCDDINECATLSKVSCGKFSDCWNTEG
SYDCVCSPGYEPVSGAKTFKNESENTCQDVDECQQNPRLCKSYGT
CVNTLGSYTCQCLPGFKLKPEDPKLCTDVNECTSGQNPCHSSTHC
LNNVGSYQCRCRPGWQPIPGSPNGPNNTVCEDVDECSSGQHQCDS
STVCFNTVGSYSCRCRPGWKPRHGIPNNQKDTVCEDMTFSTWTPP
PGVHSQTLSRFFDKVQDLGRDYKPGLANNTIQSILQALDELLEAP
GDLETLPRLQQHCVASHLLDGLEDVLRGLSKNLSNGLLNFSYPAG
TELSLEVQKQVDRSVTLRQNQAVMQLDWNQAQKSGDPGPSVVGLV
SIPGMGKLLAEAPLVLEPEKQMLLHETHQGLLQDGSPILLSDVIS
AFLSNNDTQNLSSPVTFTFSHRSVIPRQKVLCVFWEHGQNGCGHW
ATTGCSTIGTRDTSTICRCTHLSSFAVLMAHYDVQEEDPVLTVIT
YMGLSVSLLCLLLAALTFLLCKAIQNTSTSLHLQLSLCLFLAHLL
FLVAIDQTGHKVLCSIIAGTLHYLYLATLTWMLLEALYLFLTARN
LTVVNYSSINRFMKKLMFPVGYGVPAVTVAISAASRPHLYGTPSR
CWLQPEKGFIWGFLGPVCAIFSVNLVLFLVTLWILKNRLSSLNSE
VSTLRNTRMLAFKATAQLFILGCTWCLGILQVGPAARVMAYLFTI
INSLQGVFIFLVYCLLSQQVREQYGKWSKGIRKLKTESEMHTLSS
SAKADTSKPSTVN

A representative DNA sequence of EMR2 is provided by NCBI Reference Sequence No. NG_047146.1.

Some aspects of this disclosure provide genetically engineered cells comprising a modification in their genome that results in a loss of expression of EMR2, or expression of a variant form of EMR2 that is not recognized by an immunotherapeutic agent targeting EMR2. In some embodiments, the modification in the genome of the cell is a mutation in a genomic sequence encoding EMR2. In some embodiments, the modification is effected via genome editing, e.g., using a Cas nuclease and a gRNA targeting a EMR2 target site provided herein or comprising a targeting domain sequence provided herein.

While the compositions, methods, strategies, and treatment modalities provided herein may be applied to any cell or cell type, some exemplary cells and cell types that are particularly suitable for genomic modification in the EMR2 gene according to aspects of this invention are described in more detail herein. The skilled artisan will understand, however, that the provision of such examples is for the purpose of illustrating some specific embodiments, and additional suitable cells and cell types will be apparent to the skilled artisan based on the present disclosure, which is not limited in this respect.

Some aspects of this disclosure provide genetically engineered hematopoietic cells comprising a modification in their genome that results in a loss of expression of EMR2, or expression of a variant form of EMR2 that is not recognized by an immunotherapeutic agent targeting EMR2. In some embodiments, the genetically engineered cells comprising a modification in their genome results in reduced cell surface expression of EMR2 and/or reduced binding by an immunotherapeutic agent targeting EMR2, e.g., as compared to a hematopoietic cell of the same cell type but not comprising a genomic modification. In some embodiments, a hematopoietic cell is a hematopoietic stem cell (HSC). In some embodiments, the hematopoietic cell is a hematopoietic progenitor cell (HPC). In some embodiments, the hematopoietic cell is a hematopoietic stem or progenitor cell.

In some embodiments, the cells are CD34+. In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is selected from the group consisting of an embryonic stem cell (ESC), an induced pluripotent stem cell (iPSC), a mesenchymal stem cell, or a tissue-specific stem cell.

In some embodiments, the cells are comprised in a population of cells which is characterized by the ability to engraft EMR2-edited hematopoietic stem cells in the bone marrow of a recipient and to generate differentiated progeny of all blood lineage cell types in the recipient. In some embodiments, the cell population is characterized by the ability to engraft EMR2-edited hematopoietic stem cells in the bone marrow of a recipient at an efficiency of at least 50%. In some embodiments, the cell population is characterized by the ability to engraft EMR2-edited hematopoietic stem cells in the bone marrow of a recipient at an efficiency of at least 60%. In some embodiments, the cell population is characterized by the ability to engraft EMR2-edited hematopoietic stem cells in the bone marrow of a recipient at an efficiency of at least 70%. In some embodiments, the cell population is characterized by the ability to engraft EMR2-edited hematopoietic stem cells in the bone marrow of a recipient at an efficiency of at least 80%. In some embodiments, the cell population is characterized by the ability to engraft EMR2-edited hematopoietic stem cells in the bone marrow of a recipient at an efficiency of at least 90%. In some embodiments, the cell population comprises EMR2-edited hematopoietic stem cells that are characterized by a differentiation potential that is equivalent to the differentiation potential of non-edited hematopoietic stem cells.

In some embodiments, a hematopoietic cell (e.g., an HSC or HPC) comprising a modification in its genome that results in a loss of expression of EMR2, or expression of a variant form of EMR2 that is not recognized by an immunotherapeutic agent targeting EMR2, is created using a nuclease and/or a gRNA targeting human EMR2 as described herein. It will be understood that such a cell can be created by contacting the cell with the nuclease and/or the gRNA, or the cell can be the daughter cell of a cell that was contacted with the nuclease and/or gRNA. In some embodiments, a cell described herein (e.g., a genetically engineered HSC or HPC) is capable of populating the HSC or HPC niche and/or of reconstituting the hematopoietic system of a subject. In some embodiments, a cell described herein (e.g., an HSC or HPC) is capable of one or more of (e.g., all of): engrafting in a human subject, producing myeloid lineage cells, and producing and lymphoid lineage cells. In some preferred embodiments, a genetically engineered hematopoietic cell provided herein, or its progeny, can differentiate into all blood cell lineages, preferably without any differentiation bias as compared to a hematopoietic cell of the same cell type, but not comprising a genomic modification that results in a loss of expression of EMR2, or expression of a variant form of EMR2 that is not recognized by an immunotherapeutic agent targeting EMR2.

It will be understood that, upon engrafting donor cells into a recipient host organism, the relative levels of the engrafted donor cells (and descendants thereof) and the host cells, e.g., in a given niche (e.g., bone marrow), are important for physiological and/or therapeutic outcomes for the host organism. The level of engrafted donor cells or descendants thereof relative to host cells in a given tissue or niche is referred to herein as chimerism. In some embodiments, a cell described herein (e.g., an HSC or HPC) is capable of engrafting in a human subject and does not exhibit any difference in chimerism as compared to a hematopoietic cell of the same cell type, but not comprising a genomic modification that results in a loss of expression of EMR2, or expression of a variant form of EMR2 that is not recognized by an immunotherapeutic agent targeting EMR2. In some embodiments, a cell described herein (e.g., an HSC or HPC) is capable of engrafting in a human subject exhibits no more than a 1, 2, 5, 10, 15, 20, 25, 30, 35, 40, 45, or 50% difference in chimerism as compared to a hematopoietic cell of the same cell type, but not comprising a genomic modification that results in a loss of expression of EMR2, or expression of a variant form of EMR2 that is not recognized by an immunotherapeutic agent targeting EMR2.

In some embodiments, a genetically engineered cell provided herein comprises only one genomic modification, e.g., a genomic modification that results in a loss of expression of EMR2, or expression of a variant form of EMR2 that is not recognized by an immunotherapeutic agent targeting EMR2. It will be understood that the gene editing methods provided herein may result in genomic modifications in one or both alleles of a target gene. In some embodiments, genetically engineered cells comprising a genomic modification in both alleles of a given genetic locus are preferred.

In some embodiments, a genetically engineered cell provided herein comprises two or more genomic modifications, e.g., one or more genomic modifications in addition to a genomic modification that results in a loss of expression of EMR2, or expression of a variant form of EMR2 that is not recognized by an immunotherapeutic agent targeting EMR2.

In some embodiments, a genetically engineered cell provided herein comprises a genomic modification that results in a loss of expression of EMR2, or expression of a variant form of EMR2 that is not recognized by an immunotherapeutic agent targeting EMR2, and further comprises an expression construct that encodes a chimeric antigen receptor, e.g., in the form of an expression construct encoding the CAR integrated in the genome of the cell. In some embodiments, the CAR comprises a binding domain, e.g., an antibody fragment, that binds EMR2.

Some aspects of this disclosure provide genetically engineered immune effector cells comprising a modification in their genome that results in a loss of expression of EMR2, or expression of a variant form of EMR2 that is not recognized by an immunotherapeutic agent targeting EMR2. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell. In some embodiments, the immune effector cell does not express an endogenous transgene, e.g., a transgenic protein. In some embodiments, the immune effector cell expresses a chimeric antigen receptor (CAR). In some embodiments, the immune effector cell expresses a CAR targeting EMR2. In some embodiments, the immune effector cell does not express a CAR targeting EMR2.

In some embodiments, a genetically engineered cell provided herein comprises a genomic modification that results in a loss of expression of EMR2, or expression of a variant form of EMR2 that is not recognized by an immunotherapeutic agent targeting EMR2 and does not comprise an expression construct that encodes an exogenous protein, e.g., does not comprise an expression construct encoding a CAR.

In some embodiments, a genetically engineered cell provided herein expresses substantially no EMR2 protein, e.g., expresses no EMR2 protein that can be measured by a suitable method, such as an immunostaining method. In some embodiments, a genetically engineered cell provided herein expresses substantially no wild-type EMR2 protein but expresses a mutant EMR2 protein variant, e.g., a variant not recognized by an immunotherapeutic agent targeting EMR2, e.g., a CAR-T cell therapeutic, or an anti-EMR2 antibody, antibody fragment, or antibody-drug conjugate (ADC).

In some embodiments, the genetically engineered cells provided herein are hematopoietic cells, e.g., hematopoietic stem cells, hematopoietic progenitor cells (HPCs), hematopoietic stem or progenitor cells Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of one or more cell surface markers, e.g., CD34 (e.g., CD34+), which can be used for the identification and/or isolation of HSCs, and absence of cell surface markers associated with commitment to a cell lineage. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a population of genetically engineered cells described herein comprises a plurality of genetically engineered hematopoietic stem cells. In some embodiments, a population of genetically engineered cells described herein comprises a plurality of genetically engineered hematopoietic progenitor cells. In some embodiments, a population of genetically engineered cells described herein comprises a plurality of genetically engineered hematopoietic stem cells and a plurality of genetically engineered hematopoietic progenitor cells.

In some embodiments, the genetically engineered HSCs are obtained from a subject, such as a human subject. Methods of obtaining HSCs are described, e.g., in PCT Publication No. US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g. heterogeneous population of genetically engineered cells containing different EMR2 mutations. In some embodiments, at least 40%, at least 50%, at least 60%, at least 70%, at least 75%, at least 80%, at least 85%, at least 90%, or at least 95% of copies of a gene encoding EMR2 in the population of genetically engineered cells comprise a mutation effected by a genome editing approach described herein, e.g., by a CRISPR/Cas system using a gRNA provided herein. By way of example, a population of genetically engineered cells can comprise a plurality of different EMR2 mutations and each mutation of the plurality may contribute to the percent of copies of EMR2 in the population of cells that have a mutation.

In some embodiments, the expression of EMR2 on the genetically engineered hematopoietic cell is compared to the expression of EMR2 on a naturally occurring hematopoietic cell (e.g., a wild-type counterpart). In some embodiments, the genetic engineering results in a reduction in the expression level of EMR2 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of EMR2 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of EMR2 as compared to a naturally occurring hematopoietic cell (e.g., a wild-type counterpart).

In some embodiments, the genetic engineering as described herein, e.g., using a gRNA targeting EMR2 as described herein, results in a reduction in the expression level of wild-type EMR2 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type EMR2 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of EMR2 as compared to a naturally occurring hematopoietic cell (e.g., a wild-type counterpart).

In some embodiments, the genetic engineering as described herein, e.g., using a gRNA targeting EMR2 as described herein, results in a reduction in the expression level of wild-type lineage-specific cell surface antigen (e.g., EMR2) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell surface antigen measured or expected in a plurality of non-engineered cells from the same subject. In some embodiments, the suitable control comprises the level of the wild-type lineage-specific cell surface antigen measured or expected in a plurality of cells from a healthy subject. In some embodiments, the suitable control comprises the level of the wild-type lineage-specific cell surface antigen measured or expected in a population of cells from a pool of healthy individuals (e.g., 10, 20, 50, or 100 individuals). In some embodiments, the suitable control comprises the level of the wild-type lineage-specific cell surface antigen measured or expected in a subject in need of a treatment described herein, e.g., an anti-EMR2 therapy, e.g., wherein the subject has a cancer, wherein cells of the cancer express EMR2.

In some embodiments, a method of genetically engineering cells described herein comprises a step of providing a wild-type cell, e.g., a wild-type hematopoietic stem or progenitor cell. In some embodiments, the wild-type cell is an un-edited cell comprising (e.g., expressing) two functional copies of a gene encoding EMR2. In some embodiments, the cell comprises a EMR2 gene sequence as provided in NCBI Reference Sequence No. NG_047146.1. In some embodiments, the cell comprises a EMR2 gene sequence encoding a EMR2 protein that is encoded in the sequence provided by NCBI Reference Sequence No. NG_047146.1, e.g., the EMR2 gene sequence may comprise one or more silent mutations relative to the sequence provided in NCBI Reference Sequence No. NG_047146.1. In some embodiments, the cell used in the method is a naturally occurring cell or a non-engineered cell. In some embodiments, the wild-type cell expresses EMR2, or gives rise to a more differentiated cell that expresses EMR2 at a level comparable to (or within 90%-110%, 80%-120%, 70%-130%, 60-140%, or 50%-150% of) a cell line expressing EMR2.

In some embodiments, the wild-type cell binds an antibody that binds EMR2 (e.g., an anti-EMR2 antibody), or gives rise to a more differentiated cell that binds such an antibody at a level comparable to (or within 90%-110%, 80%-120%, 70%-130%, 60-140%, or 50%-150% of) binding of the antibody to a cell line expressing EMR2, e.g., L1236, L428, KM-H2, and L591). Antibody binding may be measured, for example, by flow cytometry or immunohistochemistry.

Dual gRNA Compositions and Uses Thereof

In some embodiments, a gRNA provided herein (e.g., a gRNA provided in any of Tables 1-8) can be used in combination with a second gRNA, e.g., for targeting a CRISPR/Cas nuclease to two sites in a genome. For instance, in some embodiments it may desired to produce a hematopoietic cell that is deficient for EMR2 and a second lineage-specific cell surface antigen, e.g., CD33, CD123, CLL-1, CD19, CD30, CD5, CD6, CD7, CD38, so that the cell can be resistant to two agents: an anti-EMR2 agent and an agent targeting the second lineage-specific cell surface antigen. In some embodiments, it is desirable to contact a cell with two different gRNAs that target different sites of EMR2, e.g., in order to make two cuts and create a deletion or an insertion between the two cut sites. Accordingly, the disclosure provides various combinations of gRNAs and related CRISPR systems, as well as cells created by genome editing methods using such combinations of gRNAs and related CRISPR systems. In some embodiments, the EMR2 gRNA binds a different nuclease than the second gRNA. For example, in some embodiments, the EMR2 gRNA may bind Cas9 and the second gRNA may bind Cas12a, or vice versa. For example, in some embodiments, the EMR2 gRNA may bind a base editor (e.g., ABE, CBE) and the second gRNA may bind another RNA-guided nuclease, or vice versa.

In some embodiments, the first gRNA is an EMR2 gRNA provided herein (e.g., a gRNA provided in any of Tables 1-8 or a variant thereof) and the second gRNA targets a lineage-specific cell-surface antigen chosen from: BCMA, CD19, CD20, CD30, ROR1, B7H6, B7H3, CD23, CD33, CD38, C-type lectin like molecule-1, CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and CD26.

In some embodiments, the first gRNA is an EMR2 gRNA provided herein (e.g., a gRNA provided in any one of Tables 1-8 or a variant thereof) and the second gRNA targets a lineage-specific cell-surface antigen associated with a neoplastic or malignant disease or disorder, e.g., with a specific type of cancer, such as, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is an EMR2 gRNA provided herein (e.g., a gRNA provided in any one of Tables 1-8 or a variant thereof) and the second gRNA targets a lineage-specific cell-surface antigen chosen from: CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the second gRNA is a gRNA disclosed in any of PCT Publication Nos. WO2017/066760, WO2019/046285, WO/2018/160768, or Borot et al. PNAS (2019) 116 (24):11978-11987, each of which is incorporated herein by reference in its entirety.

### Methods of Administration to Subjects in Need Thereof

Some aspects of this disclosure provide methods comprising administering an effective number of genetically engineered cells as described herein, comprising a modification in their genome that results in a loss of expression of EMR2, or expression of a variant form of EMR2 that is not recognized by an immunotherapeutic agent targeting EMR2, to a subject in need thereof.

A subject in need thereof is, in some embodiments, a subject undergoing or about to undergo an immunotherapy targeting EMR2. A subject in need thereof is, in some embodiments, a subject having or having been diagnosed with, a malignancy characterized by expression of EMR2 on malignant cells. In some embodiments, a subject having such a malignancy may be a candidate for immunotherapy targeting EMR2, but the risk of detrimental on-target, off-disease effects may outweigh the benefit, expected or observed, to the subject. In some such embodiments, administration of genetically engineered cells as described herein, results in an amelioration of the detrimental on-target, off-disease effects, as the genetically engineered cells provided herein are not targeted efficiently by an immunotherapeutic agent targeting EMR2.

In some embodiments, the malignancy is a hematologic malignancy, or a cancer of the blood. In some embodiments, the malignancy is a lymphoid malignancy. In general, lymphoid malignancies are associated with the inappropriate production, development, and/or function of lymphoid cells, such as lymphocytes of the T lineage or the B lineage. In some embodiments, the malignancy is characterized or associated with cells that express EMR2 on the cell surface.

As described herein, EMR2 expression has also been observed in samples from patients having acute myeloid leukemia.

In some embodiments, the malignancy is associated with aberrant T lymphocytes, such as a T-lineage cancer, e.g., a T cell leukemia or a T-cell lymphoma.

Examples of T cell leukemias and T-cell lymphomas include, without limitation, T-lineage Acute Lymphoblastic Leukemia (T-ALL), Hodgkin's lymphoma, or a non-Hodgkin's lymphoma, acute lymphoblastic leukemia (ALL), chronic lymphocytic leukemia (CLL), large granular lymphocytic leukemia, adult T-cell leukemia/lymphoma (ATLL), T-cell prolymphocytic leukemia (T-PLL), T-cell chronic lymphocytic leukemia, T-prolymphocytic leukemia, T-cell lymphocytic leukemia, peripheral T-cell lymphoma not otherwise specified (PTCL-NOS), enteropathy associated T-cell lymphoma, B-cell chronic lymphocytic leukemia, mantle cell lymphoma, peripheral T-cell lymphoma (PTCL), anaplastic large-cell lymphoma, cutaneous T-cell lymphoma, angioimmunoblastic lymphoma, anaplastic large cell lymphoma, enteropathy-type T-cell lymphoma, hematosplenic gamma-delta T-cell lymphoma, lymphoblastic lymphoma, or hairy cell leukemia. In some embodiments, the malignancy is acute myeloid leukemia.

As described herein, EMR2 expression is associated with bladder carcinoma, breast carcinoma, colorectal carcinoma, gastric and esophageal carcinoma, and glioblastoma. See, e.g., Safaee et al. Onc. Rev. (2014) 8(242): 20-24.

In some embodiments, the malignancy is associated with aberrant epithelia, e.g., a carcinoma.

Examples of carcinomas include, without limitation, adrenocortical carcinoma, adenocarcinoma, basal cell carcinoma of the skin, breast carcinoma, ovarian carcinoma, colorectal carcinoma, gastric and esophageal carcinoma, invasive ductal carcinoma, pancreatic carcinoma, renal cell carcinoma, and squamous cell carcinoma.

In some embodiments, the malignancy is a glioblastoma.

Also within the scope of the present disclosure are malignancies that are considered to be relapsed and/or refractory, such as relapsed or refractory carcinoma. A subject in need thereof is, in some embodiments, a subject undergoing or that will undergo an immune effector cell therapy targeting EMR2, e.g., CAR-T cell therapy, wherein the immune effector cells express a CAR targeting EMR2, and wherein at least a subset of the immune effector cells also express EMR2 on their cell surface. As used herein, the term “fratricide” refers to self-killing. For example, cells of a population of cells kill or induce killing of cells of the same population. In some embodiments, cells of the immune effector cell therapy kill or induce killing of other cells of the immune effector cell therapy. In such embodiments, fratricide ablates a portion of or the entire population of immune effector cells before a desired clinical outcome, e.g., ablation of malignant cells expressing EMR2 within the subject, can be achieved. In some such embodiments, using genetically engineered immune effector cells, as provided herein, e.g., immune effector cells that do not express EMR2 or do not express a EMR2 variant recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express EMR2 or do not express a EMR2 variant recognized by the CAR, may be further modified to also express the EMR2-targeting CAR. In some embodiments, the immune effector cells may be lymphocytes, e.g., T-lymphocytes, such as, for example alpha/beta T-lymphocytes, gamma/delta T-lymphocytes, or natural killer T cells. In some embodiments, the immune effector cells may be natural killer (NK) cells.

In some embodiments, an effective number of genetically engineered cells as described herein, comprising a modification in their genome that results in a loss of expression of EMR2, or expression of a variant form of EMR2 that is not recognized by an immunotherapeutic agent targeting EMR2, is administered to a subject in need thereof, e.g., to a subject undergoing or that will undergo an immunotherapy targeting EMR2, wherein the immunotherapy is associated or is at risk of being associated with a detrimental on-target, off-disease effect, e.g., in the form of cytotoxicity towards healthy cells in the subject that express EMR2. In some embodiments, an effective number of such genetically engineered cells may be administered to the subject in combination with the anti-EMR2 immunotherapeutic agent.

It is understood that when agents (e.g., EMR2-modified cells and an anti-EMR2 immunotherapeutic agent) are administered in combination, the cells and the agent may be administered at the same time or at different times, e.g., in temporal proximity. Furthermore, the cells and the agent may be admixed or in separate volumes or dosage forms. For example, in some embodiments, administration in combination includes administration in the same course of treatment, e.g., in the course of treating a subject with an anti-EMR2 immunotherapy, the subject may be administered an effective number of genetically engineered, EMR2-modified cells concurrently or sequentially, e.g., before, during, or after the treatment, with the anti-EMR2 immunotherapy.

In some embodiments, the immunotherapeutic agent that targets EMR2 as described herein is an immune cell that expresses a chimeric antigen receptor, which comprises an antigen-binding fragment (e.g., a single-chain antibody) capable of binding to EMR2. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise a EMR2-binding antibody fragment. The antibody fragment can comprise one or more CDRs, the variable regions (or portions thereof), the constant regions (or portions thereof), or combinations of any of the foregoing.

Exemplary antibodies that recognize human EMR2 are provided, for example in Chang et al. FEBS Letters. (2003) 547(1-3):145-150; Yona et al. FASEB J. (2008). 22(3): 741-751; in PCT Publication No. WO2017/087800, which are incorporated by reference herein in their entireties.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in PCT Publication No. WO 2019/178382, and in Table 9 below.

TABLE 9
Exemplary components of a chimeric receptor

| Chimeric receptor component | Amino acid sequence |
|---|---|
| Antigen-binding fragment | Light chain-Linker-Heavy chain |
| CD28 costimulatory domain | IEVMYPPPYLDNEKSNGTIIHVKGKHLCP |
| | SPLFPGPSKPFWVLVVVGGVLACYSLLVTV |
| | AFIIFWVRSKRSRLLHSDYMNMTPRRPGPT |
| | RKHYQPYAPPRDFAAYRS (SEQ ID NO: 168) |
| CD8alpha transmembrane | IYIWAPLAGTCGVLLLSLVITLYC |
| domain | (SEQ ID NO: 169) |
| CD28 transmembrane domain | FWVLVVVGGVLACYSLLVTVAFII |
| | FWVRSKRSRLLHSDYMNMTPRR |
| | PGPTRKHYQPYAPPRDFAAYRS |
| | (SEQ ID NO: 170) |
| 4-1BB intracellular domain | KRGRKKLLYIFKQPFMRVQTTQEEDGCS |
| | CRFPEEEEGGCEL (SEQ ID NO: 171) |
| CD3ζ cytoplasmic signaling | RVKFSRSADAPAYQQGQNQLYNELNLG |
| domain | RREEYDVLDKRRGRDPEMGGKPQRRKNP |
| | QEGLYNELQKDKMAEAYSEIGMKGERRR |
| | GKGHDGLYQGLSTATKDTYDALHMQALPPR |
| | (SEQ ID NO: 172) |

In some embodiments, the number of genetically engineered cells provided herein, e.g., HSCs, HPCs, or immune effector cells that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells that are administered to a subject in need thereof is about 10⁶, about 10⁷, about 10⁸, about 10⁹, about 10¹⁰, or about 10¹¹. In some embodiments, the number of genetically engineered cells provided herein, e.g., HSCs, HPCs, or immune effector cells that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

In some embodiments, the immunotherapeutic agent that targets EMR2 is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on its cell surface (e.g., target cell), thereby resulting in death of the target cell.

Suitable antibodies and antibody fragments binding EMR2 will be apparent to those of ordinary skill in the art, and include, for example, those described in PCT Publication No. WO2017/087800, the entire contents of which are incorporated herein by reference.

Toxins or drugs compatible for use in antibody-drug conjugates are known in the art and will be evident to one of ordinary skill in the art. See, e.g., Peters et al. Biosci. Rep. (2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19. In some embodiments, the antibody-drug conjugate is belantamab mafodotin.

In some embodiments, the antibody-drug conjugate may further comprise a linker (e.g., a peptide linker, such as a cleavable linker) attaching the antibody and drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BJIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, and DSTA4637S/RG7861.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Example 1: Genetic Editing of EMR2 in Human Cells

Design of sgRNA Constructs

The target domains and gRNAs indicated in Tables 1-2 were designed by manual inspection for a PAM sequence for an applicable nuclease, e.g., Cas9, Cpf1, with close proximity to the target region and prioritized according to predicted specificity by minimizing potential off-target sites in the human genome with an online search algorithm (e.g., the Benchling algorithm, Doench et al 2016, Hsu et al 2013). All designed synthetic sgRNAs were produced with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified.

### Editing in Primary Human CD34+ HSCs

Screening of the EMR2 gRNAs was performed using the gRNAs shown in Tables 1 and 2 in HL60 cells or CD34+ cells. CD34+ HSCs derived from mobilized peripheral blood (mPB) were thawed according to manufacturer's instructions. To edit CD34+ HSCs, ˜1×10⁶ HSCs were thawed and cultured in StemSpan SFEM medium supplemented with StemSpan CC110 cocktail (StemCell Technologies) for 24-48 h before electroporation with RNP. To electroporate HSCs, 1.5×10⁵ cells were pelleted and resuspended in 20 μL electroporation solution and mixed with 10 μL Cas9 RNP.

### Genomic DNA Analysis

For all genomic analysis, DNA was harvested from cells, amplified with primers flanking the target region, purified and the allele modification frequencies were analyzed using TIDE (Tracking of Indels by Decomposition). Analyses are performed using a reference sequence from a mock-transfected sample. Parameters were set to the default maximum indel size of 10 nucleotides and the decomposition window to cover the largest possible window with high quality traces. All TIDE analyses below the detection sensitivity of 3.5% were set to 0%.

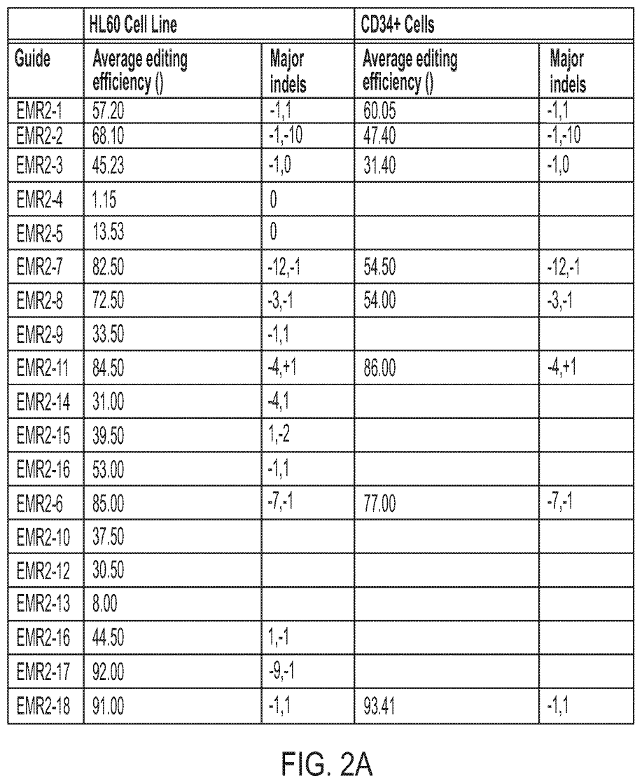

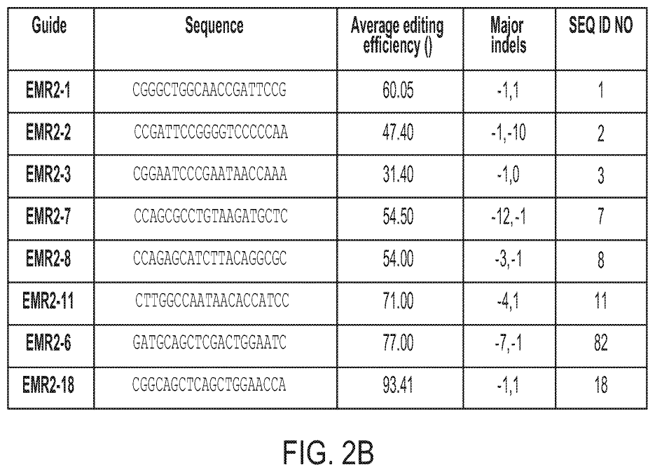

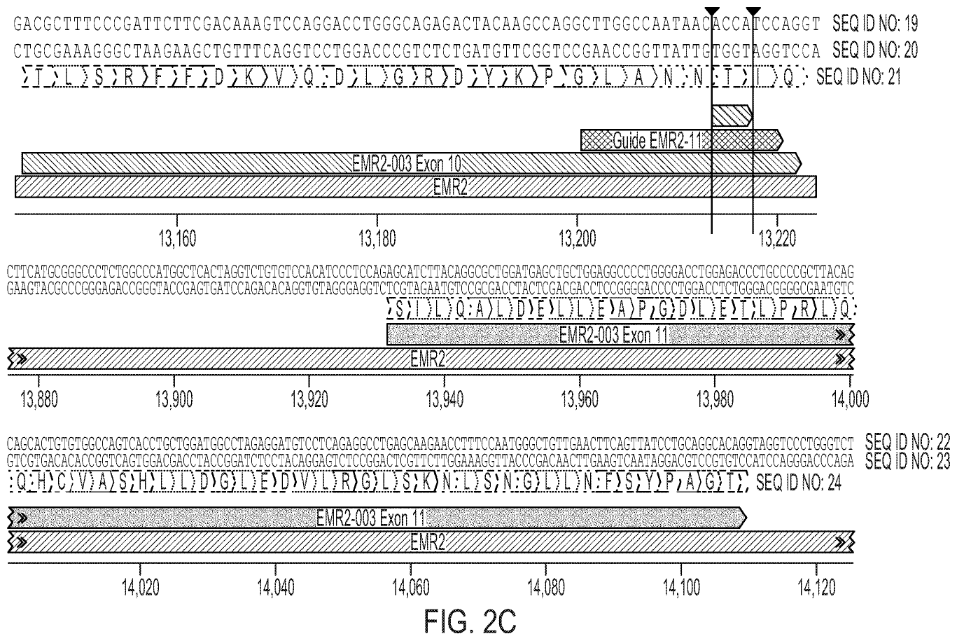

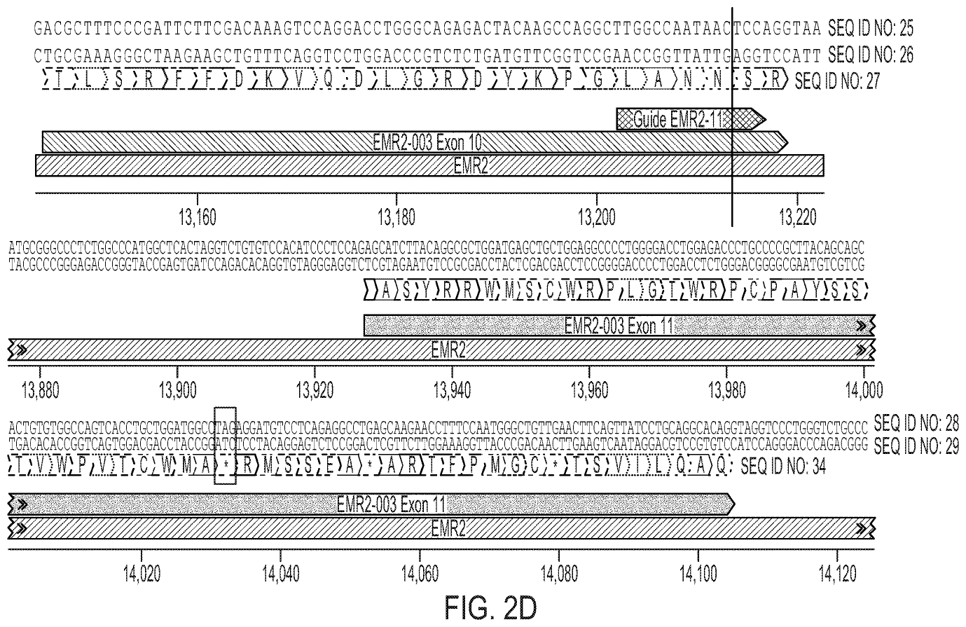

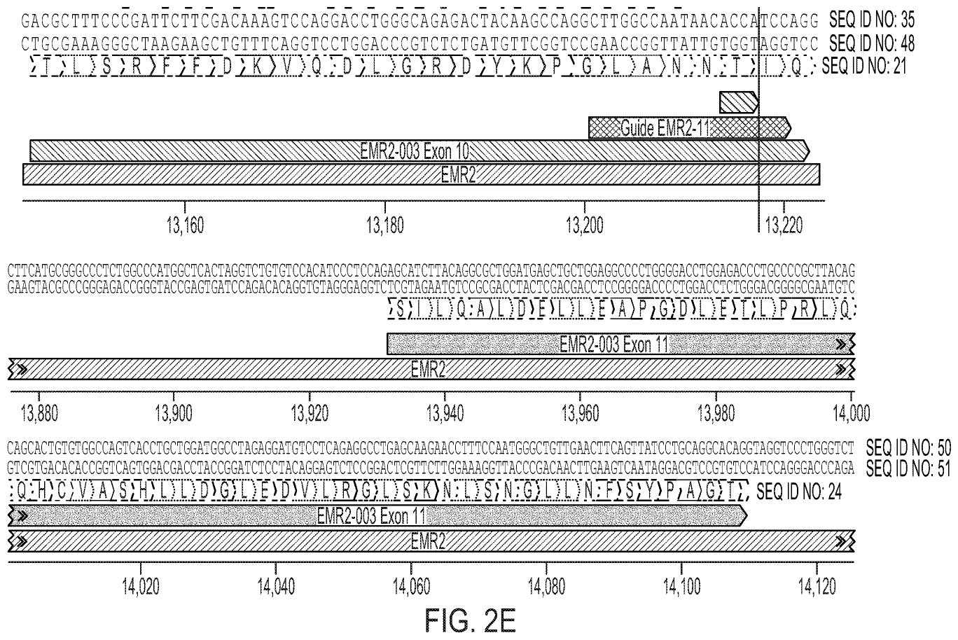

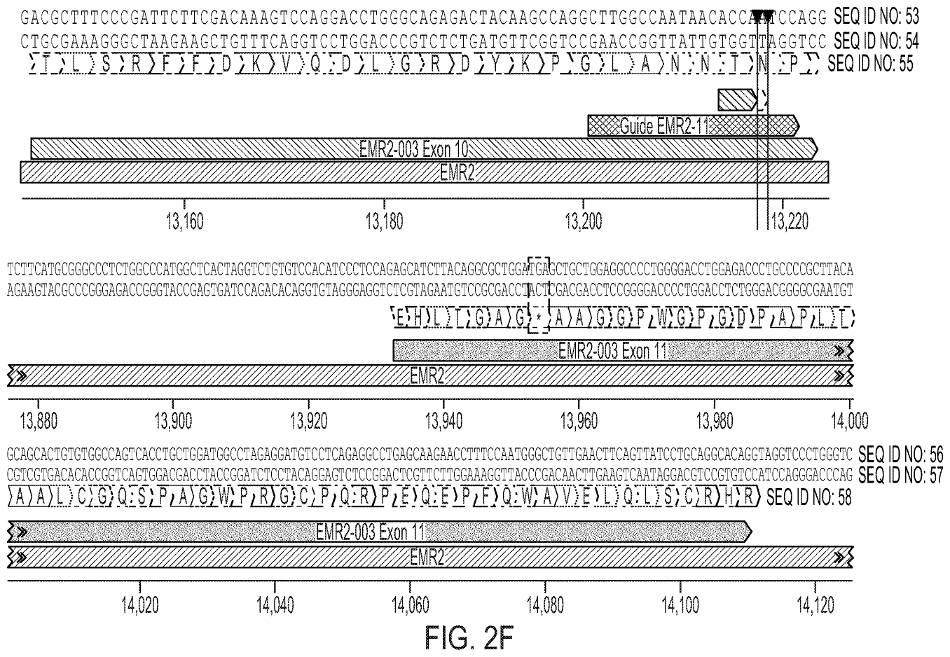

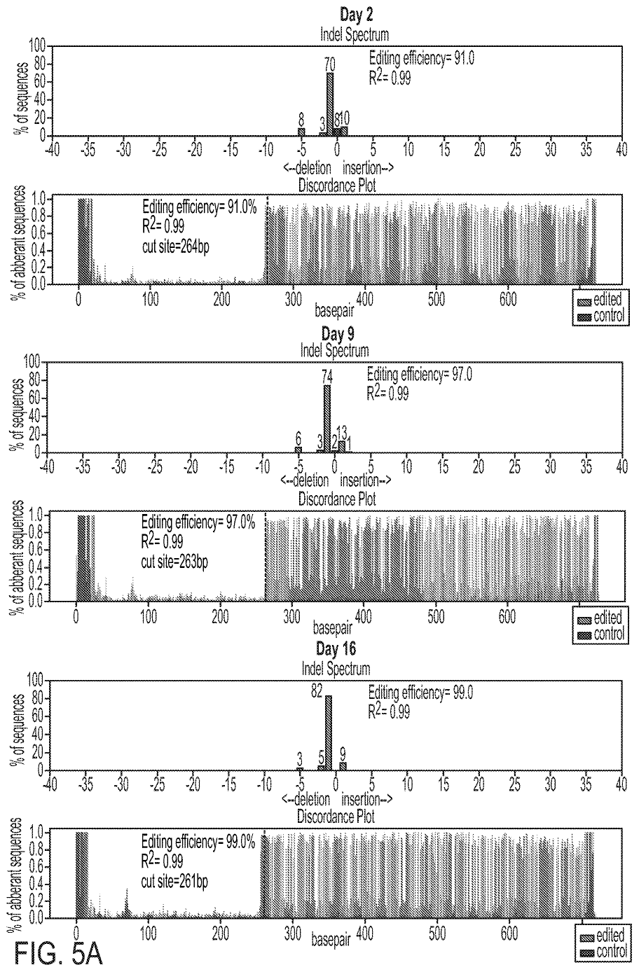

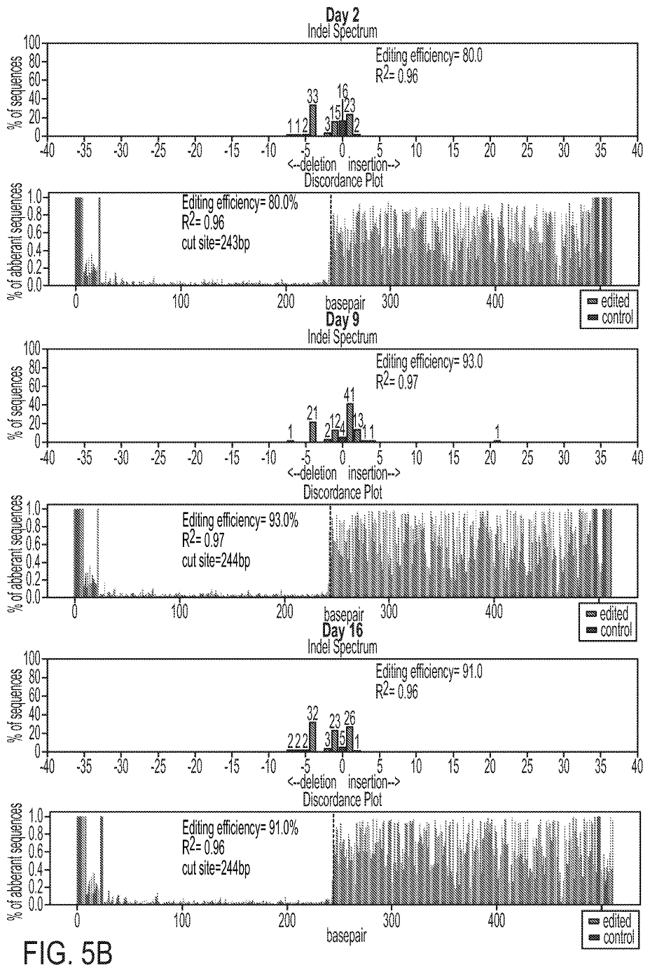

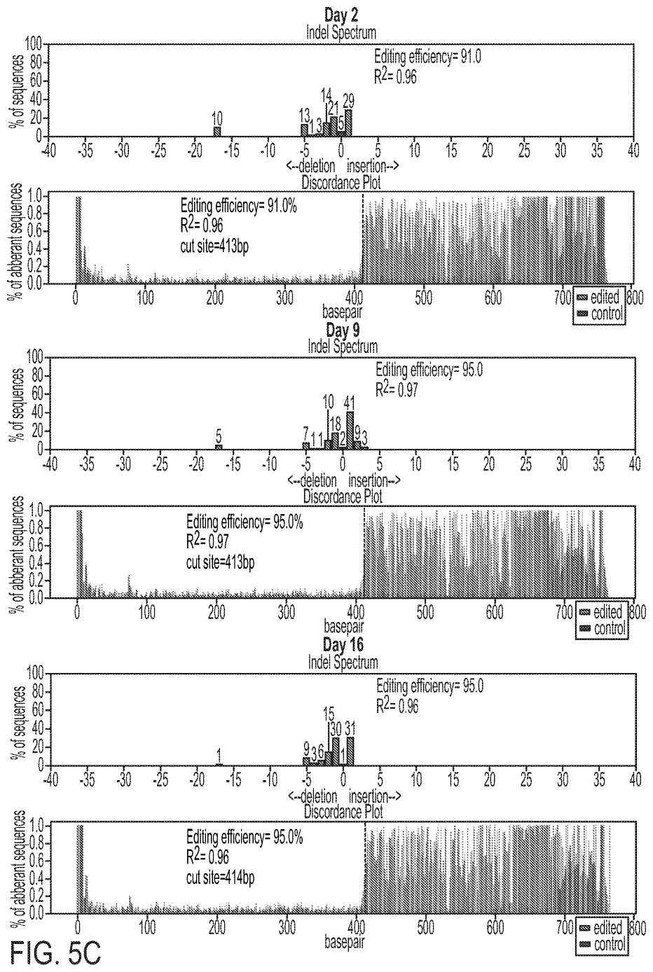

HL60 cells or human CD34+ cells were electroporated with Cas9 protein and indicated EMR2-targeting gRNAs, as described above. The percentage editing was determined by % INDEL as assessed by TIDE analysis. Editing efficiency was determined by flow cytometric analysis. See, FIGS. 2A-2F, 4B, 5A-5C. As shown in FIGS. 2C-2F, editing EMR2 with guide EMR2-11 was found to result in premature stop codons, either through the deletion of 4 nucleotides (−4) or addition of 1 nucleotide (+1).

### Expression and Flow Cytometry Analysis

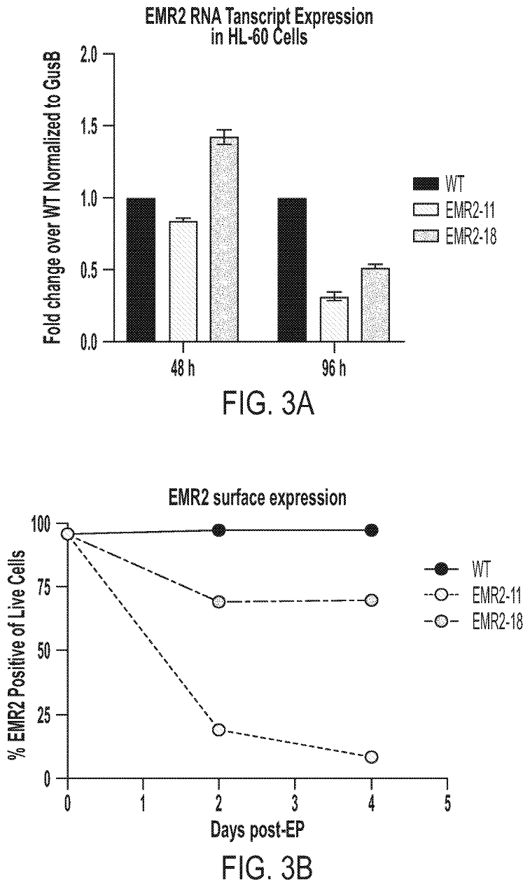

The EMR2 gRNA-edited cells were also evaluated for expression of EMR2 RNA transcripts and surface expression of EMR2 protein, for example by flow cytometry analysis (FACS). To assess surface expression, live CD34+ HSCs were stained for EMR2 using an anti-EMR2 antibody and analyzed by flow cytometry. Cells in which the EMR2 gene have been genetically modified showed a reduction in EMR2 expression as detected by FACS. See, FIGS. 3A and 3B.

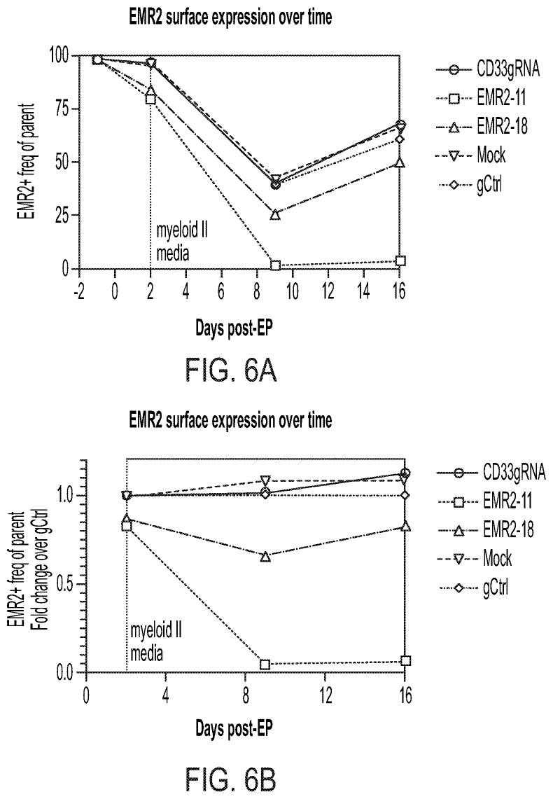

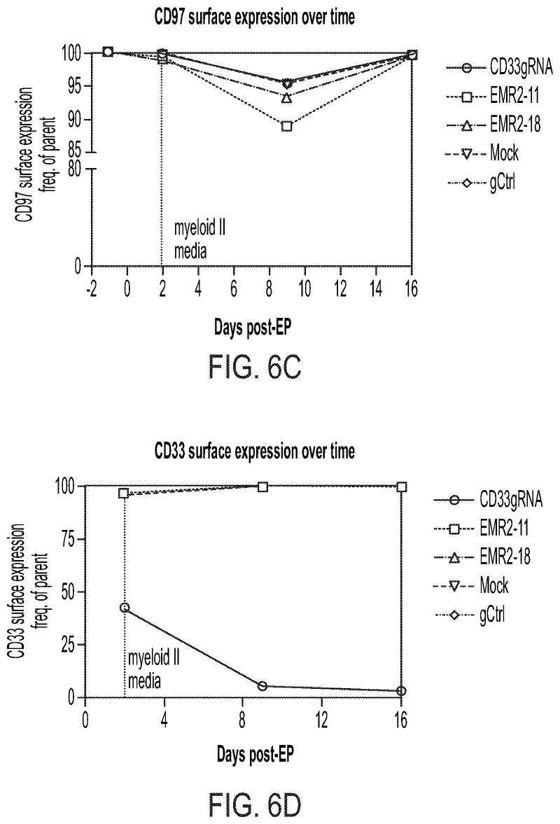

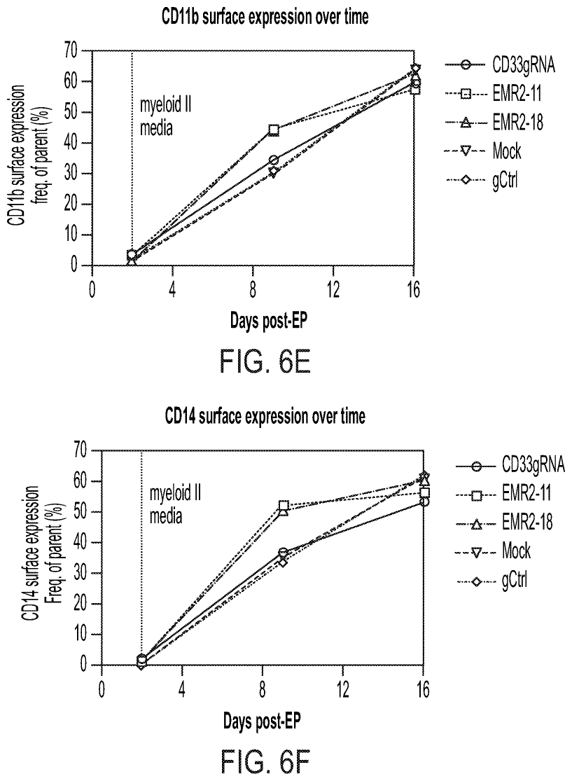

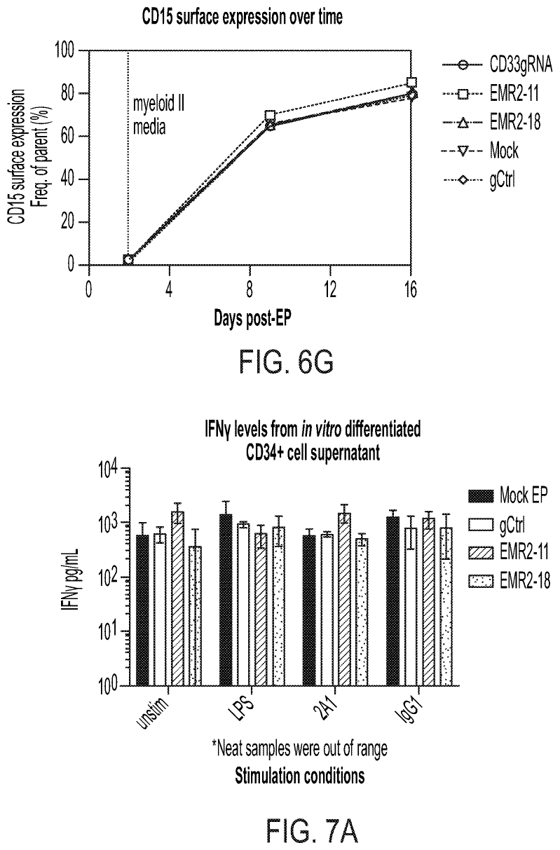

The EMR2 gRNA-edited cells were also evaluated for surface expression of EMR2 protein over time while cultured, which demonstrated a significant and sustained reduced in EMR2 expression. See, FIGS. 6A and 6B.

Surface expression of CD97 and CD33 as well as several myeloid markers (e.g., CD11b, CD14, CD15) were also evaluated over time. These markers were not significantly affected by editing of EMR2. See, FIGS. 6C-6G.

### Cytokine Levels

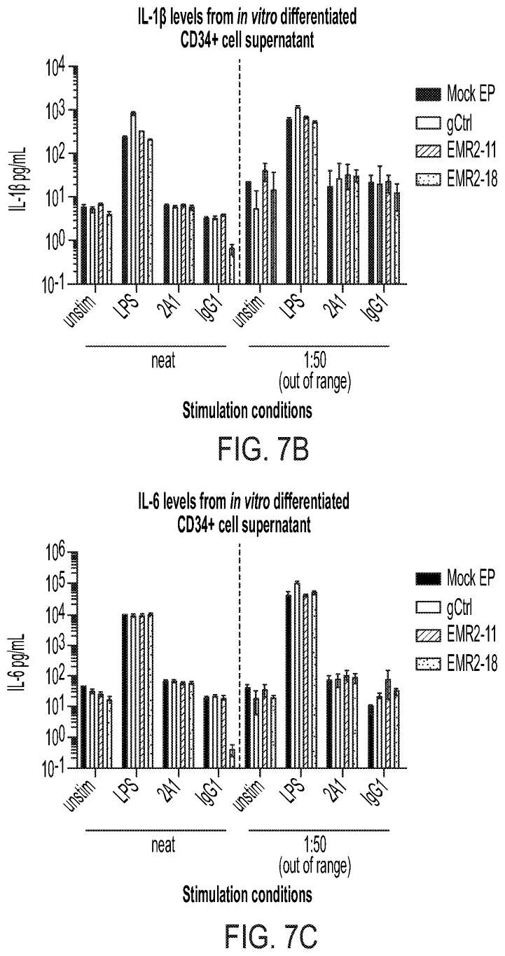

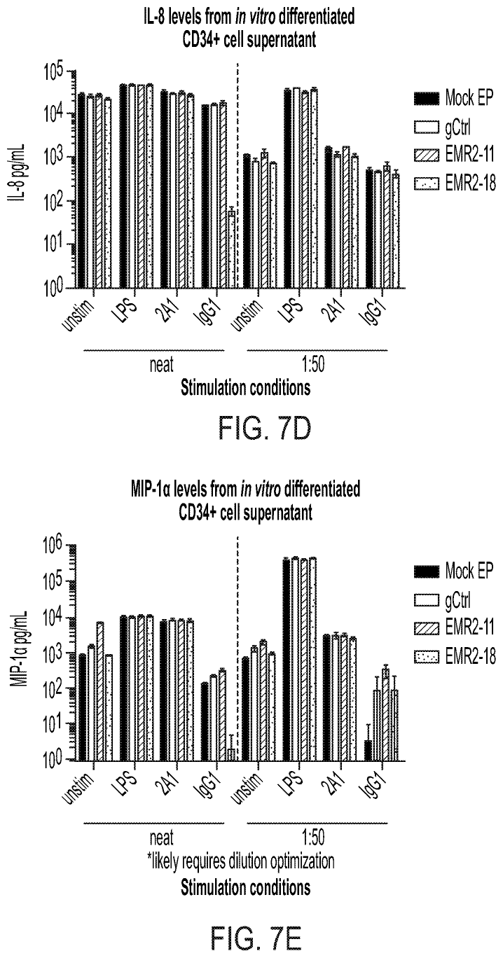

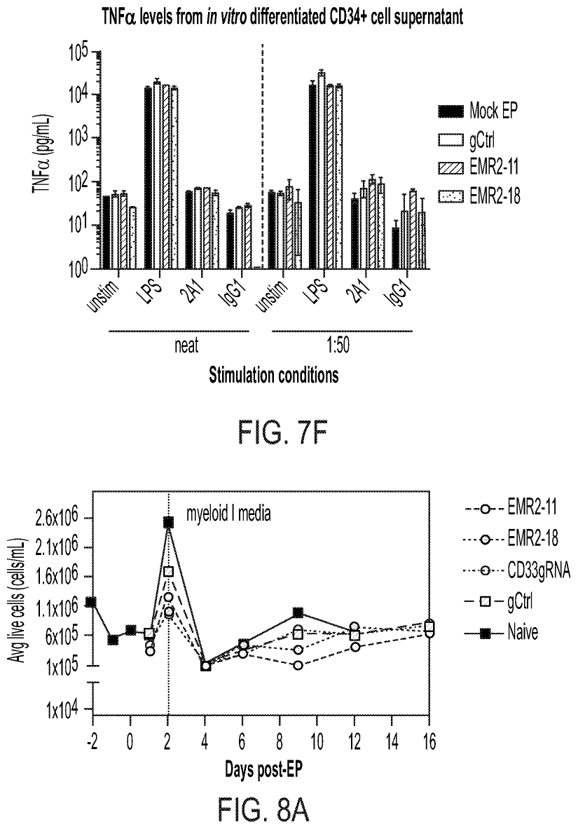

Cytokine production was assessed for in vitro differentiated CD34+ cells following stimulation with lipopolysaccharide (LPS), an anti-EMR2 antibody (2A1), or IgG1. See, FIGS. 7A-7F. As expected, LPS simulation did result in upregulation of pro-inflammatory cytokines as compared to unstimulated cells. No major shift in cytokine production (i.e., IFN-7, IL1b, IL-6, IL-8, MIP-1α, TNFα) was observed, regardless of editing, suggesting that loss of EMR2 expression does not impact cytokine secretion. In addition, minimal, if any, upregulation in cytokines was observed upon stimulation with 2A1.

### Viability of Edited Cells

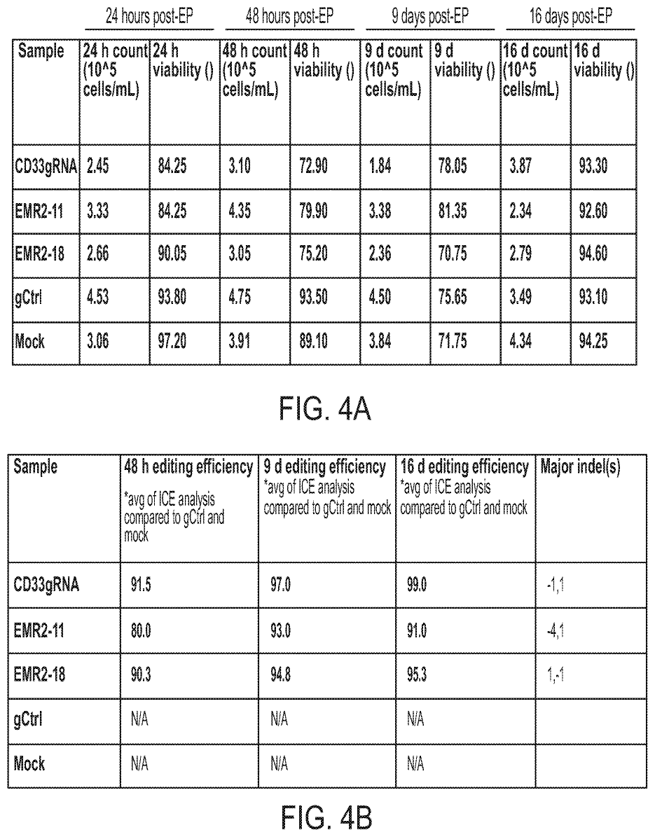

At various times post-ex vivo editing, e.g., 24 hours, 48 hours, 9 days, and 16 hours, the total number of cells and percentages of viable, edited EMR2KO cells and control cells were quantified. EMR2KO cells edited using the EMR2 gRNAs described herein were viable and remained viable over time following electroporation and gene editing. This was similar to what was observed in the control mock edited cells. See, FIG. 4A.

### Example 2: Generation and Evaluation of EMR2-Edited Cells

Genetically modified cells were produced using the exemplary gRNAs, EMR2-11 and EMR2-18, using cells obtained from a single donor and analyzed for in vitro differentiation. Genomic editing using a gRNA targeting CD33 (CD33gRNA) and a control gRNA (gCtrl) were also used. Following genomic editing, the cells were cultured in either granulocytic media (myeloid I media (Mye I)) or monocytic media (myeloid II media (Mye II)). The cell number and percent viability of cells were assessed at various times post-editing (post-electroporation).

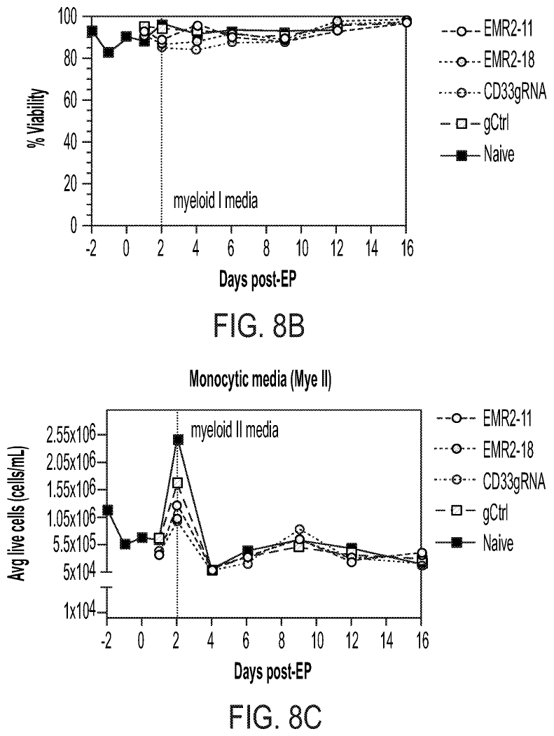

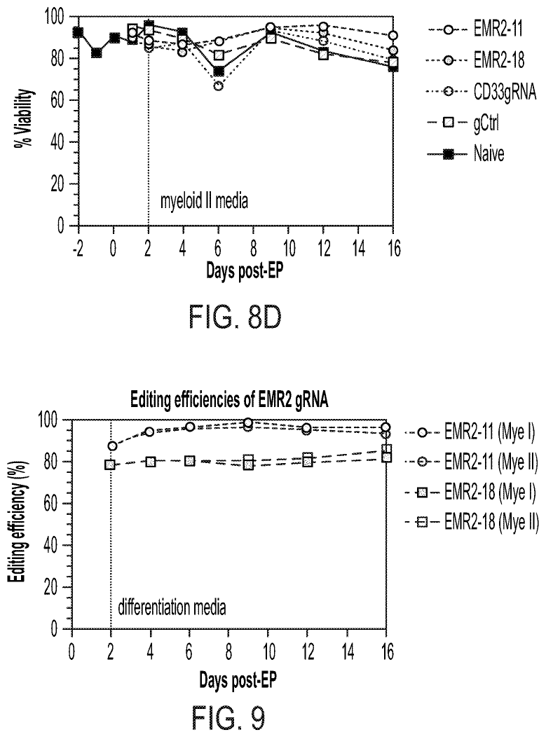

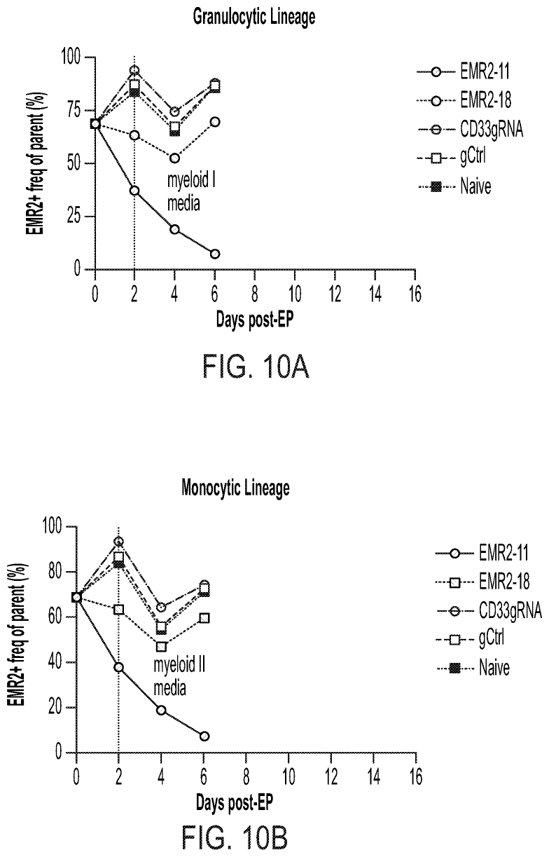

As shown in FIGS. 8A-8D, editing of EMR2 did not impact cell expansion or viability of granulocytic cells or monocytic cells, with more than 80% of EMR2-edited HSPCs retaining viability. The high level of EMR2 editing efficiency was also maintained throughout differentiation for the EMR2 gRNAs tested. See, FIG. 9. Editing with guide EMR2-11 was found to result in ablation of EMR2 surface expression by day 6 post-electroporation and persist through at least day 12 post electroporation. See, FIGS. 10A and 10B.

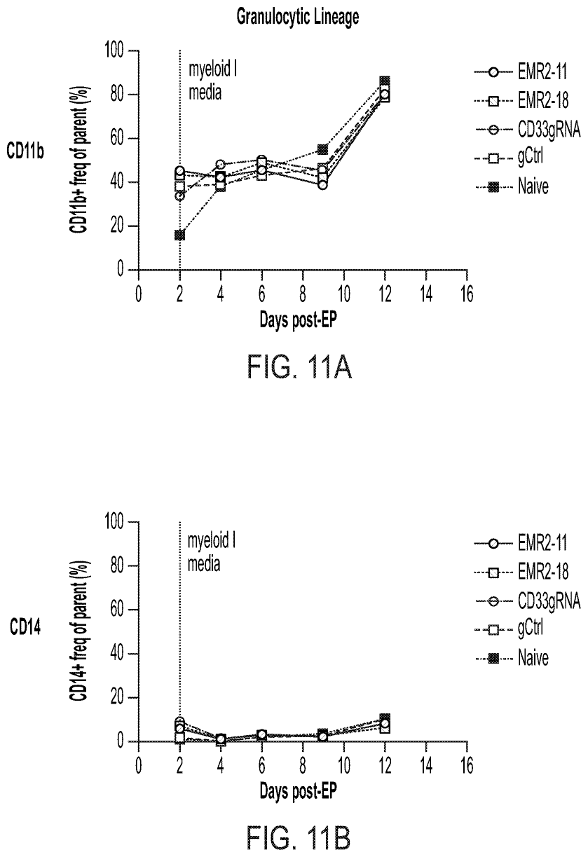

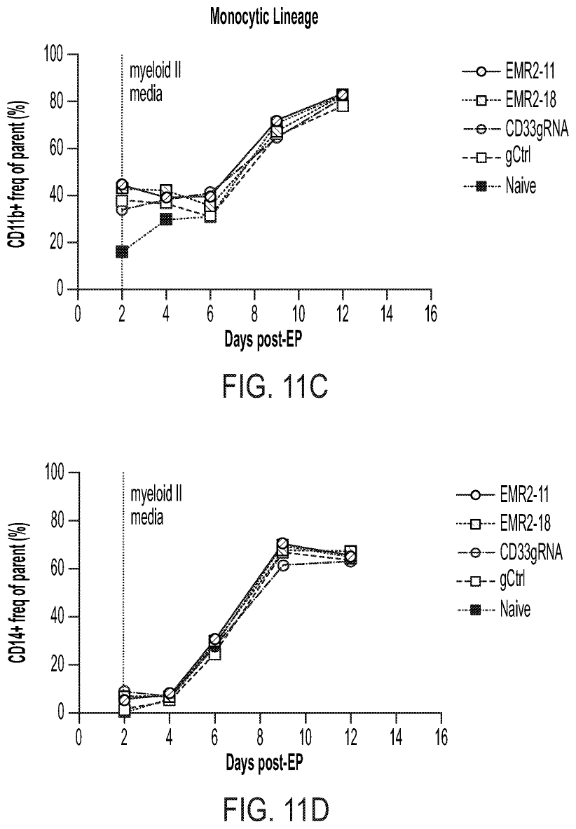

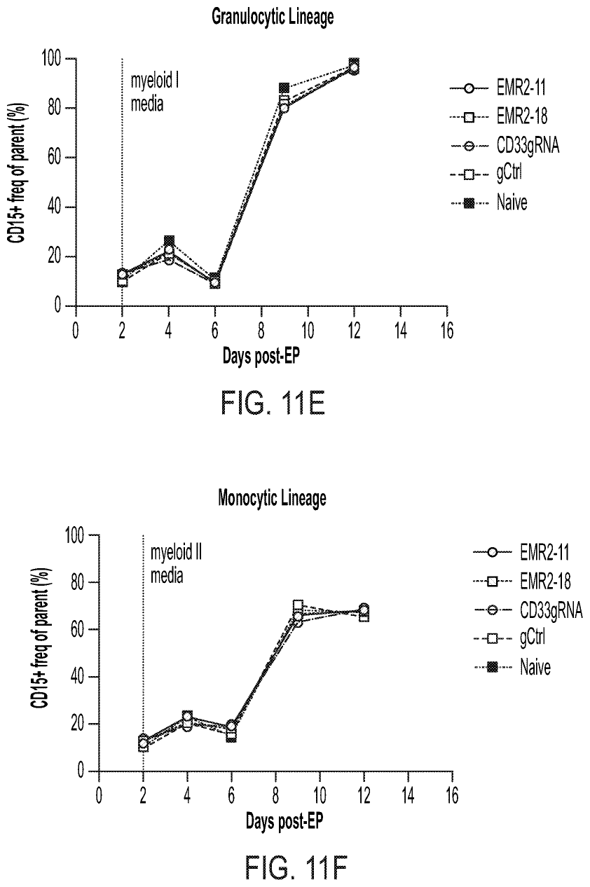

As observed in Example 1, editing of EMR2 did not impact expression of monocytic markers, CD11b or CD14, nor granulocytic-lineage marker CD15. See, FIGS. 11A-11F.

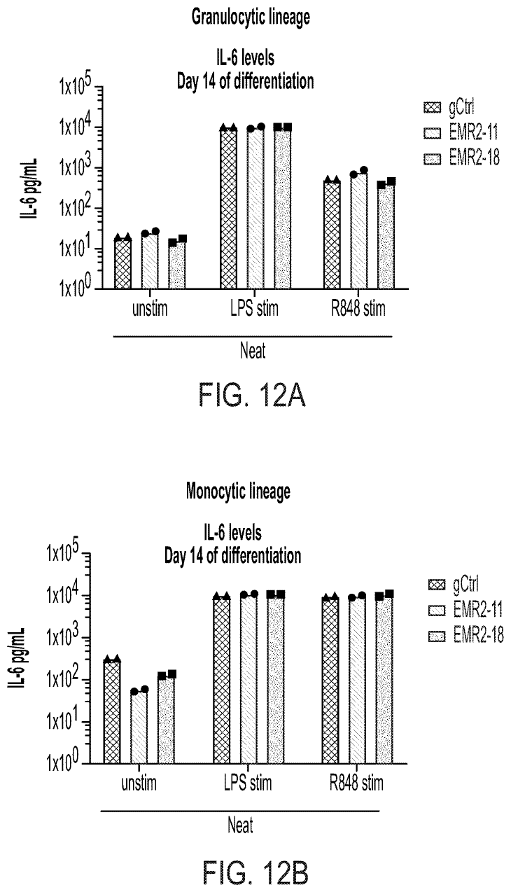

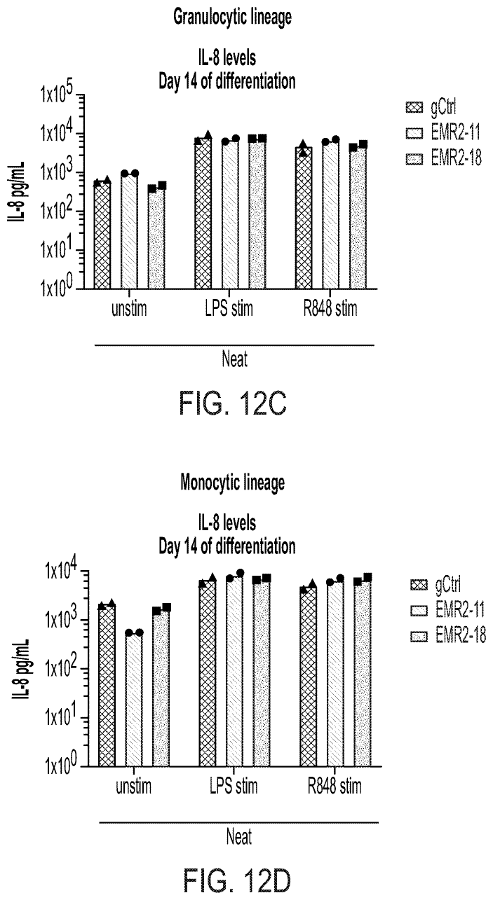

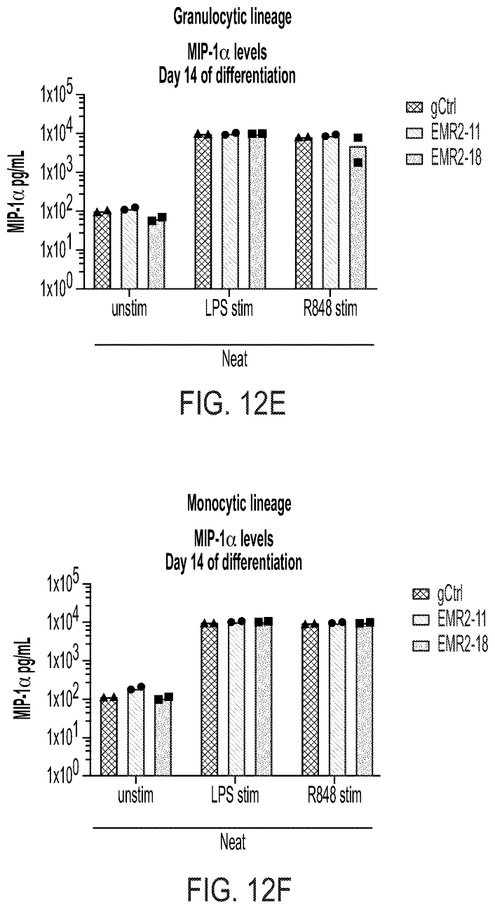

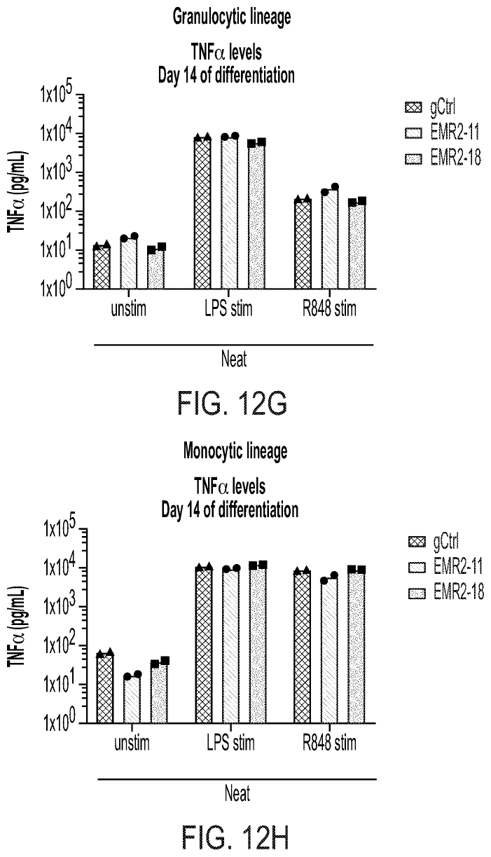

Production of cytokines was also assessed to determine whether EMR2 editing modulates cellular function. Cells were stimulated with LPS or R848, or unstimulated, and assessed for production of IL-6, IL-8, MIP-1α, and TNFα. As shown in FIGS. 12A-12H, EMR2 editing did not impact any of the cytokines measured, i.e., IL-6, IL-8, MIP-1α, and TNFα.

Cellular function of EMR2-edited cells, for example phagocytic ability, may also be evaluated.

### Example 3: Genetic Editing of EMR2 in Human Cells

Design of sgRNA Constructs

The target domains and gRNAs indicated in Tables 3-4 were designed by manual inspection for a PAM sequence for an applicable nuclease, e.g., a base editor nuclease, with close proximity to the target region and prioritized according to predicted specificity by minimizing potential off-target sites in the human genome with an online search algorithm (e.g., the Benchling algorithm, Doench et al 2016, Hsu et al 2013). All designed synthetic sgRNAs are produced with chemically modified nucleotides at the three terminal positions at both the 5′ and 3′ ends. Modified nucleotides contain 2′-O-methyl-3′-phosphorothioate (abbreviated as “ms”) and the ms-sgRNAs were HPLC-purified.

### Editing in Primary Human CD34+ HSCs

Screening of the EMR2 gRNAs is performed using the gRNAs shown in Tables 3 and 4, for example in HL60 cells or CD34+ cells. CD34+ HSCs derived from mobilized peripheral blood (mPB) are thawed according to manufacturer's instructions. To edit CD34+ HSCs, ˜1×106 HSCs are thawed and cultured in StemSpan SFEM medium supplemented with StemSpan CC110 cocktail (StemCell Technologies) for 24-48 h before electroporation with RNP. To electroporate HSCs, 1.5×105 cells are pelleted and resuspended in electroporation solution and mixed with ribonucleoprotein complexes containing the gRNA and nuclease are resuspended in electroporation solution and mixed with ribonucleoprotein complexes containing the gRNA and nuclease.

Exemplary base editor nucleases are provided below.

Amino acid sequence of exemplary cytidine
deaminase
(SEQ ID NO: 173)
MTSEKGPSTGDPTLRRRIESWEFDVFYDPRELRKETCLLYEIKWGMSRKI
WRSSGKNTTNHVEVNFIKKFTSERRFHSSISCSITWFLSWSPCWECSQAI
REFLSQHPGVTLVIYVARLFWHMDQRNRQGLRDLVNSGVTIQIMRASEYY
HCWRNFVNYPPGDEAHWPQYPPLWMMLYALELHCIILSLPPCLKISRRWQ
NHLAFFRLHLQNCHYQTIPPHILLATGLIHPSVTWRLKSGGSSGGSSGSE
TPGTSESATPESSGGSSGGSDKKYSIGLAIGTNSVGWAVITDEYKVPSKK
FKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQD
LTLLKALVRQQLPEKYKEIFFDQSKNGYAGYIDGGASQEEFYKFIKPILE
KMDGTEELLVKLNREDLLRKQRTFDNGSIPHQIHLGELHAILRRQEDFYP
FLKDNREKIEKILTFRIPYYVGPLARGNSRFAWMTRKSEETITPWNFEEV
VDKGASAQSFIERMINEDKNLPNEKVLPKHSLLYEYFTVYNELTKVKYVT
EGMRKPAFLSGEQKKAIVDLLFKTNRKVTVKQLKEDYFKKIECFDSVEIS
GVEDRFNASLGTYHDLLKIIKDKDELDNEENEDILEDIVLTLTLFEDREM
IEERLKTYAHLEDDKVMKQLKRRRYTGWGRLSRKLINGIRDKQSGKTILD
FLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERM
KRIEEGIKELGSQILKEHPVENTQLQNEKLYLYYLQNGRDMYVDQELDIN
RLSDYDVDHIVPQSFLKDDSIDNKVLTRSDKNRGKSDNVPSEEVVKKMKN
YWRQLLNAKLITQRKFDNLTKAERGGLSELDKAGFIKRQLVETRQITKHV
AQILDSRMNTKYDENDKLIREVKVITLKSKLVSDFRKDFQFYKVREINNY
HHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFA
TVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSK
YVNFLYLASHYEKLKGSPEDNEQKQLFVEQHKHYLDEIIEQISEFSKRVI
LADANLDKVLSAYNKHRDKPIREQAENIIHLFTLTNLGAPAAFKYFDTTI
DRKRYTSTKEVLDATLIHQSITGLYETRIDLSQLGGDSGGSGGSGGSTNL
SDIIEKETGKQLVIQESILMLPEEVEEVIGNKPESDILVHTAYDESTDEN
VMLLTSDAPEYKPWALVIQDSNGENKIKMLSGGSGGSGGSTNLSDIIEKE
TGKQLVIQESILMLPEEVEEVIGNKPESDILVHTAYDESTDENVMLLTSD
APEYKPWALVIQDSNGENKIKMLPKKKRKV
Nucleic acid sequence of exemplary cytidine
deaminase
(SEQ ID NO: 174)
atgacctctgagaagggccctagcacaggcgaccccaccctgcggcggag
aatcgagagctgggagttcgacgtgttctacgaccctagagaactgagaa
aggaaacctgcctgctgtacgagatcaagtggggcatgagcagaaagatc
tggcggagctctggcaagaacaccaccaaccacgtggaagtgaatttcat
caagaagttcaccagcgagagaaggttccacagcagcatcagctgcagca
tcacctggttcctgagctggtccccttgctgggaatgcagccaggccatc
agagagttcctgagccaacaccccggagtgacactggtgatctacgtggc
cagactgttctggcacatggaccagagaaacagacagggcctgagagatc
tggtcaacagcggcgtgactatccagatcatgcgggccagcgagtactac
cactgttggcggaacttcgtgaactacccccccggcgatgaggcccactg
gcctcagtaccctcctctgtggatgatgctgtacgccctggaactgcact
gcatcatcctgtctctgcctccatgtctgaagatctctagaagatggcag
aaccacctggccttcttcagactgcacctgcagaattgccactaccagac
catccccccccacatcctgctggctacaggcctgatccacccttctgtga
cctggagacttaagagcggaggatctagcggcggctctagcggatctgag
acacctggcacaagcgagtctgccacacctgagagtagcggcggatcttc
tggtggctctgacaagaagtacagcatcggcctggccatcggcaccaact
ctgtgggctgggccgtgatcaccgacgagtacaaggtgcccagcaagaaa
ttcaaggtgctgggcaacaccgaccggcacagcatcaagaagaacctgat
cggagccctgctgttcgacagcggcgaaacagccgaggccacccggctga
agagaaccgccagaagaagatacaccagacggaagaaccggatctgctat
ctgcaagagatcttcagcaacgagatggccaaggtggacgacagcttctt
ccacagactggaagagtccttcctggtggaagaggataagaagcacgagc
ggcaccccatcttcggcaacatcgtggacgaggtggcctaccacgagaag
taccccaccatctaccacctgagaaagaaactggtggacagcaccgacaa
ggccgacctgcggctgatctatctggccctggcccacatgatcaagttcc
ggggccacttcctgatcgagggcgacctgaaccccgacaacagcgacgtg
gacaagctgttcatccagctggtgcagacctacaaccagctgttcgagga
aaaccccatcaacgccagcggcgtggacgccaaggccatcctgtctgcca
gactgagcaagagcagacggctggaaaatctgatcgcccagctgcccggc
gagaagaagaatggcctgttcggaaacctgattgccctgagcctgggcct
gacccccaacttcaagagcaacttcgacctggccgaggatgccaaactgc
agctgagcaaggacacctacgacgacgacctggacaacctgctggcccag
atcggcgaccagtacgccgacctgtttctggccgccaagaacctgtccga
cgccatcctgctgagcgacatcctgagagtgaacaccgagatcaccaagg
cccccctgagcgcctctatgatcaagagatacgacgagcaccaccaggac
ctgaccctgctgaaagctctcgtgcggcagcagctgcctgagaagtacaa
agagattttcttcgaccagagcaagaacggctacgccggctacattgacg
gcggagccagccaggaagagttctacaagttcatcaagcccatcctggaa
aagatggacggcaccgaggaactgctcgtgaagctgaacagagaggacct
gctgcggaagcagcggaccttcgacaacggcagcatcccccaccagatcc
acctgggagagctgcacgccattctgcggcggcaggaagatttttaccca
ttcctgaaggacaaccgggaaaagatcgagaagatcctgaccttccgcat
cccctactacgtgggccctctggccaggggaaacagcagattcgcctgga
tgaccagaaagagcgaggaaaccatcaccccctggaacttcgaggaagtg
gtggacaagggcgcttccgcccagagcttcatcgagcggatgaccaactt
cgataagaacctgcccaacgagaaggtgctgcccaagcacagcctgctgt
acgagtacttcaccgtgtataacgagctgaccaaagtgaaatacgtgacc
gagggaatgagaaagcccgccttcctgagcggcgagcagaaaaaggccat
cgtggacctgctgttcaagaccaaccggaaagtgaccgtgaagcagctga
aagaggactacttcaagaaaatcgagtgcttcgactccgtggaaatctcc
ggcgtggaagatcggttcaacgcctccctgggcacataccacgatctgct
gaaaattatcaaggacaaggacttcctggacaatgaggaaaacgaggaca
ttctggaagatatcgtgctgaccctgacactgtttgaggacagagagatg
atcgaggaacggctgaaaacctatgcccacctgttcgacgacaaagtgat
gaagcagctgaagcggcggagatacaccggctggggcaggctgagccgga
agctgatcaacggcatccgggacaagcagtccggcaagacaatcctggat
ttcctgaagtccgacggcttcgccaacagaaacttcatgcagctgatcca
cgacgacagcctgacctttaaagaggacatccagaaagcccaggtgtccg
gccagggcgatagcctgcacgagcacattgccaatctggccggcagcccc
gccattaagaagggcatcctgcagacagtgaaggtggtggacgagctcgt
gaaagtgatgggccggcacaagcccgagaacatcgtgatcgaaatggcca
gagagaaccagaccacccagaagggacagaagaacagccgcgagagaatg
aagcggatcgaagagggcatcaaagagctgggcagccagatcctgaaaga
acaccccgtggaaaacacccagctgcagaacgagaagctgtacctgtact
acctgcagaatggggggatatgtacgtggaccaggaactggacatcaacc
ggctgtccgactacgatgtggaccatatcgtgcctcagagctttctgaag
gacgactccatcgacaacaaggtgctgaccagaagcgacaagaaccgggg
caagagcgacaacgtgccctccgaagaggtcgtgaagaagatgaagaact
actggcggcagctgctgaacgccaagctgattacccagagaaagttcgac
aatctgaccaaggccgagagaggcggcctgagcgaactggataaggccgg
cttcatcaagagacagctggtggaaacccggcagatcacaaagcacgtgg
cacagatcctggactcccggatgaacactaagtacgacgagaatgacaag
ctgatccgggaagtgaaagtgatcaccctgaagtccaagctggtgtccga
tttccggaaggatttccagttttacaaagtgcgcgagatcaacaactacc
accacgcccacgacgcctacctgaacgccgtcgtgggaaccgccctgatc
aaaaagtaccctaagctggaaagcgagttcgtgtacggcgactacaaggt
gtacgacgtgcggaagatgatcgccaagagcgagcaggaaatcggcaagg
ctaccgccaagtacttcttctacagcaacatcatgaactttttcaagacc
gagattaccctggccaacggcgagatccggaagcggcctctgatcgagac
aaacggcgaaaccggggagatcgtgtgggataagggccgggattttgcca
ccgtgcggaaagtgctgagcatgccccaagtgaatatcgtgaaaaagacc
gaggtgcagacaggcggcttcagcaaagagtctatcctgcccaagaggaa
cagcgataagctgatcgccagaaagaaggactgggaccctaagaagtacg
gcggcttcgacagccccaccgtggcctattctgtgctggtggtggccaaa
gtggaaaagggcaagtccaagaaactgaagagtgtgaaagagctgctggg
gatcaccatcatggaaagaagcagcttcgagaagaatcccatcgactttc
tggaagccaagggctacaaagaagtgaaaaaggacctgatcatcaagctg
cctaagtactccctgttcgagctggaaaacggccggaagagaatgctggc
ctctgccggcgaactgcagaagggaaacgaactggccctgccctccaaat
atgtgaacttcctgtacctggccagccactatgagaagctgaagggctcc
cccgaggataatgagcagaaacagctgtttgtggaacagcacaagcacta
cctggacgagatcatcgagcagatcagcgagttctccaagagagtgatcc
tggccgacgctaatctggacaaagtgctgtccgcctacaacaagcaccgg
gataagcccatcagagagcaggccgagaatatcatccacctgtttaccct
gaccaatctgggagcccctgccgccttcaagtactttgacaccaccatcg
accggaagaggtacaccagcaccaaagaggtgctggacgccaccctgatc
caccagagcatcaccggcctgtacgagacacggatcgacctgtctcagct
gggaggtgactctggtggaagcggaggatctggcggcagcaccaatctga
gcgacatcatcgagaaagagacaggcaagcagctggtcatccaagagtcc
atcctgatgctgcctgaagaggtggaagaagtgatcggcaacaagcccga
gtccgacatcctggtgcacaccgcctacgatgagagcaccgacgagaacg
tgatgctgctgacctctgacgcccctgagtacaagccttgggctctcgtg
atccaggacagcaacggcgagaacaagatcaagatgctgagcggcggctc
tggtggctctggcggatctacaaacctgtccgatattattgagaaagaaa
ccgggaaacagctcgtgattcaagagtctattctcatgctcccggaagaa
gtcgaggaagtcattggaaacaagcctgagagcgatattctggtccatac
agcctacgacgagtctaccgatgagaatgtcatgctcctcaccagcgacg
ctcccgagtataagccatgggcacttgtcattcaggactccaatggggaa
aacaaaatcaaaatgctcccaaagaaaaaacgcaaggtgtag
Amino acid sequence of exemplary adenosine
deaminase
(SEQ ID NO: 175)
MSEVEFSHEYWMRHALTLAKRARDEREVPVGAVLVLNNRVIGEGWNRAIG
LHDPTAHAEIMALRQGGLVMQNYRLYDATLYSTFEPCVMCAGAMIHSRIG
RVVFGVRNAKTGAAGSLMDVLHHPGMNHRVEITEGILADECAALLCRFFR
MPRRVFNAQKKAQSSTDSGGSSGGSSGSETPGTSESATPESSGGSSGGSD
KKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLL
FKTNRKVTVKQLKEDYFKKIECFDSVEISGVEDRENASLGTYHDLLKIIK
DKDELDNEENEDILEDIVLTLTLFEDREMIEERLKTYAHLFDDKVMKQLK
RRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMG
RHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYS
LFELENGRKRMLASAGELQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSI
TGLYETRIDLSQLGGDEGADKRTADGSEFESPKKKRKV
Nucleic acid sequence of exemplary adenosine
deaminase
(SEQ ID NO: 176)
atgtccgaagtcgagttttcccatgagtactggatgagacacgcattgac
tctcgcaaagagggctcgagatgaacgcgaggtgcccgtgggggcagtac
tcgtgctcaacaatcgcgtaatcggcgaaggttggaatagggcaatcgga
ctccacgaccccactgcacatgcggaaatcatggcccttcgacagggagg
gcttgtgatgcagaattatcgactttatgatgcgacgctgtactcgacgt
ttgaaccttgcgtaatgtgcgcgggagctatgattcactcccgcattgga
cgagttgtattcggtgttcgcaacgccaagacgggtgccgcaggttcact
gatggacgtgctgcatcatccaggcatgaaccaccgggtagaaatcacag
aaggcatattggcggacgaatgtgcggcgctgttgtgtcgtttttttcgc
atgcccaggcgggtctttaacgcccagaaaaaagcacaatcctctactga
ctctggtggttcttctggtggttctagcggcagcgagactcccgggacct
cagagtccgccacacccgaaagttctggtggttcttctggtggttctgac
aagaagtacagcatcggcctggccatcggcaccaactctgtgggctgggc
cgtgatcaccgacgagtacaaggtgcccagcaagaaattcaaggtgctgg
gcaacaccgaccggcacagcatcaagaagaacctgatcggagccctgctg
ttcgacagcggcgaaacagccgaggccacccggctgaagagaaccgccag
aagaagatacaccagacggaagaaccggatctgctatctgcaagagatct
tcagcaacgagatggccaaggtggacgacagcttcttccacagactggaa
gagtccttcctggtggaagaggataagaagcacgagcggcaccccatctt
cggcaacatcgtggacgaggtggcctaccacgagaagtaccccaccatct
accacctgagaaagaaactggtggacagcaccgacaaggccgacctgcgg
ctgatctatctggccctggcccacatgatcaagttccggggccacttcct
gatcgagggcgacctgaaccccgacaacagcgacgtggacaagctgttca
tccagctggtgcagacctacaaccagctgttcgaggaaaaccccatcaac
gccagcggcgtggacgccaaggccatcctgtctgccagactgagcaagag
cagacggctggaaaatctgatcgcccagctgcccggcgagaagaagaatg
gcctgttcggaaacctgattgccctgagcctgggcctgacccccaacttc
aagagcaacttcgacctggccgaggatgccaaactgcagctgagcaagga
cacctacgacgacgacctggacaacctgctggcccagatcggcgaccagt
acgccgacctgtttctggccgccaagaacctgtccgacgccatcctgctg
agcgacatcctgagagtgaacaccgagatcaccaaggcccccctgagcgc
ctctatgatcaagagatacgacgagcaccaccaggacctgaccctgctga
aagctctcgtgcggcagcagctgcctgagaagtacaaagagattttcttc
gaccagagcaagaacggctacgccggctacattgacggcggagccagcca
ggaagagttctacaagttcatcaagcccatcctggaaaagatggacggca
ccgaggaactgctcgtgaagctgaacagagaggacctgctgcggaagcag
cggaccttcgacaacggcagcatcccccaccagatccacctgggagagct
gcacgccattctgcggcggcaggaagatttttacccattcctgaaggaca
accgggaaaagatcgagaagatcctgaccttccgcatcccctactacgtg
ggccctctggccaggggaaacagcagattcgcctggatgaccagaaagag
cgaggaaaccatcaccccctggaacttcgaggaagtggtggacaagggcg
cttccgcccagagcttcatcgagcggatgaccaacttcgataagaacctg
cccaacgagaaggtgctgcccaagcacagcctgctgtacgagtacttcac
cgtgtataacgagctgaccaaagtgaaatacgtgaccgagggaatgagaa
agcccgccttcctgagcggcgagcagaaaaaggccatcgtggacctgctg
ttcaagaccaaccggaaagtgaccgtgaagcagctgaaagaggactactt
caagaaaatcgagtgcttcgactccgtggaaatctccggcgtggaagatc
ggttcaacgcctccctgggcacataccacgatctgctgaaaattatcaag
gacaaggacttcctggacaatgaggaaaacgaggacattctggaagatat
cgtgctgaccctgacactgtttgaggacagagagatgatcgaggaacggc
tgaaaacctatgcccacctgttcgacgacaaagtgatgaagcagctgaag
cggcggagatacaccggctggggcaggctgagccggaagctgatcaacgg
catccgggacaagcagtccggcaagacaatcctggatttcctgaagtccg
acggcttcgccaacagaaacttcatgcagctgatccacgacgacagcctg
acctttaaagaggacatccagaaagcccaggtgtccggccagggcgatag
cctgcacgagcacattgccaatctggccggcagccccgccattaagaagg
gcatcctgcagacagtgaaggtggtggacgagctcgtgaaagtgatgggc
cggcacaagcccgagaacatcgtgatcgaaatggccagagagaaccagac
cacccagaagggacagaagaacagccgcgagagaatgaagcggatcgaag
agggcatcaaagagctgggcagccagatcctgaaagaacaccccgtggaa
aacacccagctgcagaacgagaagctgtacctgtactacctgcagaatgg
ggggatatgtacgtggaccaggaactggacatcaaccggctgtccgacta
cgatgtggaccatatcgtgcctcagagctttctgaaggacgactccatcg
acaacaaggtgctgaccagaagcgacaagaaccggggcaagagcgacaac
gtgccctccgaagaggtcgtgaagaagatgaagaactactggcggcagct
gctgaacgccaagctgattacccagagaaagttcgacaatctgaccaagg
ccgagagaggcggcctgagcgaactggataaggccggcttcatcaagaga
cagctggtggaaacccggcagatcacaaagcacgtggcacagatcctgga
ctcccggatgaacactaagtacgacgagaatgacaagctgatccgggaag
tgaaagtgatcaccctgaagtccaagctggtgtccgatttccggaaggat
ttccagttttacaaagtgcgcgagatcaacaactaccaccacgcccacga
cgcctacctgaacgccgtcgtgggaaccgccctgatcaaaaagtacccta
agctggaaagcgagttcgtgtacggcgactacaaggtgtacgacgtgcgg
aagatgatcgccaagagcgagcaggaaatcggcaaggctaccgccaagta
cttcttctacagcaacatcatgaactttttcaagaccgagattaccctgg
ccaacggcgagatccggaagcggcctctgatcgagacaaacggcgaaacc
ggggagatcgtgtgggataagggccgggattttgccaccgtgcggaaagt
gctgagcatgccccaagtgaatatcgtgaaaaagaccgaggtgcagacag
gcggcttcagcaaagagtctatcctgcccaagaggaacagcgataagctg
atcgccagaaagaaggactgggaccctaagaagtacggcggcttcgacag
ccccaccgtggcctattctgtgctggtggtggccaaagtggaaaagggca
agtccaagaaactgaagagtgtgaaagagctgctggggatcaccatcatg
gaaagaagcagcttcgagaagaatcccatcgactttctggaagccaaggg
ctacaaagaagtgaaaaaggacctgatcatcaagctgcctaagtactccc
tgttcgagctggaaaacggccggaagagaatgctggcctctgccggcgaa
ctgcagaagggaaacgaactggccctgccctccaaatatgtgaacttcct
gtacctggccagccactatgagaagctgaagggctcccccgaggataatg
agcagaaacagctgtttgtggaacagcacaagcactacctggacgagatc
atcgagcagatcagcgagttctccaagagagtgatcctggccgacgctaa
tctggacaaagtgctgtccgcctacaacaagcaccgggataagcccatca
gagagcaggccgagaatatcatccacctgtttaccctgaccaatctggga
gcccctgccgccttcaagtactttgacaccaccatcgaccggaagaggta
caccagcaccaaagaggtgctggacgccaccctgatccaccagagcatca
ccggcctgtacgagacacggatcgacctgtctcagctgggaggtgacgag
ggagctgataagcgcaccgccgatggttccgagttcgaaagccccaagaa
gaagaggaaagtctag

For all genomic analysis, DNA is harvested from cells, amplified with primers flanking the target region, purified and the allele modification frequencies were analyzed. Editing efficiency is determined by flow cytometric analysis.

### Expression and Flow Cytometry Analysis

The EMR2 gRNA-edited cells may also be evaluated for expression of EMR2 RNA transcripts and surface expression of EMR2 protein, for example by flow cytometry analysis FACS.

Expression of other surface markers, such as CD97 and CD33 as well as several myeloid markers (e.g., CD11b, CD14, CD15) may also be evaluated.

### Viability of Edited Cells

At various times post-ex vivo editing, e.g., 24 hours, 48 hours, 9 days, and 16 hours, the total number of cells and percentages of viable, EMR2 edited cells and control cells are quantified.

### Example 4: CAR-T Cytotoxicity Assay

Genetically modified cells produced using the gRNAs shown in Tables 1-8 may be evaluated for killing by EMR2-CAR T cells.

### CAR Constructs and Lentiviral Production

Second-generation CARs are constructed to target EMR2. An exemplary CAR construct consists of an extracellular scFv antigen-binding domain, using CD8α signal peptide, CD8α hinge and transmembrane regions, the 4-1BB costimulatory domain, and the CD3ξ signaling domain. The anti-EMR2 scFv sequence may be obtained from any anti-EMR2 antibody known in the art. CAR cDNA sequences for the target are sub-cloned into the multiple cloning site of the pCDH-EF1α-MCS-T2A-GFP expression vector, and lentivirus is generated following the manufacturer's protocol (System Biosciences). Lentivirus can be generated by transient transfection of 293TN cells (System Biosciences) using Lipofectamine 3000 (ThermoFisher). The exemplary CAR construct is generated by cloning the light and heavy chain of an anti-EMR2 antibody, to the CD8c hinge domain, the ICOS transmembrane domain, the ICOS signaling domain, the 4-1BB signaling domain and the CD3ξ signaling domain into the lentiviral plasmid pHIV-Zsgreen.

### CAR Transduction and Expansion

Human primary T cells are isolated from Leuko Pak (Stem Cell Technologies) by magnetic bead separation using anti-CD4 and anti-CD8 microbeads according to the manufacturer's protocol (Stem Cell Technologies). Purified CD4+ and CD8+ T cells are mixed 1:1 and activated using anti-CD3/CD28 coupled Dynabeads (Thermo Fisher) at a 1:1 bead to cell ratio. T cell culture media used is CTS Optimizer T cell expansion media supplemented with immune cell serum replacement, L-Glutamine and GlutaMAX (all purchased from Thermo Fisher) and 100 IU/mL of IL-2 (Peprotech). T cell transduction is performed 24 hours post activation by spinoculation in the presence of polybrene (Sigma). CAR-T cells are cultured for 9 days prior to cryopreservation. Prior to all experiments, T cells are thawed and rested at 37° C. for 4-6 hours.

### Flow Cytometry Based CAR-T Cytotoxicity Assay

The cytotoxicity of target cells is measured by comparing survival of target cells relative to the survival of negative control cells. For EMR2 cytotoxicity assays, wildtype and CRISPR/Cas9 edited cells of a EMR2-expressing AML cell line are used as target cells. Wildtype Raji cell lines (ATCC) are used as negative control for both experiments. Alternatively, CD34+ cells may be used as target cells and CD34+ cells deficient in EMR2 or having reduced expression of EMR2, or expressing a variant of EMR2, may be generated as described in Examples 1-3.

Target cells and negative control cells are stained with CellTrace Violet (CTV) and CFSE (Thermo Fisher), respectively, according to the manufacturer's instructions. After staining, target cells and negative control cells are mixed at 1:1.

Anti-EMR2 CAR-T cells are used as effector T cells. Non-transduced T cells (mock CAR-T) are used as control. The effector T cells are co-cultured with the target cell/negative control cell mixture at a 1:1 effector to target ratio in duplicate. A group of target cell/negative control cell mixture alone without effector T cells is included as control. Cells are incubated at 37° C. for 24 hours before flow cytometric analysis. Propidium iodide (ThermoFisher) is used as a viability dye. For the calculation of specific cell lysis, the fraction of live target cell to live negative control cell (termed target fraction) is used. Specific cell lysis is calculated as ((target fraction without effector cells−target fraction with effector cells)/(target fraction without effectors))×100%.

### Example 5: Effect of Anti-EMR2 Antibody Drug Conjugates on Engineered HSCs

Genetically modified cells produced using the gRNAs shown in Tables 1-8 may be evaluated for killing by antibody-drug conjugates, such as an anti-EMR2 antibody conjugated to an immunotoxin.

Frozen CD34+ HSPCs derived from mobilized peripheral blood are thawed and cultured for 72 h before electroporation with ribonucleoprotein comprising Cas9 and an sgRNA. Samples are electroporated with the following conditions:
i. Mock (nuclease only (e.g., Cas9), and
ii. KO sgRNA (such as any one of the EMR2 gRNAs shown in Tables 1-8). Cells are allowed to recover for 72 hours and genomic DNA is collected and analyzed. The percentage of EMR2-positive cells is assessed by flow cytometry to confirm that editing with the EMR2 gRNAs is effective in knocking out or reducing EMR2. The editing events in the HSCs result in a variety of indel sequences.

### Sensitivity of Cells Having EMR2 Deletion to Antibody-Drug Conjugates

To determine in vitro toxicity, cells are incubated with the antibody-drug conjugate in the culture media and the number of viable cells is quantified over time. Engineered cells that are deficient in EMR2 or have reduced EMR2 expression generated with the EMR2 gRNAs described herein are more resistant to antibody-drug conjugate treatment than cells expressing full length EMR2 (mock).

### (ii) Enrichment of EMR2-Modified Cells

To assay if EMR2-modified cells are enriched following treatment with the antibody-drug conjugate, CD34+ HSPCs are edited with 50% of standard nuclease (e.g., Cas9, Cpf1) to gRNA ratios. The bulk population of cells are analyzed prior to and after treatment with the antibody-drug conjugate. Following treatment with the antibody-drug conjugate, EMR2-modified cells are enriched so that the percentage of EMR2-deficient cells increased.

(iii) In Vitro Differentiation of CD34+ HSPCs

Cell populations are assessed for lymphoid differentiation prior to and after treatment with the antibody-drug conjugate at various days post differentiation. Engineered EMR2 knockout cells generated with the EMR2 gRNAs described herein may show increased expression of lymphoid differentiation markers, whereas cells expressing full length EMR2 (mock) may not differentiate.

### Example 6: Evaluation of the Persistence of EMR2KO CD34+ Cells In Vivo

### Editing in Mobilized Peripheral Blood CD34+ HSCs (mPB CD34+ HSPCs)

gRNAs were designed as described in Examples 1 and 3. mPB CD34+ HSPCs were thawed according to manufacturer's instructions. These cells are then edited via a nuclease (e.g., CRISPR/Cas9) as described in Examples 1-3 using the EMR2-targeting gRNAs described herein, as well as a non-EMR2 targeting control gRNA (gCtrl) that is designed not to target any region in the human or mouse genomes.

At 4, 24, and 48 hours post-ex vivo editing, the percentages of viable, edited EMR2KO cells and control cells are quantified using flow cytometry and the 7AAD viability dye. High levels of EMR2KO cells edited using the EMR2 gRNAs described herein may be viable and remain viable over time following electroporation and gene editing, comparable to what is observed in the control cells edited with the non-EMR2 targeting control gRNA, gCtrl.

Additionally, at 48 hours post-ex vivo editing, the genomic DNA is harvested from cells, PCR amplified with primers flanking the target region, purified, and analyzed by TIDE, in order to determine the percentage editing as assessed by INDEL (insertion/deletion), as described in Example 1.

Following TIDE analysis, the percentage of long term-HSCs (LT-HSCs) following editing with the EMR2 gRNAs described herein are quantified by flow cytometry. The percentages of LT-HSCs following editing with the specified EMR2 gRNAs is assessed. This assay may be performed, for example, at the time of cryopreservation of the edited cells, prior to injection into mice for investigation of persistence of EMR2KO cells in vivo. The edited cells are cryopreserved in CryoStor® CS10 media (Stem Cell Technology) at 5×10⁶ cells/mL, in a 1 mL volume of media per vial.

### Investigating Engraftment Efficiency and Persistence of EMR2KO mPB CD34+ HSPCs In Vivo

Female NSG mice (JAX) that are 6 to 8 weeks of age, are allowed to acclimate for 2-7 days. Following acclimation, mice are irradiated using 175 cGy whole body irradiation by X-ray irradiator. This is regarded as day 0 of the investigation. At 4-10 hours, following irradiation, the mice are engrafted with the EMR2KO cells generated during any of the EMR2 gRNAs described herein or control cells edited with gCtrl. The cryopreserved cells are thawed and counted using a BioRad TC-20 automated cell counter. The number of viable cells is quantified in the thawed vials, which is used to prepare the total number of cells for engraftment in the mice. Mice are given a single intravenous injection of 1×10⁶ edited cells in a 100 μL volume. Body weight and clinical observations are recorded once weekly for each mouse in the four groups.

At weeks 8 and 12 following engraftment, 50 μL of blood is collected from each mouse by retroorbital bleed for analysis by flow cytometry. At week 16, following engraftment, mice are sacrificed, and blood, spleens, and bone marrow are collected for analysis by flow cytometry. Bone marrow is isolated from the femur and the tibia. Bone marrow from the femur is also used for on-target editing analysis. Flow cytometry is performed using the FACSCanto™ 10 color and BDFACSDiva™ software. Cells are generally first sorted by viability using the 7AAD viability dye (live/dead analysis), then live cells are gated by expression of human CD45 (hCD45) but not mouse CD45 (mCD45). The cells that are hCD45+ are then further gated for the expression of human CD19 (hCD19) (lymphoid cells, specifically B cells). Cells expressing human CD45 (hCD45) are also gated and analyzed for the presence of for various cellular markers of the myeloid lineage.

Numbers of cells expressing each of the analyzed markers that are comparable across all mice regardless of which edited cells they are engrafted with indicates successful engraftment of EMR2KO cells edited with the EMR2 gRNAs in the blood of mice.

At weeks 8, 12, and 16 following engraftment, the percentage of nucleated blood cells that are hCD45+ is quantified in the groups of mice (n=15 mice/group) that received control cells edited with the control gRNA (gCtrl), or the EMR2KO cells. This is quantified by dividing the hCD45+ absolute cell count by the mouse CD45+(mCD45) absolute cell count.

The percentage of hEMR2+ cells in the blood is also quantified at week 8 following engraftment in the control and EMR2KO mouse groups. Mice engrafted with the EMR2KO cells (edited with any of the EMR2 gRNAs described herein) are expected to have significantly lower levels of hEMR2+ cells compared to the mice engrafted with control cells at weeks 8, 12, and 16.

Next, the percentages of particular populations of differentiated cells, such as CD19+ lymphoid cells, hCD14+ monocytes, and hCD11b+ granulocytes/neutrophils in the blood are quantified at weeks 8, 12, and 16 following engraftment in the mice engrafted with EMR2KO cells or control cells. The levels of hCD19+ cells, hCD14+ cells, and hCD11b+ cells in the blood are equivalent between the control and EMR2KO groups, and the levels of these cells remained equivalent from weeks 8 to 16 post-engraftment. Comparable levels of hCD19+, hCD14+, and hCD11b+ cells in the blood indicate that similar levels of human myeloid and lymphoid cell populations are present in mice that received the EMR2KO cells and mice that received the control cells.

Finally, amplicon-seq may be performed on bone marrow samples isolated at week 16 post-engraftment to analyze the on-target EMR2 editing in mice that are engrafted with the edited EMR2KO cells.

Evaluating Cell Samples Obtained from the Spleen of Engrafted Animals

At week 16 post-engraftment, the percentages of hCD45+ cells and the percentage of hEMR2+ cells are also quantified in the spleen of mice that are engrafted with control cells or EMR2KO cells. Comparable levels of hCD45+ cells and reduced levels of hEMR2+ cells between the groups of mice (engrafted with control cells or EMR2KO cells) may indicate the long-term persistence of EMR2KO HSCs in the spleens of NSG mice.

Additionally, at week 16 post engraftment, the percentages of hCD14+ monocytes, hCD11b+ granulocytes/neutrophils, CD19+ lymphoid cells, and hCD3+ T cells in the spleen are quantified. Comparable levels of hCD14+ cells, hCD11b+ cells, hCD19+ cells, and hCD3+ in the spleen between the control and EMR2KO groups may indicate that the edited EMR2KO cells are capable of multilineage human hematopoietic cell reconstitution in the spleen of the NSG mice.

### Evaluating Blood and Bone Marrow Neutrophils

At week 16 post engraftment, the percentage of hCD11b+ cells are quantified in the blood and the bone marrow of mice engrafted with control cells or EMR2KO cells. Comparable levels of CD11b+ neutrophil populations observed in the mice engrafted with control cells and the EMR2KO cells in both the blood and the bone marrow of the NSG mice indicates successful engraftment and differentiation.

### Evaluating Blood and Bone Marrow Myeloid and Lymphoid Progenitor Cells

Also, at week 16, the percentage of hCD123+ cells in the blood and the percentage of hCD123+ cells in the bone marrow, and the percentage of hCD10+ cells in the bone marrow are quantified in mice engrafted with control cells or EMR2KO cells. Comparable levels of myeloid and lymphoid progenitor cells between the control and EMR2KO groups may indicate successful engraftment and development.

### Example 7: Editing of EMR2 Using CBE and ABE Base Editors

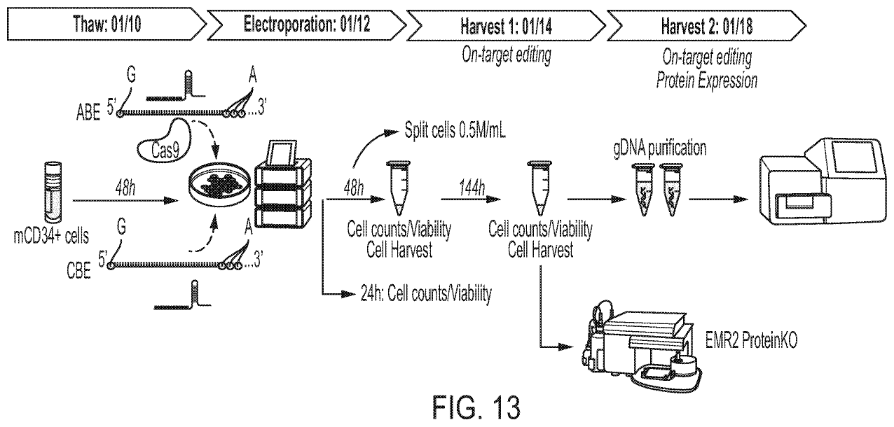

A screening process was employed to evaluate exemplary guide RNAs for editing EMR2 with CBEs and ABEs in CD34+ cells. See, FIG. 13. Examples of gRNAs that were designed and screened for their EMR2 editing efficiency when paired with base editors, such as CBEs and ABEs are found in Tables 3 and 4, as well as Tables 5-7. A control gRNA that does not target EMR2 (gCtrl) having the sequence of GCCGACGCGAAATCTTAGCG (SEQ ID NO: 320) was used in the analyses.

### Viability of Edited Cells

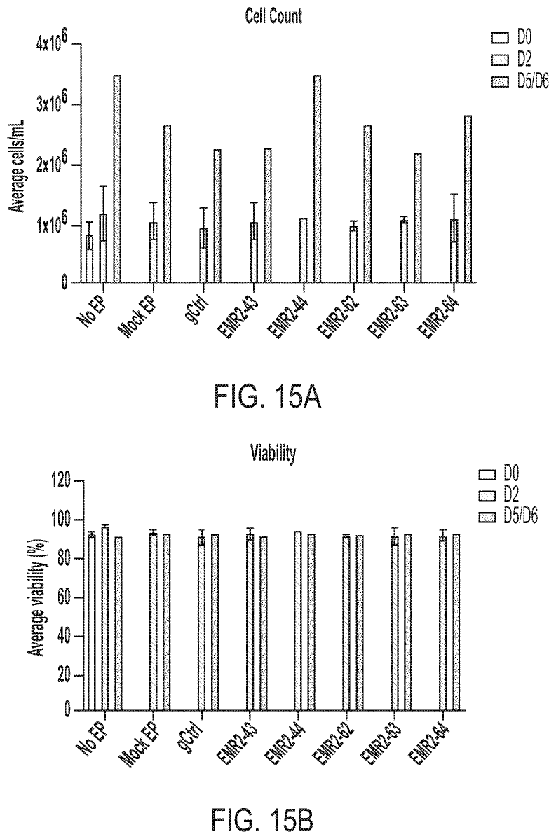

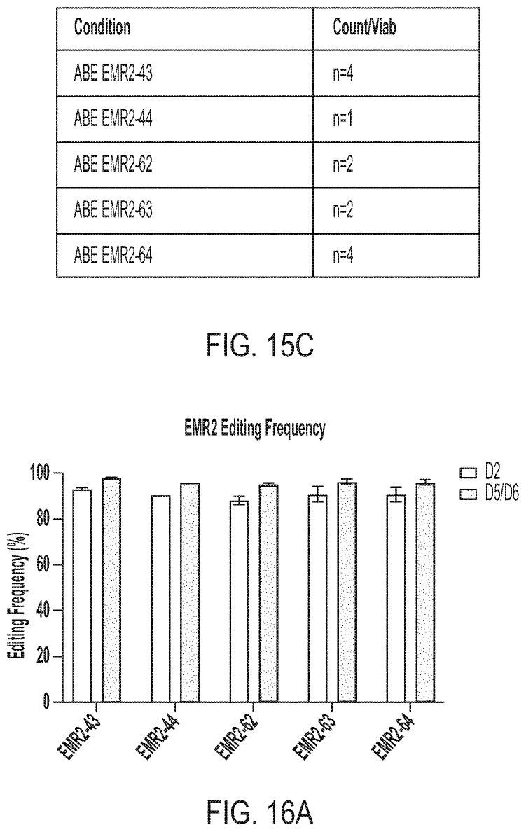

At various time post editing, cell count and cell viability analyses were performed which indicated that editing of EMR2 with CBEs and ABEs caused no reduction in cell viability. See, FIGS. 15A-15C.

### DNA Editing Analyses

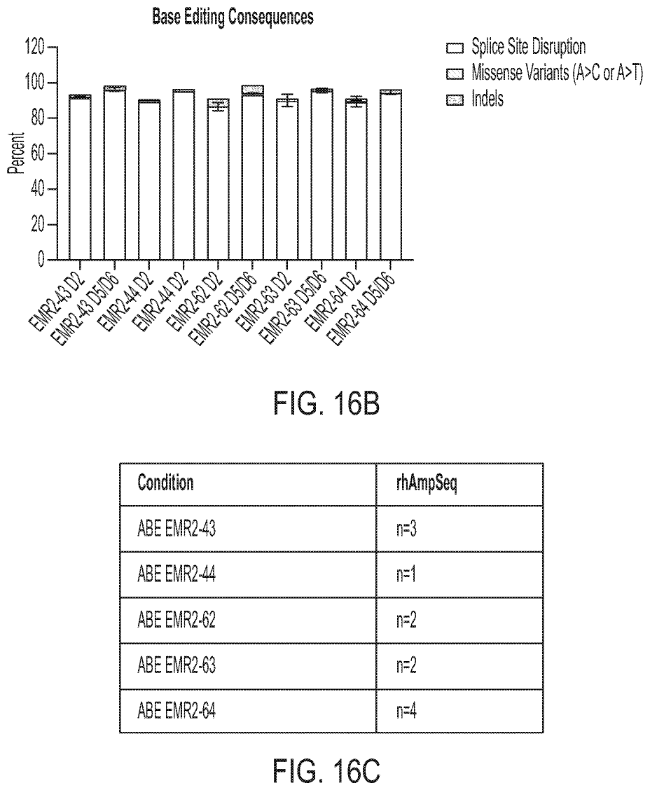

Analysis of EMR2 editing outcomes using CBEs and ABEs in HSCs was performed using rhAmpSeq at various time points post-electroporation. The results indicated high levels of editing and efficient splice site disruption in EMR2. See, FIGS. 16A-16C.

### Flow Cytometry Analysis of EMR2 Protein Expression

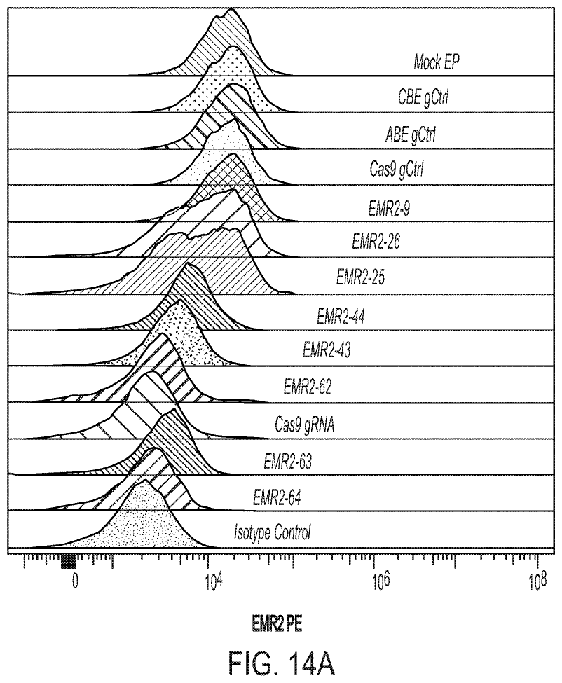

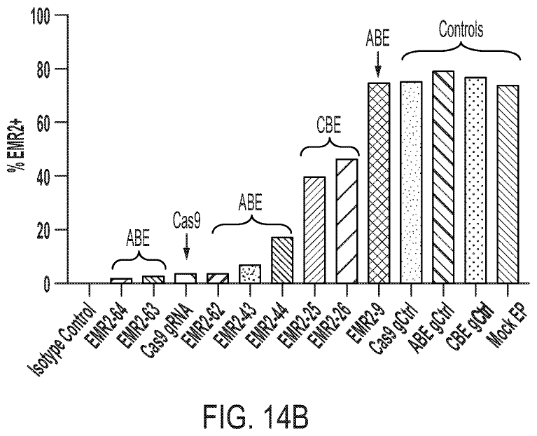

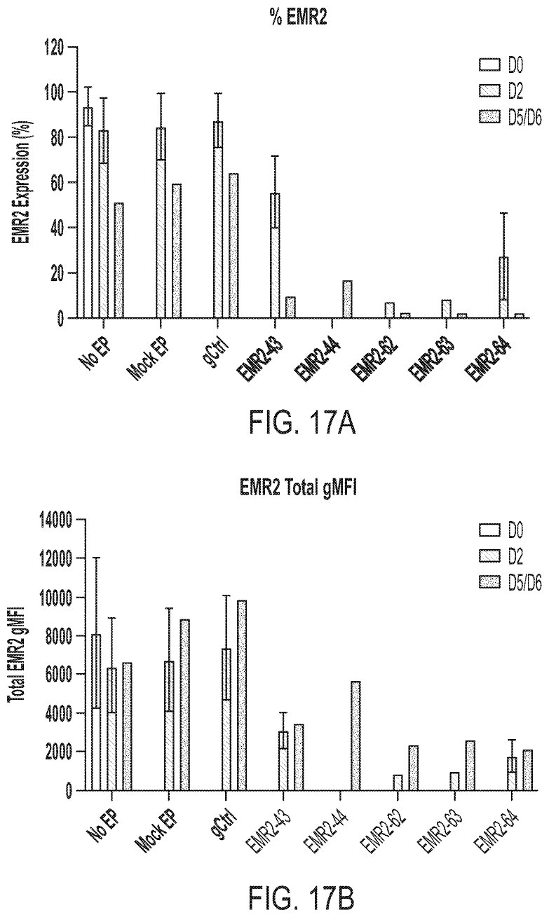

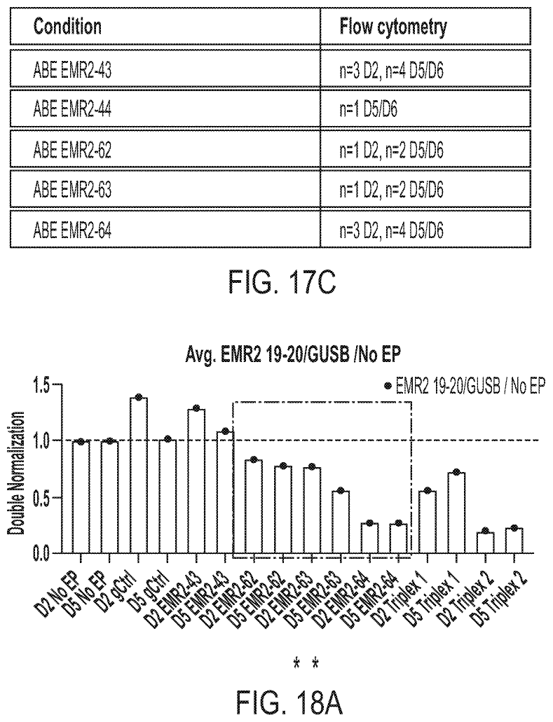

Flow cytometry analyses of EMR2-edited cells electroporated with gRNAs and base editors revealed gRNAs that resulted in significant levels of EMR2 knockdown. See, FIGS. 14A-14B and 17A-17C.

### Transcript Expression Analyses

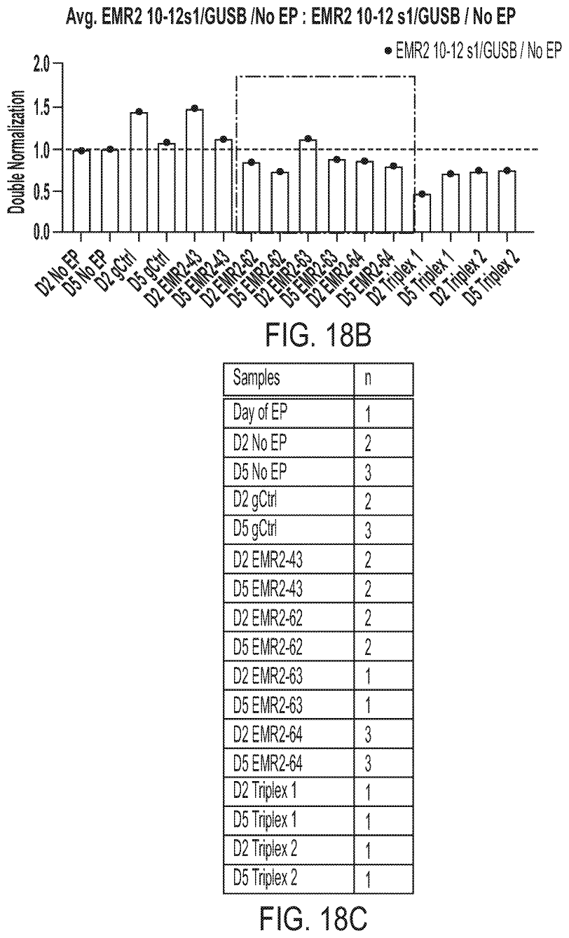

Droplet digital PCR analysis of EMR2 transcript expression indicated 20-50% reductions in EMR2 mRNA transcript levels relative to control cells that underwent no electroporation. See, FIGS. 18A-18C.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A gRNA comprising a targeting domain, wherein the targeting domain comprises a sequence described in Tables 1-8.

2. A gRNA comprising a targeting domain, wherein the targeting domain comprises a sequence of any one of SEQ ID NOs: 59-76, 125-148, 161-166, 179, 180, 183, 184, 186, 189, 192, 195, 201, 204, 207, 210, 213, 216, 219, 222, 225, 228, 231, 234, 237, 249, 243, 246, 249, 252, 254-257, 268, 271, 274, 277, 280, 283, 286, 289, 291, 294, 297, 300, 303, 306, 309, 312, 315, 317, and 219.

3. The gRNA of any one of claims 1 and 2, wherein the gRNA comprises a first complementarity domain, a linking domain, a second complementarity domain which is complementary to the first complementarity domain, and a proximal domain.

4. The gRNA of any one of claims 1-3, wherein the gRNA is a single guide RNA (sgRNA).

5. The gRNA of any one of claims 1-4, wherein the gRNA comprises one or more nucleotide residues that are chemically modified.

6. The gRNA of any one of claims 1-5, wherein the gRNA comprises one or more nucleotide residues that comprise a 2′O-methyl moiety.

7. The gRNA of any one of claims 1-6, wherein the gRNA comprises one or more nucleotide residues that comprise a phosphorothioate.

8. The gRNA of any one of claims 1-7, wherein the gRNA comprises one or more nucleotide residues that comprise a thioPACE moiety.

9. A method of producing a genetically engineered cell, comprising:
a. providing a cell, and
b. contacting the cell with (i) a gRNA of any one of claims 1-8 or a gRNA targeting a targeting domain targeted by a gRNA of any one of claims 1-8; and (ii) an RNA-guided nuclease that binds the gRNA, thus forming a ribonucleoprotein (RNP) complex under conditions suitable for the gRNA of (i) to form and/or maintain an RNP complex with the RNA-guided nuclease of (ii) and for the RNP complex to bind a target domain in the genome of the cell.

10. The method of claim 9, wherein the RNA-guided nuclease is a CRISPR/Cas nuclease.

11. The method of claim 10, wherein the CRISPR/Cas nuclease is a Cas9 nuclease.

12. The method of claim 10, wherein the CRISPR/Cas nuclease is an spCas nuclease.

13. The method of claim 10, wherein the Cas nuclease in an saCas nuclease.

14. The method of claim 10, wherein the CRISPR/Cas nuclease is a Cpf1 nuclease.

15. The method of any one of claims 1-14, wherein the CRISPR/Cas nuclease is a base editor.

16. The method of claim 15, wherein the CRISPR/Cas nuclease is a cytosine base editor or an adenine base editor.

17. The method of any one of claims 9-16, wherein the contacting comprises introducing (i) and (ii) into the cell in the form of a pre-formed ribonucleoprotein (RNP) complex.

18. The method of any one of claims 9-16, wherein the contacting comprises introducing (i) and/or (ii) into the cell in the form of a nucleic acid encoding the gRNA of (i) and/or the RNA-guided nuclease of (ii).

19. The method of any one of claims 9-16 or 18, wherein the nucleic acid encoding the gRNA of (i) and/or the RNA-guided nuclease of (ii) is an RNA, preferably an mRNA or an mRNA analog.

20. The method of any one of claims 9-17, wherein the ribonucleoprotein complex is introduced into the cell via electroporation.

21. The method of any one of claims 9-20, wherein the cell is a hematopoietic cell.

22. The method of any one of claims 9-21, wherein the cell is a hematopoietic stem cell.

23. The method of any one of claims 9-22, wherein the cell is a hematopoietic progenitor cell.

24. The method of any one of claims 9-20, wherein the cell is an immune effector cell.

25. The method of any one of claims 9-20 or 24, wherein the cell is a lymphocyte.

26. The method of any one of claims 9-20, 24, or 25, wherein the cell is a T-lymphocyte.

27. A genetically engineered cell, wherein the cell is obtained by the method of any one of claims 9-26.

28. A cell population, comprising the genetically engineered cell of claim 27.

29. A cell population, comprising a genetically engineered cell, wherein the genetically engineered cell comprises a genomic modification that consists of an insertion or deletion immediately proximal to a site cut by an RNA-guided nuclease when bound to a gRNA comprising a targeting domain as described in any of Tables 1-8.

30. The cell population of claim 29, wherein the genomic modification is an insertion or deletion generated by a non-homologous end joining (NHEJ) event.

31. The cell population of claim 29, wherein the genomic modification is an insertion or deletion generated by a homology-directed repair (HDR) event.

32. The cell population of any one of claims 29-31, wherein the genomic modification results in a loss-of function of EMR2 in a genetically engineered cell harboring such a genomic modification.

33. The cell population of any one of claims 29-32, wherein the genomic modification results in a reduction of expression of EMR2 to less than 25%, less than 20% less than 10% less than 5% less than 2% less than 1%, less than 0.1%, less than 0.01%, or less than 0.001% as compared to the expression level of EMR2 in wild-type cells of the same cell type that do not harbor a genomic modification of EMR2.

34. The cell population of any one of claims 29-33, wherein the genetically engineered cell is a hematopoietic stem or progenitor cell.

35. The cell population of any one of claims 29-33, wherein the genetically engineered cell is an immune effector cell.

36. The cell population of any one of claims 29-33 or 35, wherein the genetically engineered cell is a T-lymphocyte.

37. The cell population of any one of claims 35 and 36, wherein the immune effector cell expresses a chimeric antigen receptor (CAR).

38. The cell population of claim 37, wherein the CAR targets EMR2.

39. The cell population of any one of claims 28-34, which is characterized by the ability to engraft EMR2-edited hematopoietic stem cells in the bone marrow of a recipient and to generate differentiated progeny of all blood lineage cell types in the recipient.

40. The cell population of any one of claims 28-34 or 39, which is characterized by the ability to engraft EMR2-edited hematopoietic stem cells in the bone marrow of a recipient at an efficiency of at least 50%.

41. The cell population of any one of claims 28-34, 39, or 40, which is characterized by the ability to engraft EMR2-edited hematopoietic stem cells in the bone marrow of a recipient at an efficiency of at least 60%.

42. The cell population of any one of claims 28-34 or 39-41, which is characterized by the ability to engraft EMR2-edited hematopoietic stem cells in the bone marrow of a recipient at an efficiency of at least 70%.

43. The cell population of any one of claims 28-34 or 39-42, which is characterized by the ability to engraft EMR2-edited hematopoietic stem cells in the bone marrow of a recipient at an efficiency of at least 80%.

44. The cell population of any one of claims 28-34 or 39-43, which is characterized by the ability to engraft EMR2-edited hematopoietic stem cells in the bone marrow of a recipient at an efficiency of at least 90%.

45. The cell population of any one of claims 28-34 or 39-44, wherein the cell population comprises EMR2-edited hematopoietic stem cells that are characterized by a differentiation potential that is equivalent to the differentiation potential of non-edited hematopoietic stem cells.

46. A method, comprising administering to a subject in need thereof the genetically engineered cell of claim 27 or the cell population of any one of claims 28-45.

47. The method of claim 46, wherein the subject has or has been diagnosed with a hematopoietic malignancy.

48. The method of claim 46 or 47, further comprising administering to the subject an effective amount of an agent that targets EMR2, wherein the agent comprises an antigen-binding fragment that binds EMR2.
